(19) United States
(12) Patent Application Publication
(10) Pub. No.: US 2025/0368717 A1
(43) Pub. Date: Dec. 4, 2025

(54) COMMON TUMOR-SPECIFIC T CELL RECEPTORS

(71) Applicant: HS DIAGNOMICS GMBH, Berlin (DE)

(72) Inventors: Steffen HENNIG, Berlin (DE); Rudolf HAMMER, Zornheim (DE)

(73) Assignee: HS DIAGNOMICS GMBH, Berlin (DE)

(21) Appl. No.: 18/870,674

(22) PCT Filed: May 30, 2023
(86) PCT No.: PCT/EP2023/064399

§ 371 Date: Dec. 1, 2024

(30) Foreign Application Priority Data
May 30, 2022 (EP) 22176133.1
Jan. 18, 2023 (EP) 23152250.9

(51) Int. Cl. C07K 14/725; A61K 40/11; A61K 40/32; A61P 35/00; C12N 5/0783

(52) U.S. Cl. CPC C07K 14/7051; A61K 40/11; A61K 40/32; A61P 35/00; C12N 5/0636; C12N 2510/00

(57) ABSTRACT

The invention relates to common patient-spanning tumor-specific T cell receptors (TCRs), a nucleic acid encoding the TCR, and a T cell comprising the TCR and/or the encoding nucleic acid, and to these agents for use in cancer therapy.

This application claims the right of priority of European Patent Application EP22176133.1 filed 30 May 2022, and of European Patent Application EP23152250.9 filed 18 Jan. 2023, both incorporated by reference herein.

### FIELD OF THE INVENTION

The invention relates to common patient-spanning tumor-specific T cell receptors (TCRs), a nucleic acid encoding the TCR, and a T cell comprising the TCR and/or the encoding nucleic acid, and to these agents for use in cancer therapy.

### BACKGROUND OF THE INVENTION

Ever since it has been shown that the immune system is capable of combatting and rejecting tumors, great efforts have been made to develop therapeutic or preventative cancer vaccines. These efforts were met with formidable challenges in antigen discovery since suitable tumor antigens for vaccination need to combine three basic requirements. They have to be immunogenic to elicit an efficacious therapeutic response. They need to be tumor-specific to allow a safe treatment, especially in the preventative setting. However, a cross-reactivity with pathogen-associated antigens is within the scope of the invention. Finally, there is the need to identify shared antigens which are expressed in tumors of many patients.

In the last decade two high throughput platforms were developed and extensively used in tumor antigen discovery. Both, however, fulfill the requirements only to a limited extent. Whole exome sequencing approaches clearly allow the identification of tumor-specific mutated antigens. But except certain recurrent driver mutations the vast majority of identified mutation reactive antigens are patient specific. On the other hand, mass spectroscopy-based approaches are able to detect shared HLA-presented peptides in tumors of different patients. But proof of immunogenicity and tumor-specificity for those peptides remain challenging tasks.

Adoptive cell therapy (ACT) with T cells genetically engineered to express tumor-reactive chimeric antigen receptors (CAR-T cells) or T-cell receptors (TCRs) is a promising treatment strategy for patients with cancer. In contrast to hematologic malignancies where CAR-T cells against certain lineage-specific cell surface antigens have been approved because of their high efficacy with manageable side effects, for solid cancers, the application of CAR-T cells is (currently) not feasible due to the lack of cell surface target antigens with tumor-restricted expression. (Patient) T cells genetically engineered to express tumor-specific transgenic TCRs (tsTCRtg-T cells) recognizing peptides from tumor-associated or tumor-specific antigens (TAA or TSA) presented by HLA-molecules (pMHC) represent attractive alternatives. While TAA (e.g. Cancer/Germline-, differentiation-, overexpressed antigens) and viral (v) TSA (in tumors with viral etiology) can be widely shared between tumors and result in the presentation of common pMHC in HLA-matched patients, the vast majority of (non-viral) TSA (neoantigens) are unique to individual cancers. The resulting vast variety of pMHC have to be regarded as private antigens of individual subjects. However, in a small number of cases, TSA resulting from point mutations or chromosomal translocations that affect common driver genes of malignancy and are shared between tumors have been shown to be immunogenic (e.g. RAS-, TP53-, BRAF-, PIK3CA-mutations and translocations involving ALK, ROS, NTRK, RET, etc.). Also, thus far less well-defined antigen categories like tumor-specific cryptic (“dark matter”) or aberrantly spliced transcripts, have the potential to be shared between tumors and recognized by T cells.

Previously, the inventors have developed a method that identifies tumor-specific T-cell receptors by comparing CDR3 sequences obtained from TILs with T-cells in the adjacent tissue (WO 2017/025564 A1). Thus, it is possible to delineate tumor-specificity by the increased presence of T-cell clones in the tumor vs. non-tumor of a patient. However, most tumor-specific antigens arise through mutations that are limited to the individual patient. Whereas private neoantigens can be targeted by personalized tsTCRtg-T cell therapies, shared TAA or TSA are ideal targets for off-the-shelf tsTCRtg-T cell therapies in patients with expression of matched HLA alleles. Personalized therapy is time-consuming, costly and highly regulated by FDA and EMA (ATMP, advanced medicinal products; gene therapy medicinal products). In addition, many patients' diseases progress faster than personalized therapeutics can be produced. Therefore, it would be highly advantageous to develop a method that identifies carriers of such common tumor-specific TCRs by scanning their TIL-repertoires for identical or highly similar antigen-recognition domains (CDR3α and -β) thereby providing off-the-shelf therapeutic receptors and concomitantly opening up an opportunity to identify the shared tumor-specific antigens for additional therapeutic options.

Based on the above-mentioned state of the art, the objective of the present invention is to provide common tumor-specific TCR sequences. This objective is attained by the subject-matter of the independent claims of the present specification, with further advantageous embodiments described in the dependent claims, examples, figures and general description of this specification.

### SUMMARY OF THE INVENTION

Rather than starting with antigen candidates that need critical validations in specificity and functional tests, an alternative way is to analyze T cell repertoires in tumors of different cancer patients searching for specific effects originating from shared tumor antigens. When a T-cell infiltrates the tumor and provokes a specific receptor mediated interaction with a tumor antigen, this encounter is followed by activation, proliferation and enrichment of the clone in the tumor. Thus, the preferred localization of this unique TCR clonotype, as determined quantitatively by the ratio of the TCR clonotype frequencies between tumor and adjacent non-tumor tissue is a predictor of tumor-specificity. This technology is described in WO 2017/025564 A1. If such a unique tumor-specific TCR clonotype or structurally closely related TCR clonotypes, referred to as a TCR cluster, are detected in the tumors of other patients, this is indicative of the existence of a shared tumor antigen in these patients. This is particularly informative when TCR clusters are detected in HLA-matched patients revealing the nature of the HLA allele presenting the shared antigenic epitope. As the last step, complete elucidation of the cluster TCRs, e.g. by single cell technologies, will yield α/β-TCRs with specificity for the shared antigen.

Such HLA restricted α/β-TCRs with specificity for shared tumor antigens are the starting point of important applications.

As unprecedented novel tools they can be used as specific probes in antigen discovery guiding the targeted search for shared tumor antigens.

As “off the shelf” TCRs in vector form they can be used for transduction into autologous T cells of cancer patients for immunotherapeutic intervention. Eligible are HLA-matched patients who are either carriers of cluster TCRs or are carriers of the known shared tumor antigen.

Owing to novel genetic engineering technologies (CRISPR/Cas9, TALEN, zinc finger nucleases), it more and more becomes feasible to produce allogeneic cellular therapeutic products from healthy donors which are more readily available and at higher numbers than from most patients, and a single product can be used for the treatment of several patients. This is possible because (autologous as well as allogeneic) tsTCRtg-T cells can be genetically engineered to be less immunogenic (e.g. via knock-out of endogenous HLAs in the allogeneic setting), less prone to exhaustion/dysfunction (e.g. per knockout of checkpoint receptors), and less susceptible to induce Graft-versus-host disease (GvHD) or unpredictable crossreactivity due to the knock-out of the endogenous TCRs.

In addition to transducing conventional autologous or allogeneic CD4+ and CD8+ T cells with α/β-tsTCR, it is an option to transduce additional types of adaptive or innate immune cells, like γδ-T cells, NKT cells, and NK cells with the receptors; the genetic engineering technologies mentioned above enable co-transduction of NK cells with tsTCRs and the CD3-signalling domains necessary for the activation of the cells upon TCR-engagement with pMHC.

Therefore, it is highly advantageous to find shared tumor antigens and/or T-cell receptors that are common to more than one individual.

Hence, there is a need to identify shared tumor-specific antigens and/or shared tumor-specific T-cell receptors. This would enable an off-the-shelf treatment for cancer given that the HLAs of the patient are known to match.

A first aspect of the invention relates to an isolated TCR characterized by certain CDR3 sequences.

A second aspect of the invention relates to a nucleic acid sequence encoding the TCR according to the first aspect.

A third aspect of the invention relates to an isolated autologous T cell comprising a TCR according to the first aspect, and/or a nucleic acid sequence according to the second aspect.

A fourth aspect of the invention relates to the TCR according to the first aspect, the nucleic acid sequence according to the second aspect, or the isolated autologous T cell according to the third aspect for use in treatment of cancer.

In another embodiment, the present invention relates a pharmaceutical composition comprising at least one of TCR, nucleic acid sequence, or isolated autologous T cell of the present invention and at least one pharmaceutically acceptable carrier, diluent or excipient.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “1X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

### Sequences

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

### General Biochemistry: Peptides, Amino Acid Sequences

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

### General Molecular Biology: Nucleic Acid Sequences, Expression

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of a RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The term Nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). Wherever reference is made herein to a hybridizing sequence, such hybridizing sequence may be composed of any of the above nucleotides, or mixtures thereof.

### T Cell Biology

The term “CDR3” in the context of the present specification refers to the hypervariable complementarity determining region 3. The size of CDR3 is particularly characterized by the total number of amino acids (AA) and respective nucleotides from the conserved cysteine in the Vβ, or Vα or Vγ or Vδ segment to the position of the conserved phenylalanine in the Jβ or Jα, Jγ or Jδ segment.

The term “TCR” or “TCR polypeptide” in the context of the present specification refers to a T cell receptor. Depending on the context, the term TCR encompasses either
1) a heterodimeric transmembrane protein composed of one alpha and one beta chain expressed in T cells in a native configuration and associated with accessory proteins for signal transduction;
2) a soluble truncated derivate of 1) composed of the variable domains of one alpha and one beta chain in their native (antigen binding) configuration and expressed as fusion construct with a variety of fusion partners providing a variety of effector functions.

The minimal requirement for a TCR is that it comprises (at least a truncated version of) an alpha and a beta chain which comprise the CDR3 regions and are able to bind an antigen specifically.

### (Cancer) Immunotherapy

The term “HLA” in the context of the present invention refers to the human leukocyte antigen, as a specific subset of the general term major histocompatibility complex (MHC).

HLA supertypes have been defined based on grouping together MHC alleles that share similar binding specificities, i.e. peptides with same or similar so-called anchor amino acid residues (e.g. positions 2 and 9 or 10 in 9- and 10mer peptides). HLA supertypes are further described in Sidney et al. (BMC Immunology 2008, 9:1).

The term essentially identical in the context of the present specification relates to nucleic acid sequences which are either identical or have an identity of at least 95%, particularly of at least 97%, more particularly of at least 98%, more particularly of at least 99%, most particularly of more than 99

The term gene of the same HLA-type in the context of the present specification relates to the HLA-genes encoding MHC molecules. The same HLA-type herein means that the HLA gene encodes the same variant of an MHC molecule. As there is a large variety of HLA genes in mankind, the HLA repertoire of the tested patients is determined in one embodiment of the method of the invention, and patients sharing at least one gene of the same HLA-type are selected for further analysis.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

The term mutation of a gene or a protein refers to an alteration of the nucleic acid sequence or the amino acid sequence. This alteration leads to a difference in the activity of the respective protein. Difference of activity means that the signal pathway—in which the protein is involved—is upregulated in case of KRAS, EGFR, FGFR, or BRAF and downregulated in case of TP53.

The term KRAS refers to a gene of GeneiD 3845 or a protein of UniProt-ID P01116.

The term EGFR refers to a gene of GeneiD 1956 or a protein of UniProt-ID P00533.

The term FGFR1 refers to a gene of GeneiD 2260 or a protein of UniProt-ID P11362.

The term BRAF refers to a gene of GeneiD 673 or a protein of UniProt-ID P15056.

The term TP53 refers to a gene of GeneiD 7157 or a protein of UniProt-ID P04637.

The term KRAS G12 mutation refers to a substitution of glycine at position 12 of the KRAS protein for a different amino acid.

The term KRAS Q61 mutation refers to a substitution of glutamine at position 61 of the KRAS protein for a different amino acid.

The term mutation in a gene of the EGFR-Raf-Ras pathway refers to a mutation of a gene in this pathway. The gene may be selected from a growth factor receptor, KRAS, BRAF, MEK, and ERK.

### DETAILED DESCRIPTION OF THE INVENTION

TCR-Clusters Correlate with Presence of Tumor Driver Mutations

One central aspect of this invention is the significant occurrence of known tumor driver mutations, mainly from the KRAS-family, in tumors of patients whose TCRs are found in large clusters of very similar TCRs where ‘similar’ refers to peptide sequences of ideally both chains (alpha and beta) of the TCRs which together build the functional TCR. The TCR clusters are comprising different patients which in most cases share HLA-types. In addition, the inventors provide evidence that many of the clusters are comprising patients with lung and pancreatic cancers (see clusters a, e, j, l,m,p).

The finding of common mutations (table 17) does not mean that the respective mutations are direct sources of tumor antigens, i.e. that peptides being part of the mutated protein are presented as neoantigens by tumor cells via their MHC molecules. The cognate and functional antigens may be products of oncogenic signal transduction pathway activation resulting in the expression of gene transcripts under control and/or modified by activity of the respective driver mutation. This has far reaching consequences for the application of cluster TCRs in therapy and diagnostics:
a. Validation of the cluster TCRs' direct association with oncogenic pathway activation involving de-novo generation of (unknown) tumor-specific antigens renders the TCRs selectable as tumor-specific TCRs. The TCR selection is antigen-agnostic and suggests safe application of the TCRs in adoptive cellular therapy (ACT) approaches with TCR-transduced T cells (TCR-T cells).
b. In newly diagnosed patients, the existence of cluster TCRs found either in tumor tissue (e.g. via needle biopsies or archived tumor material) or peripheral blood of the patients sharing the relevant HLA-type identifies them as prospective recipients of a TCR-T cell therapy* (by administration of a high number of T-cells equipped with validated cluster TCRs). *TCR-T cell therapy (short: TCR-T therapy): Cellular therapy using autologous/allogeneic T-cells equipped with disease-specific T-cell receptors (TCR)
c. The identification of a panel of tumor mutations is a standard diagnostic tool in current oncological practice, mainly performed by deep sequencing technology. The identification of mutations in a patient's tumor, which are associated with cluster TCRs, is a strong indicator that the patient will benefit from a TCR-T therapy with cluster TCR-transduced T cells, provided that the patient exhibits the cluster-associated HLA-type. This may even be true if the patient has no measurable frequencies of respective cluster TCRs.
d. For tumor patients fulfilling both criteria, b and c, there is strong evidence that they will benefit from a TCR-T therapy even if the respective cancer type is not yet included in the clusters a-o.
   TCR-T Therapy with Cluster TCRs

The identification of cluster TCRs in different patients of one cancer type or even different cancer types is a very promising basis for TCR-T therapies. The respective T-cells for therapeutic use can be produced either by
a. Transduction of autologous/allogeneic T cells with recombinant TCR-constructs derived from peripheral blood and ex vivo-expansion or
b. expansion of selected endogenous T-cells expressing cluster-TCRs isolated from the patients' tumor-infiltrating lymphocytes or peripheral blood lymphocytes. Isolation of these pre-existing autologous cluster T-cells can be achieved by enrichment and FACS using one or more specific anti-TCR-ligands (e.g. antibodies) or multimeric HLA/peptide complexes before the expansion step.

In any case, patients can be stratified before TCR-T therapy by screening blood or even tumor samples with DNA sequencing technologies and methods developed by the inventors to identify TCR sequences from blood or tissue samples. Once patients are carrying TCRs identical or very similar to known cluster TCRs and are of the respective HLA-type they can be expected to benefit from a corresponding TCR-T therapy.

A first aspect of the invention relates to an isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein
a. for group a, the CDR3 alpha sequence is selected from the group of sequences comprising SEQ ID NO 007-009 or SEQ ID NO 436-439, and the CDR3 beta sequence is selected from the group of sequences comprising SEQ ID NO 001-006 or SEQ ID NO 432-435, or
b. for group b, the CDR3 alpha sequence is selected from the group of sequences comprising SEQ ID NO 031-032, and the CDR3 beta sequence is selected from the group of sequences comprising SEQ ID NO 027-030, or
c. for group c, the CDR3 alpha sequence is selected from the group of sequences comprising SEQ ID NO 052-054, and the CDR3 beta sequence is selected from the group of sequences comprising SEQ ID NO 048-051, or
d. for group d, the CDR3 alpha sequence is selected from the sequence SEQ ID NO 073, and the CDR3 beta sequence is selected from the group of sequences comprising SEQ ID NO 070-072, or
e. for group e, the CDR3 alpha sequence is selected from the sequence SEQ ID NO 100 or SEQ ID NO 455-459 and the CDR3 beta sequence is selected from the group of sequences comprising SEQ ID NO 086-099 or SEQ ID NO 450-454, or
f. for group f, the CDR3 alpha sequence is selected from the group of sequences comprising SEQ ID NO 131-132 or SEQ ID NO 472-473, and the CDR3 beta sequence is selected from the group of sequences comprising SEQ ID NO 124-130 or SEQ ID NO 470-471, or
g. for group g, the CDR3 alpha sequence is selected from the sequence SEQ ID NO 156, and the CDR3 beta sequence is selected from the group of sequences comprising SEQ ID NO 150-155, or
h. for group h, the CDR3 alpha sequence is selected from the group of sequences comprising SEQ ID NO 175-176, and the CDR3 beta sequence is selected from the group of sequences comprising SEQ ID NO 172-174, or
i. for group i, the CDR3 alpha sequence is selected from the group of sequences comprising SEQ ID NO 193-194, and the CDR3 beta sequence is selected from the group of sequences comprising SEQ ID NO 190-192, or
j. for group j, the CDR3 alpha sequence is selected from the group of sequences comprising SEQ ID NO 212-213 or SEQ ID NO 513, and the CDR3 beta sequence is selected from the group of sequences comprising SEQ ID NO 208-211 or SEQ ID NO 478, or
k. for group k, the CDR3 alpha sequence is selected from the group of sequences comprising SEQ ID NO 232-235, and the CDR3 beta sequence is selected from the group of sequences comprising SEQ ID NO 228-231, or
l. for group l, the CDR3 alpha sequence is selected from the group of sequences comprising SEQ ID NO 261-269 or SEQ ID NO 484-486, and the CDR3 beta sequence is selected from the group of sequences comprising SEQ ID NO 252-260 or SEQ ID NO 481-483, or
m. for group m, the CDR3 alpha sequence is selected from the group of sequences comprising SEQ ID NO 302-307 or SEQ ID NO 496-498, and the CDR3 beta sequence is selected from the group of sequences comprising SEQ ID NO 296-301 or SEQ ID NO 493-495, or
n. for group n, the CDR3 alpha sequence is selected from the group of sequences comprising SEQ ID NO 334-339, and the CDR3 beta sequence is selected from the group of sequences comprising SEQ ID NO 328-333, or
o. for group o, the CDR3 alpha sequence is selected from the group of sequences comprising SEQ ID NO 363-365, and the CDR3 beta sequence is selected from the group of sequences comprising SEQ ID NO 360-362, or
p. for group p, the CDR3 alpha sequence is selected from the group of sequences comprising SEQ ID NO 391-401 or SEQ ID NO 507-508, and the CDR3 beta sequence is selected from the group of sequences comprising SEQ ID NO 380-390 or SEQ ID NO 505-506,
   particularly wherein the CRD3 alpha sequence and the CDR3 beta sequence are identified in the same row of tables 1-16,

In certain embodiments, the substitutions are selected according to the substitution rules given below,
wherein the substitution rules are:
   glycine (G) and alanine (A) are interchangeable; valine (V), leucine (L), and isoleucine (I) are interchangeable, A and V are interchangeable;
   tryptophan (W) and phenylalanine (F) are interchangeable, tyrosine (Y) and F are interchangeable;
   serine (S) and threonine (T) are interchangeable;
   aspartic acid (D) and glutamic acid (E) are interchangeable
   asparagine (N) and glutamine (Q) are interchangeable; N and S are interchangeable; N and D are interchangeable; E and Q are interchangeable;
   methionine (M) and Q are interchangeable;
   cysteine (C), A and S are interchangeable;
   proline (P), G and A are interchangeable;
   arginine (R) and lysine (K) are interchangeable.

A group of CDR3 sequences may also be called a cluster.

In certain embodiments, the CDR3 sequences are selected from the groups a, b, c, d, e, f, g, h, i, j, k.

In certain embodiments, the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein the complete TCR sequence retains its biological activity, wherein
a. for group a, the V alpha sequence is SEQ ID NO 025, the JC alpha sequence is SEQ ID NO 026, the V beta sequence is SEQ ID NO 018, and the JC beta sequence is SEQ ID NO 019, or
b. for group b, the V alpha sequence is SEQ ID NO 046, the JC alpha sequence is SEQ ID NO 047, the V beta sequence is SEQ ID NO 040, and the JC beta sequence is SEQ ID NO 041, or
c. for group c, the V alpha sequence is SEQ ID NO 068, the JC alpha sequence is SEQ ID NO 069, the V beta sequence is SEQ ID NO 061, and the JC beta sequence is SEQ ID NO 062, or
d. for group d, the V alpha sequence is SEQ ID NO 084, the JC alpha sequence is SEQ ID NO 085, the V beta sequence is SEQ ID NO 079, and the JC beta sequence is SEQ ID NO 080, or
e. for group e, the V alpha sequence is SEQ ID NO 122, the JC alpha sequence is SEQ ID NO 123, the V beta sequence is SEQ ID NO 117, and the JC beta sequence is SEQ ID NO 118, or
f. for group f, the V alpha sequence is SEQ ID NO 148, the JC alpha sequence is SEQ ID NO 149, the V beta sequence is SEQ ID NO 142, and the JC beta sequence is SEQ ID NO 143, or
g. for group g, the V alpha sequence is SEQ ID NO 170, the JC alpha sequence is SEQ ID NO 171, the V beta sequence is SEQ ID NO 165, and the JC beta sequence is SEQ ID NO 166, or
h. for group h, the V alpha sequence is SEQ ID NO 188, the JC alpha sequence is SEQ ID NO 189, the V beta sequence is SEQ ID NO 182, and the JC beta sequence is SEQ ID NO 183, or
i. for group i, the V alpha sequence is SEQ ID NO 206, the JC alpha sequence is SEQ ID NO 207, the V beta sequence is SEQ ID NO 200, and the JC beta sequence is SEQ ID NO 201, or
j. for group j, the V alpha sequence is SEQ ID NO 226, the JC alpha sequence is SEQ ID NO 227, the V beta sequence is SEQ ID NO 220, and the JC beta sequence is SEQ ID NO 221, or
k. for group k, the V alpha sequence is SEQ ID NO 250, the JC alpha sequence is SEQ ID NO 251, the V beta sequence is SEQ ID NO 242, and the JC beta sequence is SEQ ID NO 243, or
l. for group l, the V alpha sequence is SEQ ID NO 294, the JC alpha sequence is SEQ ID NO 295, the V beta sequence is SEQ ID NO 281, and the JC beta sequence is SEQ ID NO 282, or
m. for group m, the V alpha sequence is SEQ ID NO 326, the JC alpha sequence is SEQ ID NO 327, the V beta sequence is SEQ ID NO 316, and the JC beta sequence is SEQ ID NO 317, or
n. for group n, the V alpha sequence is SEQ ID NO 358, the JC alpha sequence is SEQ ID NO 359, the V beta sequence is SEQ ID NO 348, and the JC beta sequence is SEQ ID NO 349, or
o. for group o, the V alpha sequence is SEQ ID NO 378, the JC alpha sequence is SEQ ID NO 379, the V beta sequence is SEQ ID NO 371, and the JC beta sequence is SEQ ID NO 372, or
p. for group p, the V alpha sequence is SEQ ID NO 430, the JC alpha sequence is SEQ ID NO 431, the V beta sequence is SEQ ID NO 415, and the JC beta sequence is SEQ ID NO 416.

The biological activity of a TCR is determined via the activation of the T cell comprising the TCR by a tumor cell or an APC. If the TCR is still able to recognize an APC presenting the HLA-peptide complex for which the TCR is specific, then the TCR retains its biological activity, even in cases with a deviating sequence.

A second aspect of the invention relates to a nucleic acid sequence encoding the TCR polypeptide according to the first aspect.

A third aspect of the invention relates to an isolated autologous T cell comprising a TCR polypeptide according to the first aspect. An alternative of the third aspect of the invention relates to an isolated autologous T cell comprising a nucleic acid sequence according to the second aspect.

In certain embodiments, the isolated autologous T cell is a recombinant T cell recombinantly expressing said TCR polypeptide.

A fourth aspect of the invention relates to the TCR polypeptide according to the first aspect for use in treatment of cancer. An alternative of the fourth aspect of the invention relates to the nucleic acid sequence according to the second aspect for use in treatment of cancer. An alternative of the fourth aspect of the invention relates to the isolated autologous T cell according to the third aspect for use in treatment of cancer.

In certain embodiments of the fourth aspect, the agent of the fourth aspect is administered to a patient characterized by the following HLA-type:
a. HLA-A*02:01 for group a; or
b. HLA-B*08:01 and/or HLA-C*07:01 for group b; or
c. HLA-A*02:01 for group c; or
d. HLA-A*02:01 for group d; or
e. HLA-B*15:01 for group e; or
f. HLA-A*02:01 for group f; or
g. HLA-B*08:01 for group g; or
h. HLA-B*07:02 for group h; or
i. HLA-A*01:01 and/or HLA-B*08:01 and/or HLA-C*07:01 for group i; or
j. HLA-A*02:01 for group j; or
k. HLA-A*02:01 for group k.

In certain embodiments of the fourth aspect, the cancer is a solid tumor. In certain embodiments of the fourth aspect, the cancer is selected from lung cancer, pancreatic cancer, colon cancer, and breast cancer. In certain embodiments of the fourth aspect, the cancer is selected from lung cancer and pancreatic cancer.

In certain embodiments of the fourth aspect, the cancer is selected from the group of Bladder Urothelial Carcinoma, Breast invasive carcinoma, Cervical squamous cell carcinoma and endocervical adenocarcinoma, Cholangiocarcinoma, Colon adenocarcinoma, Lymphoid Neoplasm Diffuse Large B-cell Lymphoma, Esophageal carcinoma, Glioblastoma multiforme, Head and Neck squamous cell carcinoma, Kidney Chromophobe, Kidney renal papillary cell carcinoma, Acute Myeloid Leukemia, Brain Lower Grade Glioma, Lung adenocarcinoma, Lung squamous cell carcinoma, Mesothelioma, Ovarian serous cystadenocarcinoma, Pancreatic adenocarcinoma, Rectum adenocarcinoma, Sarcoma, Skin Cutaneous Melanoma, Stomach adenocarcinoma, Testicular Germ Cell Tumors, Thyroid carcinoma, Uterine Corpus Endometrial Carcinoma, Uterine Carcinosarcoma.

In certain embodiments of the fourth aspect, the cancer is characterized by a mutation in a gene selected from the group of KRAS, EGFR, and/or TP53 for group a, particularly wherein the mutation of KRAS is a KRAS G12 mutation.

In certain embodiments of the fourth aspect, the cancer is characterized by a mutation in a gene selected from the group of KRAS, FGFR, and/or TP53 for group b, particularly wherein the mutation of KRAS is a KRAS Q61 mutation.

In certain embodiments of the fourth aspect, the cancer is characterized by a mutation in a gene selected from the group of KRAS, and/or EGFR for group c, particularly wherein the mutation of KRAS is a KRAS G12 or a KRAS Q61 mutation.

In certain embodiments of the fourth aspect, the cancer is characterized by a mutation in a gene selected from the group of KRAS and/or TP53 for group d, particularly wherein the mutation of KRAS is a KRAS G12 mutation.

In certain embodiments of the fourth aspect, the cancer is characterized by a mutation in a gene selected from the group of KRAS, EGFR and/or BRAF for group e, particularly wherein the mutation of KRAS is a KRAS G12 or a KRAS Q61 mutation.

In certain embodiments of the fourth aspect, the cancer is characterized by a mutation in a gene selected from the group of KRAS, EGFR and/or BRAF for group f, particularly wherein the mutation of KRAS is a KRAS G12 or a KRAS Q61 mutation.

In certain embodiments of the fourth aspect, the cancer is characterized by a mutation in a gene TP53 for group g.

In certain embodiments of the fourth aspect, the cancer is characterized by a mutation in a gene selected from the group of KRAS, EGFR, BRAF and/or TP53 for group h, particularly wherein the mutation of KRAS is a KRAS G12 or a KRAS Q61 mutation.

In certain embodiments of the fourth aspect, the cancer is characterized by a mutation in a gene selected from the group of EGFR, FGFR, and/or TP53 for group i.

In certain embodiments of the fourth aspect, the cancer is characterized by a mutation in a gene selected from the group of KRAS, EGFR and/or TP53, particularly wherein the mutation of KRAS is a KRAS G12 mutation.

In certain embodiments of the fourth aspect, the cancer is characterized by a mutation in a gene selected from the group of KRAS, EGFR, BRAF and/or TP53 for group k, particularly wherein the mutation of KRAS is a KRAS G12 or a KRAS Q61 mutation.

In certain embodiments of the fourth aspect, the cancer is characterized by a mutation in a gene selected from the group of KRAS, EGFR, BRAF and/or TP53 for group n, particularly wherein the mutation of KRAS is a KRAS G12 or a KRAS Q61 mutation.

In certain embodiments of the fourth aspect, the cancer is characterized by a mutation in a gene selected from the group of KRAS, EGFR, BRAF and/or TP53 for group o, particularly wherein the mutation of KRAS is a KRAS G12 or a KRAS Q61 mutation.

In certain embodiments, the cancer is characterized by a mutation in a gene selected from the group of KRAS for group a, particularly wherein the mutation of KRAS is a KRAS G12 mutation.

In certain embodiments, the cancer is characterized by a mutation in a gene selected from the group of TP53 for group b.

In certain embodiments, the cancer is characterized by a mutation in a gene selected from the group of KRAS for group d, particularly wherein the mutation of KRAS is a KRAS G12 mutation.

In certain embodiments, the cancer is characterized by a mutation in a gene selected from the group of KRAS and/or EGFR for group e, particularly wherein the mutation of KRAS is a KRAS G12 mutation.

In certain embodiments, the cancer is characterized by a mutation in a gene selected from the group of KRAS and/or EGFR for group f, particularly wherein the mutation of KRAS is a KRAS G12 or a KRAS Q61 mutation.

In certain embodiments, the cancer is characterized by a mutation in a gene selected from the group of TP53 for group i.

In certain embodiments, the cancer is characterized by a mutation in a gene selected from the group of KRAS and/or EGFR, particularly wherein the mutation of KRAS is a KRAS G12 mutation.

In certain embodiments, the cancer is characterized by a mutation in a gene selected from the group of EGFR and/or TP53 for group k.

In certain embodiments, the cancer is characterized by a mutation in a gene selected from the group of KRAS, EGFR, and/or TP53 for group n, particularly wherein the mutation of KRAS is a KRAS G12 mutation.

In certain embodiments, the cancer is characterized by a mutation in a gene selected from the group of KRAS, EGFR and/or TP53 for group o, particularly wherein the mutation of KRAS is a KRAS G12 mutation.

A further aspect of the invention relates to an agent selected from
a TCR polypeptide,
an isolated nucleic acid sequence encoding the TCR polypeptide, or
an isolated autologous T cell comprising the TCR polypeptide and/or the nucleic acid sequence;
for use in treatment of cancer,
wherein the TCR polypeptide comprises a CDR3 alpha sequence and a CDR3 beta sequence, wherein the CDR3 alpha sequence and the CDR3 beta sequence are identical to the sequences given below, or with one amino acid substitution per CDR3 sequence, wherein the CDR3 alpha sequence is selected from the sequences SEQ ID NO 007-009 or SEQ ID NO 436-439, and the CDR3 beta sequence is selected from the sequences SEQ ID NO 001-006 or SEQ ID NO 432-435,
wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or 99% sequence identity to said sequences, wherein the V alpha sequence is SEQ ID NO 025, the JC alpha sequence is SEQ ID NO 026, the V beta sequence is SEQ ID NO 018, and the JC beta sequence is SEQ ID NO 019,
wherein the agent is administered to a patient characterized by the HLA type HLA-A*02:01,
and wherein said cancer is characterized by a mutation in a gene of the EGFR-Raf-Ras pathway.

In certain embodiments of this further aspect, said mutation in a gene of the EGFR-Raf-Ras pathway is a mutation in KRAS and/or EGFR.

In certain embodiments of this further aspect, said mutation in a gene of the EGFR-Raf-Ras pathway is a KRAS G12 mutation.

### Medical Treatment, Dosage Forms and Salts

Similarly, within the scope of the present invention is a method or treating cancer in a patient in need thereof, comprising administering to the patient a the TCR according to the third aspect, the nucleic acid sequence according to the fourth aspect, or the isolated autologous T cell according to the fifth aspect.

Similarly, a dosage form for the prevention or treatment of cancer is provided, comprising a the TCR according to the third aspect, the nucleic acid sequence according to the fourth aspect, or the isolated autologous T cell according to the fifth aspect.

### Pharmaceutical Compositions and Administration

Another aspect of the invention relates to a pharmaceutical composition comprising the TCR according to the third aspect, the nucleic acid sequence according to the fourth aspect, or the isolated autologous T cell according to the fifth aspect.

In certain embodiments of the invention, the compound of the present invention is typically formulated into pharmaceutical dosage forms to provide an easily controllable dosage of the drug and to give the patient an elegant and easily manageable product.

The pharmaceutical composition can be formulated for parenteral administration, for example by i.v. infusion.

### Method of Manufacture and Method of Treatment According to the Invention

The invention further encompasses, as an additional aspect, the use of the TCR according to the third aspect, the nucleic acid sequence according to the fourth aspect, or the isolated autologous T cell according to the fifth aspect, as specified in detail above, for use in a method of manufacture of a medicament for the treatment or prevention of cancer.

Similarly, the invention encompasses methods of treatment of a patient having been diagnosed with a disease associated with cancer. This method entails administering to the patient the TCR according to the third aspect, the nucleic acid sequence according to the fourth aspect, or the isolated autologous T cell according to the fifth aspect.

The specification further comprises the following items:

### Items:

1. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence,
   wherein, for group a, the CDR3 alpha sequence is selected from the sequences SEQ ID NO 007-009 or SEQ ID NO 436-439, and the CDR3 beta sequence is selected from the sequences SEQ ID NO 001-006 or SEQ ID NO 432-435.
2. The isolated TCR polypeptide according to item 1, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group a, the V alpha sequence is SEQ ID NO 025, the JC alpha sequence is SEQ ID NO 026, the V beta sequence is SEQ ID NO 018, and the JC beta sequence is SEQ ID NO 019.
3. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence,
   wherein, for group b, the CDR3 alpha sequence is selected from the sequences SEQ ID NO 031-032, and the CDR3 beta sequence is selected from the sequences SEQ ID NO 027-030.
4. The isolated TCR polypeptide according to item 3, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group b, the V alpha sequence is SEQ ID NO 046, the JC alpha sequence is SEQ ID NO 047, the V beta sequence is SEQ ID NO 040, and the JC beta sequence is SEQ ID NO 041.
5. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence,
   wherein, for group c, the CDR3 alpha sequence is selected from the sequences SEQ ID NO 052-054, and the CDR3 beta sequence is selected from the sequences SEQ ID NO 048-051.
6. The isolated TCR polypeptide according to item 5, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group c, the V alpha sequence is SEQ ID NO 068, the JC alpha sequence is SEQ ID NO 069, the V beta sequence is SEQ ID NO 061, and the JC beta sequence is SEQ ID NO 062.
7. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group d, the CDR3 alpha sequence is selected from the sequence SEQ ID NO 073, and the CDR3 beta sequence is selected from the sequences SEQ ID NO 070-072.
8. The isolated TCR polypeptide according to item 7, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group d, the V alpha sequence is SEQ ID NO 084, the JC alpha sequence is SEQ ID NO 085, the V beta sequence is SEQ ID NO 079, and the JC beta sequence is SEQ ID NO 080.
9. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group e, the CDR3 alpha sequence is selected from the sequence SEQ ID NO 100 or SEQ ID NO 455-459, and the CDR3 beta sequence is selected from the sequences SEQ ID NO 086-099 or SEQ ID NO 450-454.
10. The isolated TCR polypeptide according to item 9, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group e, the V alpha sequence is SEQ ID NO 122, the JC alpha sequence is SEQ ID NO 0123, the V beta sequence is SEQ ID NO 117, and the JC beta sequence is SEQ ID NO 118.
11. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group f, the CDR3 alpha sequence is selected from the sequences SEQ ID NO 131-132 or SEQ ID NO 472-473, and the CDR3 beta sequence is selected from the sequences SEQ ID NO 124-130 or SEQ ID NO 470-471.
12. The isolated TCR polypeptide according to item 11, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group f, the V alpha sequence is SEQ ID NO 148, the JC alpha sequence is SEQ ID NO 149, the V beta sequence is SEQ ID NO 142, and the JC beta sequence is SEQ ID NO 143.
13. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group g, the CDR3 alpha sequence is selected from the sequence SEQ ID NO 156, and the CDR3 beta sequence is selected from the sequences SEQ ID NO 150-155.
14. The isolated TCR polypeptide according to item 13, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group g, the V alpha sequence is SEQ ID NO 170, the JC alpha sequence is SEQ ID NO 171, the V beta sequence is SEQ ID NO 165, and the JC beta sequence is SEQ ID NO 166.
15. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group h, the CDR3 alpha sequence is selected from the sequences SEQ ID NO 175-176, and the CDR3 beta sequence is selected from the sequences SEQ ID NO 172-174.
16. The isolated TCR polypeptide according to item 15, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group h, the V alpha sequence is SEQ ID NO 188, the JC alpha sequence is SEQ ID NO 189, the V beta sequence is SEQ ID NO 182, and the JC beta sequence is SEQ ID NO 183.
17. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group i, the CDR3 alpha sequence is selected from the sequences SEQ ID NO 193-194, and the CDR3 beta sequence is selected from the sequences SEQ ID NO 190-192.
18. The isolated TCR polypeptide according to item 17, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group i, the V alpha sequence is SEQ ID NO 206, the JC alpha sequence is SEQ ID NO 207, the V beta sequence is SEQ ID NO 200, and the JC beta sequence is SEQ ID NO 201.
19. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence,
   wherein, for group j, the CDR3 alpha sequence is selected from the sequences SEQ ID NO 212-213 or SEQ ID NO 513, and the CDR3 beta sequence is selected from the sequences SEQ ID NO 208-211 or SEQ ID NO 478.
20. The isolated TCR polypeptide according to item 19, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group j, the V alpha sequence is SEQ ID NO 226, the JC alpha sequence is SEQ ID NO 227, the V beta sequence is SEQ ID NO 220, and the JC beta sequence is SEQ ID NO 221.
21. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group k, the CDR3 alpha sequence is selected from the sequences SEQ ID NO 232-235, and the CDR3 beta sequence is selected from the sequences SEQ ID NO 228-231.
22. The isolated TCR polypeptide according to item 21, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group k, the V alpha sequence is SEQ ID NO 250, the JC alpha sequence is SEQ ID NO 251, the V beta sequence is SEQ ID NO 242, and the JC beta sequence is SEQ ID NO 243.
23. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group l, the CDR3 alpha sequence is selected from the sequences SEQ ID NO 261-269 or SEQ ID NO 484-486, and the CDR3 beta sequence is selected from the sequences SEQ ID NO 252-260 or SEQ ID NO 481-483.
24. The isolated TCR polypeptide according to item 23, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group l, the V alpha sequence is SEQ ID NO 294, the JC alpha sequence is SEQ ID NO 295, the V beta sequence is SEQ ID NO 281, and the JC beta sequence is SEQ ID NO 282.
25. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence,
   wherein, for group m, the CDR3 alpha sequence is selected from the sequences SEQ ID NO 302-307 or SEQ ID NO 496-498, and the CDR3 beta sequence is selected from the sequences SEQ ID NO 296-301 or SEQ ID NO 493-495.
26. The isolated TCR polypeptide according to item 25, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group m, the V alpha sequence is SEQ ID NO 326, the JC alpha sequence is SEQ ID NO 327, the V beta sequence is SEQ ID NO 316, and the JC beta sequence is SEQ ID NO 317.
27. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group n, the CDR3 alpha sequence is selected from the sequences SEQ ID NO 334-339, and the CDR3 beta sequence is selected from the sequences SEQ ID NO 328-333.
28. The isolated TCR polypeptide according to item 27, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group n, the V alpha sequence is SEQ ID NO 358, the JC alpha sequence is SEQ ID NO 359, the V beta sequence is SEQ ID NO 348, and the JC beta sequence is SEQ ID NO 349.
29. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence, wherein, for group o, the CDR3 alpha sequence is selected from the sequences SEQ ID NO 363-365, and the CDR3 beta sequence is selected from the sequences SEQ ID NO 360-362.
30. The isolated TCR polypeptide according to item 29, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group o, the V alpha sequence is SEQ ID NO 378, the JC alpha sequence is SEQ ID NO 379, the V beta sequence is SEQ ID NO 371, and the JC beta sequence is SEQ ID NO 372.
31. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence,
   wherein, for group p, the CDR3 alpha sequence is selected from the sequences SEQ ID NO 391-401 or SEQ ID NO 507-508, and the CDR3 beta sequence is selected from the sequences SEQ ID NO 380-390 or SEQ ID NO 505-506.
32. The isolated TCR polypeptide according to item 29, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein for group p, the V alpha sequence is SEQ ID NO 430, the JC alpha sequence is SEQ ID NO 431, the V beta sequence is SEQ ID NO 415, and the JC beta sequence is SEQ ID NO 416.
33. The isolated TCR polypeptide according to any one of items 1 to 32, wherein the TCR polypeptide comprises a CDR3 alpha sequence and a CDR3 beta sequence, wherein the CDR3 alpha sequence and the CDR3 beta sequence are identical to the sequences given, or with one amino acid substitution per CDR3 sequence.
34. The isolated TCR polypeptide according to any one of items 1 to 32, wherein the TCR polypeptide comprises a CDR3 alpha sequence and a CDR3 beta sequence, wherein the CDR3 alpha sequence and the CDR3 beta sequence are identical to the sequences given, without any amino acid substitution.
35. The isolated TCR polypeptide according to any one of items 1 to 34, wherein the CRD3 alpha sequence and the CDR3 beta sequence are identified in the same row of tables 1-16.
36. The isolated TCR polypeptide according to any one of items 1 to 35, wherein said substitutions are selected according to the substitution rules given below,
   wherein the substitution rules are:
      glycine (G) and alanine (A) are interchangeable; valine (V), leucine (L), and isoleucine (I) are interchangeable, A and V are interchangeable;
      tryptophan (W) and phenylalanine (F) are interchangeable, tyrosine (Y) and F are interchangeable;
      serine (S) and threonine (T) are interchangeable;
      aspartic acid (D) and glutamic acid (E) are interchangeable
      asparagine (N) and glutamine (Q) are interchangeable; N and S are interchangeable; N and D are interchangeable; E and Q are interchangeable;
      methionine (M) and Q are interchangeable;
      cysteine (C), A and S are interchangeable;
      proline (P), G and A are interchangeable;
      arginine (R) and lysine (K) are interchangeable.
37. A library of TCR polypeptides, said library comprising at least two TCR polypeptides from different clusters a-p as described in any one of the preceding items.
38. An isolated nucleic acid sequence encoding the TCR polypeptide according to any one of the preceding items 1 to 36.
39. A library of isolated nucleic acid sequences encoding TCR polypeptides, said library comprising at least two isolated nucleic acid sequences each encoding a TCR polypeptides from a different cluster a-p as described in any one of the preceding items 1 to 36.
40. An isolated autologous T cell comprising a TCR polypeptide according to any one of items 1 to 36, and/or a nucleic acid sequence according to item 38.
41. The isolated autologous T cell according to item 40, wherein the isolated autologous T cell is a recombinant T cell.
42. A library of isolated autologous T cells comprising TCR polypeptides, said library comprising at least two isolated autologous T cells each comprising a TCR polypeptide from a different cluster a-p as described in any one of the preceding items 1 to 36.
43. An agent selected from the TCR polypeptide according to any one of items 1 to 36, the nucleic acid sequence according to item 38, or the isolated autologous T cell according to items 40 or 41 for use in treatment of cancer.
44. The agent for use according to item 43, wherein agent is administered to a patient characterized by the following HLA-type:
   a. HLA-A*02:01 for group a; or
   b. HLA-B*08:01 and/or HLA-C*07:01 for group b; or
   c. HLA-A*02:01 for group c; or
   d. HLA-A*02:01 for group d; or
   e. HLA-B*15:01 for group e; or
   f. HLA-A*02:01 for group f; or
   g. HLA-B*08:01 for group g; or
   h. HLA-B*07:02 for group h; or
   i. HLA-A*01:01 and/or HLA-B*08:01 and/or HLA-C*07:01 for group i; or
   j. HLA-A*02:01 for group j; or
   k. HLA-A*02:01 for group k.
45. The agent for use according to any one of items 43 or 44, wherein said cancer is a solid tumor.
46. The agent for use according to any one of items 43 or 44, wherein said cancer is selected from lung cancer, pancreatic cancer, colon cancer, and breast cancer.
47. The agent for use according to any one of items 43 or 44, wherein said cancer is selected from the group of Bladder Urothelial Carcinoma, Breast invasive carcinoma, Cervical squamous cell carcinoma and endocervical adenocarcinoma, Cholangiocarcinoma, Colon adenocarcinoma, Lymphoid Neoplasm Diffuse Large B-cell Lymphoma, Esophageal carcinoma, Glioblastoma multiforme, Head and Neck squamous cell carcinoma, Kidney Chromophobe, Kidney renal papillary cell carcinoma, Acute Myeloid Leukemia, Brain Lower Grade Glioma, Lung adenocarcinoma, Lung squamous cell carcinoma, Mesothelioma, Ovarian serous cystadenocarcinoma, Pancreatic adenocarcinoma, Rectum adenocarcinoma, Sarcoma, Skin Cutaneous Melanoma, Stomach adenocarcinoma, Testicular Germ Cell Tumors, Thyroid carcinoma, Uterine Corpus Endometrial Carcinoma, Uterine Carcinosarcoma.
48. The agent for use according to any one of items 43 to 47, wherein said cancer is characterized by a mutation in a gene selected from the group of
   a. KRAS, EGFR, and/or TP53 for group a, particularly wherein the mutation of KRAS is a KRAS G12 mutation; or
   b. KRAS, FGFR, and/or TP53 for group b, particularly wherein the mutation of KRAS is a KRAS Q61 mutation; or
   c. KRAS, and/or EGFR for group c, particularly wherein the mutation of KRAS is a KRAS G12 or a KRAS Q61 mutation; or
   d. KRAS and/or TP53 for group d, particularly wherein the mutation of KRAS is a KRAS G12 mutation; or
   e. KRAS, EGFR and/or BRAF for group e, particularly wherein the mutation of KRAS is a KRAS G12 or a KRAS Q61 mutation; or
   f. KRAS, EGFR and/or BRAF for group f, particularly wherein the mutation of KRAS is a KRAS G12 or a KRAS Q61 mutation; or
   g. TP53 for group g; or
   h. KRAS, EGFR, BRAF and/or TP53 for group h, particularly wherein the mutation of KRAS is a KRAS G12 or a KRAS Q61 mutation; or
   i. EGFR, FGFR, and/or TP53 for group i; or
   j. KRAS, EGFR and/or TP53, particularly wherein the mutation of KRAS is a KRAS G12 mutation; or
   k. KRAS, EGFR, BRAF and/or TP53 for group k, particularly wherein the mutation of KRAS is a KRAS G12 or a KRAS Q61 mutation; or
   n. KRAS, EGFR, BRAF and/or TP53 for group n, particularly wherein the mutation of KRAS is a KRAS G12 or a KRAS Q61 mutation; or
   o. KRAS, EGFR, BRAF and/or TP53 for group o, particularly wherein the mutation of KRAS is a KRAS G12 or a KRAS Q61 mutation.
49. The agent for use according to any one of items 7 to 9, wherein said cancer is characterized by a mutation in a gene selected from the group of
   a. KRAS for group a, particularly wherein the mutation of KRAS is a KRAS G12 mutation; or
   b. TP53 for group b; or
   d. KRAS for group d, particularly wherein the mutation of KRAS is a KRAS G12 mutation; or
   e. KRAS and/or EGFR for group e, particularly wherein the mutation of KRAS is a KRAS G12 mutation; or
   f. KRAS and/or EGFR for group f, particularly wherein the mutation of KRAS is a KRAS G12 or a KRAS Q61 mutation; or
   i. TP53 for group i; or
   j. KRAS and/or EGFR, particularly wherein the mutation of KRAS is a KRAS G12 mutation; or
   k. EGFR and/or TP53 for group k; or
   n. KRAS, EGFR, and/or TP53 for group n, particularly wherein the mutation of KRAS is a KRAS G12 mutation; or
   o. KRAS, EGFR and/or TP53 for group o, particularly wherein the mutation of KRAS is a KRAS G12 mutation.

The invention is further illustrated by the following examples and figures, from which further embodiments and advantages can be drawn. These examples are meant to illustrate the invention but not to limit its scope.

### DESCRIPTION OF THE FIGURES

FIG. 1: Schema of tsTCR foot print table. From left to right the tumour specific TCR footprint table comprises the following elements per TCR clonotype: CDR3β amino acid sequence, CDR3α amino acid sequence, frequency of CDR3β sequence in percent, V-segment ID of p chain, J-segment ID of p chain, V-segment ID of a chain, J-segment ID of a chain, HLA-type(s) in 4-digit resolution (class I or II), a set of marker genes in several columns with respective expression rates per clonotype.

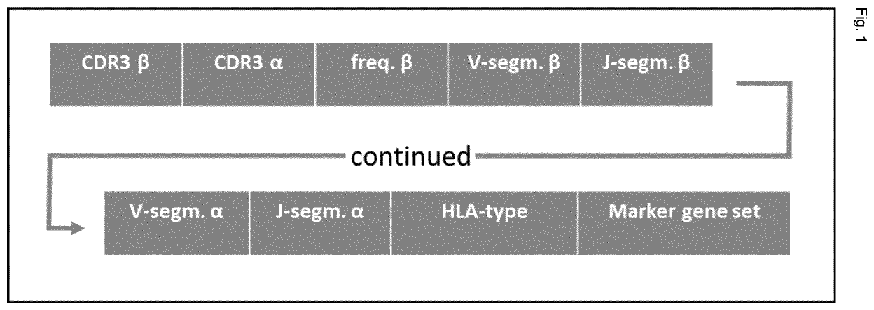

FIG. 2: TCR clustering schema, part I. These are the steps for a TCR repertoire analysis in tumour and non-tumour condensed finally into a tumour-specific TCR foot print table

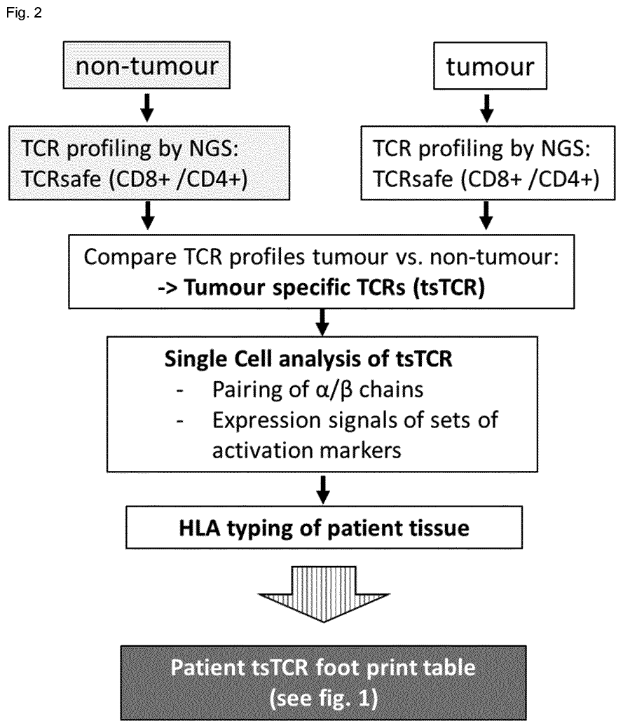

FIG. 3: TCR clustering schema, part II. For 2, 3, or more different patients one table represents a TCR cluster with closely related TCRs. The columns 1-13 and NN are described below. 1: An arbitrary patient ID. 2-3: Amino acid sequences of both CDR3 chains. 4: The ratio between TCR-clonotype frequencies in tumour versus adjacent non-tumour tissue. 5: The frequency of respective TCR in tumour. 6-9: V/J segments of both chains. 10: HLA type(s) (I/II) in 4-digit resolution. 11-13: Respective T-cell activation marker frequencies. They are measured with single-cell sequencing gene expression technology or, if applicable, with cell sorting technology and respective clonotype frequencies are derived from TCR sequencing data. NN: Any marker for T-cell activation might be used the same way.

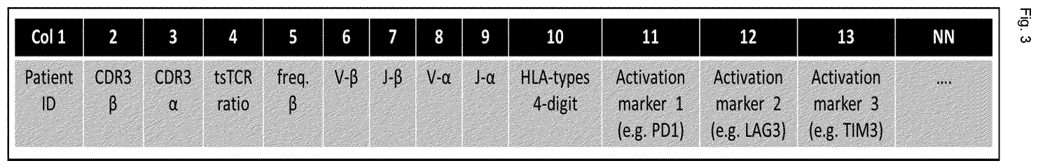

FIG. 4: Cell growth (proliferation, differentiation) and survival in healthy cells are controlled by external signals. The prototypic receptor tyrosine kinase (RTK) EGFR becomes activated through binding of epidermal growth factor (EGF) and transduces stimulatory signals from the cell membrane to the nucleus by activating the RAS-RAF-MEK-ERK pathway. Mutation-activated EGFR, RAS (e.g. KRASpG12-mutations), or RAF (e.g. BRAFpV600E) are constitutively active and produce stimulatory signals independent of external signals.

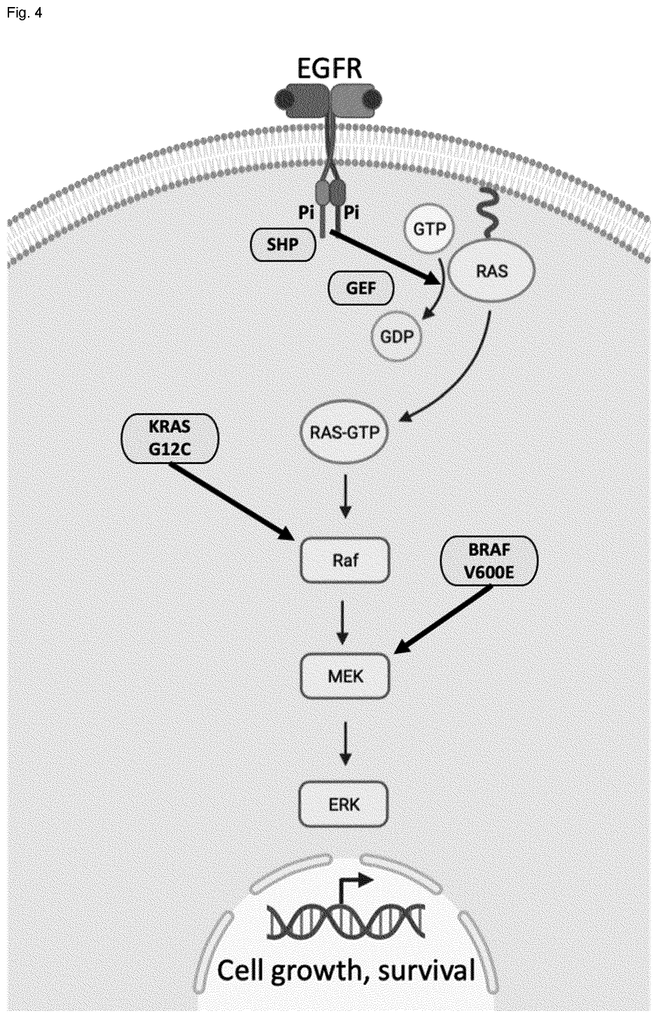

FIG. 5: CD8+ cells from a donor were depleted from their endogenous TCRs and transduced with three different TCRs from cluster a. The resulting TCR-T cells were tested against four HLA-A*02:01-positive NSCLC cell lines by IFN-γ Elispot assay. The cell lines NCI-H1792 and MZ-LC-16, recognized by all three TCR-T cells, have in common that they carry a KRASpG12C mutation. MOR/CPR and NCI-H661 have no mutation in KRAS. In TCR cluster a, tumors of seven of nine patients tested are positive for KRASpG12-mutations (Table 17).

### EXAMPLES

### A.1: Preparation of Non-Small Cell Lung Cancer (NSCLC) Tumor- and Normal Lung Tissue Specimen and TCRSafe Analysis

Each tumor specimen is dissected free of surrounding normal tissue and necrotic areas. Approx. 1 g cubes from tumor and normal lung tissue are cut into small chunks measuring about 2-3 mm in each dimension. Sliced tumor (and also non-tumor) biopsies are subjected to a commercial mechanical/enzymatic tissue dissociation system (GentleMACS, Miltenyi Biotec, Bergisch-Gladbach, Germany), using the Tumor Dissociation Kit (Miltenyi Biotech) and following the manufacturer's instructions. After GentleMACS disaggregation, cell suspensions are passed through 70-μm cell strainers. Aliquots of tumor- and lung cells are taken and cryopreserved in 10% DMSO (Sigma-Aldrich) and 90% FCS (Life Technologies) for later use. The remaining cell suspensions are subjected to density gradient centrifugation using a 40%/80% step gradient of Percoll® (GE Healthcare Europe GmbH) in PBS/RPMI 1640. T-lymphocytes are harvested from interphases and washed in complete medium (RPMI 1640, Lonza). Subsequently, tumor-infiltrating T-lymphocytes (TIL) and lymphocytes from normal lung tissue are placed in 24-well tissue culture plates with 2 mL of recovery medium (RM) at a concentration of 0.5×106 cells/ml. RM is RPMI 1640 supplemented with 25 mM HEPES pH 7.2 and L-glutamine (Lonza), 100 IU/mL penicillin, 100 mg/mL streptomycin, and 50 mM beta-mercaptoethanol (ThermoFisher Scientific, Waltham, Massachusetts, USA), supplemented with 10% autologous human serum. Plates are placed in a humidified 37° C. incubator with 5% CO2 and cultured overnight. The next day, cells are harvested and pooled from the TIL- and normal lung cultures and the following subpopulations isolated via FACS:
CD4+ T cells
CD8+ T cells
PD1+ cells (from TIL only)
PD1− negative cells (from TIL only)

From subpopulations, genomic DNA is extracted and subjected to TCRsafe analysis (as disclosed in WO 2014/096394 A1). The resulting T-cell clonotype frequencies are compared among subpopulations and tumor-specific clonotypes identified as detailed in WO 2017/025564 A1.

All subsequent steps for these examples refer to CD8+ T-cells isolated from tumour and non-tumour tissues as described above.

Patient IDs designated as “P” followed by a number relate to non-small cell lung cancer patients.

Patient IDs designated as “PANC” followed by a number relate to pancreatic cancer patients.

### a.2: T Cell Receptor (TCR) α/β Pairing Using 10× Genomics High Throughput Single-Cell Sequencing

Starting from TIL single-cell suspensions, 5000-10000 T-cells are subjected to high throughput single-cell RNASeq analysis using the 10× Genomics Chromium Next GEM Single Cell V(D)J Reagent Kit in combination with the Chromium Single Cell V(D)J Enrichment Kit (Human). 10× Genomics® GemCode™ Technology disperses thousands of individual cells into Gel Bead-in-EMulsion (GEM) droplets. GEM-captured single cells are lysed and upon GEM-solution, barcoded primers attached to the beads, oligos, master mix, and lysed cell components are mixed, and through RT-PCR, full-length oligo-dT-primed cDNA-libraries are generated. First-strand cDNA synthesis by using a template switch mechanism is completed including the barcoded sequence attached to the beads. All cDNA molecules within a single GEM are labelled with the same barcode. GEMs are broken down and further library preparations are continued as bulk reactions. After cDNA-clean up, the Chromium Single Cell V(D)J Enrichment Kit effectively amplifies TCR sequences and generates sequencing libraries compatible with Illumina sequencing. In combination with whole cDNA amplification, Illumina sequencing reveals for each single T cell analyzed the paired α/βTCR sequences and the corresponding whole transcriptome per cell. Both kits, the 10× Genomics Chromium Next GEM Single Cell V(D)J Reagent Kit and the Chromium Single Cell V(D)J Enrichment Kit (Human) are used according to the manufacturer's recommendations.

Nucleotide sequences identical between TCRs described under A.1 above and TCRs from the single-cell VDJ pairing are used to establish the full annotation of TCRs with respect to alpha- and beta chains, frequencies and tumour specificity.

### A.3: Clustering of TCRs (TCRpolyClust)

As described in the schema of TCRpolyClust method once the combination of A.1 and A.2 is established a subsequent TCR cluster analysis identifies patients with common TCRs and matching HLA-types enabling the screening for shared tumor antigens.

Correlation of Known Tumor Driver Mutations with Common Tumor Specific TCRs

There is a well-known set of tumor mutations (KRAS, EGFR etc.) which are routinely screened by sequencing techniques and use of available oncology panels of primers (e.g. QlAseq Targeted DNA Panel, AmpliSeq for Illumina Focus Panel, etc.). While there was little success so far to find public tumor antigens under this set of tumor mutations there is strong evidence that existence of common tumor specific TCRs is well correlated with occurrence of dedicated and frequent tumor mutations. Therefore, patients may be well selected for TCR-T therapies via common TCRs once they belong to the respective HLA-type and are carriers of the respective mutation.

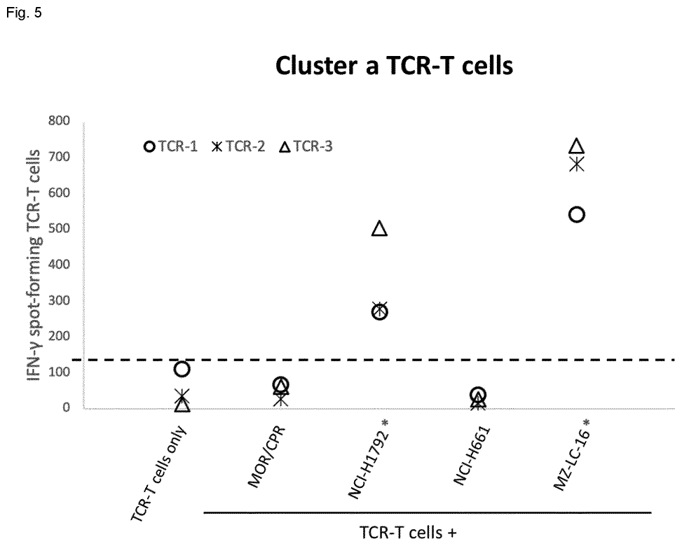

TCR-T cells transduced with three TCRs of cluster-a showed almost identical response patterns as shown in FIG. 5: While the spontaneous IFN-γ secretion (TCR-T cells only) was low to moderate (delimited by the dashed line), all three TCR-T cells specifically secreted high amounts of IFN-γ in response to the NSCLC cell lines NCI-H1792 and MZ-LC-16—calculated as number of IFN-γ spot-producing cells per number of TCR T cells per test reaction. In co-cultures with tumor cell lines NCI-H661 and MOR/CPR, no IFN-γ response was detected. NGS analyses of the tumor mutation profiles of the cell lines revealed that the only mutation the recognized cell lines NCI-H1792 and MZ-LC-16 have in common is the oncogenic driver mutation KRASpG12C. This mutation is absent in the two tumor lines not recognized by the TCR-T cells. Strikingly, in TCR cluster-a, tumors of seven of nine patients tested are positive for different KRASpG12-mutations (Table 17).

The frequencies of common mutations along most of the TCR clusters a-o are summarized in table 17, the respective frequencies of these mutations in all relevant cancers is shown in table 18 and 19. The numbers in table 17 were derived from mutational screens (onco-panels) of the respective patient tumors.

### Materials & Methods:

T lymphocytes isolated from a buffy coat of a healthy donor were depleted from their endogenous TCRs by CRISPR/CAS9 gene knockout (KO). Subsequently, the T cells were transduced by means of retroviral transduction with three recombinant TCRs from TCR cluster a. Following expansion in vitro and confirmation (by FACS) that the recombinant TCRs were expressed on the surfaces of the cells, the resulting TCR-T cells were tested for recognition of HLA-A02-positive NSCLC cell lines MOR/CPR, NCI-H1792, NCI-H661, and MZ-LC-16. We focused on recognition of HLA-A02-positive tumor cells, because the patients in cluster a showed shared expression of HLA-A*02:01. A 96-well format IFN-γ ELISpot assay was performed to test whether the TCR-T cells secrete IFN-γ in response to co-culture with the cell lines. The spontaneous IFN-γ release by the TCR-T cells served as background control. All reactions were carried out in duplicates. Results (FIG. 5) are shown as average number of IFN-γ spot-producing cells per duplicate.

Synthesis, Cloning, and Ectopic Expression of Cluster TCRs in T Cells Isolated from Autologous Patients

or Healthy Donors Paired cluster TCRs are codon-optimized, synthesized, and cloned as bicistronic chimeric constructs (βTCR-VDJ-mC_P2A-element_αTCR-VJ-mC; mC represent murine constant domains) into retroviral (or comparable) expression vectors for transduction of autologous or allogeneic T cells from blood of the respective patients or healthy donors. Recipient T cells are pretreated with CRISPR/Cas9 to knock-out endogenous TCRs to prevent off-target immune reactions mediated by mixed TCR-dimers (endogenous x exogenous chains, in autologous and allogeneic settings) or allo-responses by endogenous TCRs (in allogeneic settings). Said chimeric (c) TCR-recombinant T cells are expanded in vitro and applied to functional experiments such as recognition of autologous tumor cells (if available), allogeneic tumor cell lines, and/or antigen-screenings as described below.

### Targeted Approaches for the Screening of Shared Tumor Antigens

Comparative whole-exome (WES) and whole transcriptome (WTS) sequencing of tumor- and corresponding normal tissue genomic and total-RNA including samples from all patients of a respective TCR cluster are applied to identify shared neoantigens (SNV, MNV, InDels, fusion gene products, structural alterations), aberrantly expressed canonical genes (cancer/germline- and overexpressed antigens), and aberrantly expressed and translated non-canonical transcripts (dark matter transcripts or cryptic transcripts). Candidates of all categories are then tested for recognition by the cTCR-transduced recombinant T cells. Antigen formats are either expression plasmids encoding full-length antigen-cDNAs or tandem minigenes (TMGs) encoding only the peptide-coding regions with immunogenic potential of the candidate antigens. Both formats are tested by co-transfection of antigen- and HLA-cDNA-encoding plasmids in 293T- or COS-7 cells and subjecting the transfectants to recognition testing by the T cells in IFN-γ ELISpot assays. Alternatively, antigenic peptide candidates can be predicted for binding to the relevant HLA alleles using public prediction algorithms (IEDB, NetMHC), the peptides synthesized and pulsed onto HLA-matched antigen-presenting cells. The latter are then subjected to ELISpot assays testing their recognition by the recombinant T cells.

Tumor cDNA-Expression-Library Screening Approaches

The targeted identification of antigen candidates may not be equally effective for all antigen categories. For example, while the screening for non-synonymous somatic mutations of tumor cells using whole-exome- and -transcriptome sequencing is sensitive, highly reproducible, and produces a quantitative list of potential neoantigens, the identification of cryptic translatable transcripts (dark matter antigens) is less efficient due to a general lack of specific traits to identify them reliably. This dilemma can be solved by probing the complete transcriptome of tumor cells by cNA-expression-library screening approaches. Either from sorted autologous tumor cells or from HLA-matched tumor cell lines, shown before to be recognized by cTCR-transduced T cells, cDNA-expression libraries generated from total-RNA are co-expressed with appropriate HLA-alleles in antigen-presenting cells (293T- or COS-7 cells). Transfectants are then tested for recognition by cTCR-transduced T cells via ELISpot assays. To have a chance to get expression even of rare transcripts after transfection, the screening procedure requires a high throughput approach testing a highly fractionated cDNA-library. For this purpose, a cDNA-library is produced consisting e.g. of 2000 pools of 100 cDNAs per well prepared in a 96-well plate format. Transfections and ELISpot assays using the cTCR-transduced T cells as effector cells are conducted in this 96-well format and from recognized pools of 100, step-wise reduction of pools (e.g. 10 cDNAs/pool and well, cDNA-clones/pool and well) and testing will result in the selection of antigen-encoding cDNA-clones.

Because a sufficiently pure and large enough population of viable autologous tumor cells for RNA-isolation and cDNA-library preparation can be obtained only for a minority of patients, a pre-screening for recognition of type-matched tumor cell lines can be conducted. The cell lines can either be selected for shared expression of HLA alleles or be transduced with HLAs of interest. Recognized cell lines are used as proof for the existence of the common antigen and as sources for RNA-extraction and cDNA-library generation.

TABLE 17
Per cluster (a-o) the table summarizes the total number of patients in each respective cluster
(A) and the number of patients with tumors analyzed for recurrent mutations (B). Six genes
with recurrent mutations were detected in the tumors, columns C-H depict how often mutations
in these genes were found. The mutated genes in columns C-G are involved in the same singnaling
pathway and mutations can be expected to have overlapping effects.

| | | A | B | | | | | | |
|---|---|---|---|---|---|---|---|---|---|
| | | number | number | | | | | | |
| cancer | | of | pts with | C | D | E | F | G | |
| types | | patients | mutation | KRAS | KRAS | EGFR- | BRAF- | FGFR- | H |
| analyzed | clusters | (pts) | analysis | G12-m | Q61-m | m | m | m | TP53-m |
| NSCLC, | Cluster a | 25 | 9 | 7 | | 1 | | | 1 |
| PANC | | | | | | | | | |
| NSCLC | Cluster b | 19 | 6 | | 2 | | | 1 | 3 |
| NSCLC | Cluster c | 14 | 4 | 2 | 1 | 1 | | | |
| NSCLC | Cluster d | 20 | 7 | 4 | | | | | 2 |
| NSCLC, | Cluster e | 27 | 14 | 5 | 2 | 4 | 1 | | |
| PANC | | | | | | | | | |
| NSCLC | Cluster f | 26 | 14 | 5 | 3 | 4 | 1 | | |
| NSCLC | Cluster g | 3 | 1 | | | | | | 1 |
| NSCLC | Cluster h | 12 | 8 | 2 | 1 | 2 | 2 | | 1 |
| NSCLC | Cluster i | 13 | 7 | | | 1 | | 1 | 6 |
| NSCLC, | Cluster j | 20 | 9 | 4 | | 4 | | | 1 |
| PANC | | | | | | | | | |
| NSCLC | Cluster k | 53 | 20 | 9 | 2 | 7 | 1 | | 4 |
| NSCLC | Cluster n | 55 | 20 | 9 | 1 | 5 | 1 | | 3 |
| NSCLC | Cluster o | 25 | 11 | 3 | 1 | 3 | 1 | | 3 |

TABLE 18
Overview over the most frequent known tumor mutations
as listed in TCGA. The percentages refer to the respective
cancer types where the mutation is found in. The abbreviation
of cancer types is explained in table 19.

| | Percentages among cancer data sets in |
|---|---|
| Tumor mutation | TCGA* datasets (tumor types only) |
| BRAFpV600E | THCA: 283 / 490 (57.76%), SKCM: 200 / 470 |
| | (42.55%), COAD: 50 / 428 (11.68%), CHOL: 1 / |
| | 51 (1.96%), LUAD: 8 / 559 (1.43%), GBM: 4 / |
| | 374 (1.07%) |
| EGFRpE746_A750del | LUAD: 18 / 559 (3.22%) |
| EGFRpL858R | LUAD: 22 / 559 (3.94%), |
| KRASpG12A | LUAD: 17 / 559 (3.04%), READ: 3 / 155 |
| | (1.94%), COAD: 8 / 428 (1.87%), UCS: 1 / 57 |
| | (1.75%), UCEC: 8 / 512 (1.56%) |
| KRASpG12C | LUAD: 56 / 559 (10.02%), READ: 7 / 155 |
| | (4.52%), COAD: 10 / 428 (2.34%), UCS: 1 / 57 |
| | (1.75%), MESO: 1 / 80 (1.25%), |
| KRASpG12D | 7 |
| KRASpG12V | PAAD: 29 / 179 (16.20%), COAD: 38 / 428 |
| | (8.88%), LUAD: 39 / 559 (6.98%), UCS: 3 / 57 |
| | (5.26%), UCEC: 19 / 512 (3.71%), TGCT: 3 / |
| | 141 (2.13%), DLBC: 1 / 47 (2.13%), LAML: 3 / |
| | 144 (2.08%) |
| KRASpG13C | LUAD: 6 / 559 (1.07%) |
| KRASpQ61H | PAAD: 5 / 179 (2.8%), |
| TP53pR175H | READ: 13 / 155 (8.39%), COAD: 26 / 428 |
| | (6.07%), ESCA: 9 / 184 (4.89%), OV: 15 / 419 |
| | (3.58%), UCS: 2 / 57 (3.51%), STAD: 12 / 434 |
| | (2.76%), HNSC: 12 / 509 (2.36%), PAAD: 4 / |
| | 179 (2.23%), GBM: 7 / 374 (1.87%), BRCA: 18 / |
| | 969 (1.86%), SARC: 4 / 235 (1.70%), LGG: 8 / |
| | 513 (1.56%) |
| TP53pR196* | PAAD: 3 / 179 (1.68%), ESCA: 3 / 184 (1.63%), |
| | KICH: 1 / 66 (1.52%), READ: 2 / 155 (1.29%), |
| | HNSC: 6 / 509 (1.18%) |

*TCGA: The Cancer Genome Atlas. https://www.cancer.gov/ccg/research/genome-sequencing/tcga

TABLE 19
The abbreviation of most frequent cancer types.

| Cancer | |
|---|---|
| Symbol | Description |
| BLCA | Bladder Urothelial Carcinoma |
| BRCA | Breast invasive carcinoma |
| CESC | Cervical squamous cell carcinoma and endocervical |
| | adenocarcinoma |
| CHOL | Cholangiocarcinoma |
| COAD | Colon adenocarcinoma |
| DLBC | Lymphoid Neoplasm Diffuse Large B-cell Lymphoma |
| ESCA | Esophageal carcinoma |
| GBM | Glioblastoma multiforme |
| HNSC | Head and Neck squamous cell carcinoma |
| KICH | Kidney Chromophobe |
| KIRP | Kidney renal papillary cell carcinoma |
| LAML | Acute Myeloid Leukemia |
| LGG | Brain Lower Grade Glioma |
| LUAD | Lung adenocarcinoma |
| LUSC | Lung squamous cell carcinoma |
| MESO | Mesothelioma |
| OV | Ovarian serous cystadenocarcinoma |
| PAAD | Pancreatic adenocarcinoma |
| READ | Rectum adenocarcinoma |
| SARC | Sarcoma |
| SKCM | Skin Cutaneous Melanoma |
| STAD | Stomach adenocarcinoma |
| TGCT | Testicular Germ Cell Tumors |
| THCA | Thyroid carcinoma |
| UCEC | Uterine Corpus Endometrial Carcinoma |
| UCS | Uterine Carcinosarcoma |

### Sequences

TOR sequences are constructed as follows (from N to C terminus):

Block-V_{——}CDR3_{——}Block-J/C

| | |
|---|---|
| Block-V | Constant part of TCR-chain (alpha or beta) from start |
| | codon (M) to the variable CDR3 region |
| CDR3 | Complementarity-determining region 3 |
| Block-J/C | The constant region from CDR3 through J-segment to |
| | the respective C-element, ending with a stop (*) codon |

TABLE 1
Cluster ID a:

| | | CDR3 beta: | CDR3 alpha: | |
|---|---|---|---|---|
| SEQ-D | Pat-ID | Amino acid seq. | Amino acid seq. | HLA_A_I |
| a.1 | P82 | CASSNDGMNTEAFF | CAVLMDSNYQLIW | A*02:01 |
| | | (SEQ ID NO. 001) | (SEQ ID NO. 007) | |
| a.2 | P86 | CASSADGMNTEAFF | CAVLMDSNYQLIW | A*02:01 |
| | | (SEQ ID NO. 002) | (SEQ ID NO. 008) | |
| a.3 | P93 | CASSEDGMNTEAFF | CAALMDSNYQLIW | A*02:01 |
| | | (SEQ ID NO. 003) | (SEQ ID NO. 009) | |
| a.4 | P82 | CASSDDGMNTEAFF | | A*02:01 |
| | | (SEQ ID NO. 004) | | |
| a.5 | P82 | CASSTDGMNTEAFF | | A*02:01 |
| | | (SEQ ID NO. 005) | | |
| a.6 | P93 | CASSEDGMNTEAFF | | A*02:01 |
| | | (SEQ ID NO. 006) | | |
| a.7 | P101 | CASSGDGMNTEAFF (SEQ ID | CAVLMDSNYQLIW (SEQ ID | A*02:01 |
| | | NO. 432) | NO. 007) | |
| a.8 | PANC2 | CASSTDGMNTEAFF (SEQ ID | CAVLMDSNYQLIW (SEQ ID | A*02:01 |
| | | NO. 005) | NO. 007) | |
| a.9 | PANC7 | CASSESGENTEAFF (SEQ ID | CAPLDSNYQLIW (SEQ ID | A*02:01 |
| | | NO. 433) | NO. 436) | |
| a.10 | PANC7 | CASSESGENTEAFF (SEQ ID | CAAQDSNYQLIW (SEQ ID | A*02:01 |
| | | NO. 433) | NO. 437) | |
| a.11 | PANC9 | CASSPGENTEAFF (SEQ ID | CAAMDSNYQLIW (SEQ ID | A*02:01 |
| | | NO. 434) | NO. 438 | |
| a.12 | PANC9 | CASSPRGENTEAFF (SEQ ID | CAALDSNYQLIW (SEQ ID | A*02:01 |
| | | NO. 435) | NO. 439) | |

```
Cluster a is associated with HLA-A*02:01
Beta chain, TRB:
TRBV10-2*01, TRBJ1-1*01, TRBC1*01
Cluster a Block V
  (SEQ ID NO. 010)
ATGGGCACCAGGCTCTTCTTCTATGTGGCCCTTTGTCTGCTGTGGGCAGGACACAGGGATGCTGGAATCACCCAGAGCCC
AAGATACAAGATCACAGAGACAGGAAGGCAGGTGACCTTGATGTGTCACCAGACTTGGAGCCACAGCTATATGTTCTGGT
ATCGACAAGACCTGGGACATGGGCTGAGGCTGATCTATTACTCAGCAGCTGCTGATATTACAGATAAAGGAGAAGTCCCC
GATGGCTATGTTGTCTCCAGATCCAAGACAGAGAATTTCCCCCTCACTCTGGAGTCAGCTACCCGCTCCCAGACATCTGTGT
ATTTC
Seq-ID a.1b
(SEQ ID NO. 011)
TGCGCCAGCTCCAATGACGGGATGAACACTGAAGCTTTCTTT
Seq-ID a.2b
(SEQ ID NO. 012)
TGCGCCAGCAGCGCGGACGGGATGAACACTGAAGCTTTCTTT
Seq-ID a.3b
(SEQ ID NO. 013)
TGCGCCAGCAGTGAGGATGGCATGAACACTGAAGCTTTCTTT
Seq-ID a.4b
(SEQ ID NO. 014)
TGCGCCAGCAGTGACGACGGCATGAACACTGAAGCTTTCTTT
Seq-ID a.5b
  (SEQ ID NO. 015)
TGCGCCAGCAGTACCGACGGGATGAACACTGAAGCTTTCTTT
Seq-ID a.6b
  (SEQ ID NO. 016)
TGCGCCAGCAGTGAGGATGGCATGAACACTGAAGCTTTCTTT
Seq-ID a.7b
(SEQ ID NO. 440)
TGCGCCAGCAGTGGGGACGGAATGAACACTGAAGCTTTCTTT
Seq-ID a.8b
(SEQ ID NO. 015)
TGCGCCAGCAGTACCGACGGGATGAACACTGAAGCTTTCTTT
Seq-ID a.9b
  (SEQ ID NO. 441)
TGTGCCAGCAGTGAATCCGGAGAGAACACTGAAGCTTTCTTT
Seq-ID a.10b
(SEQ ID NO. 441)
TGTGCCAGCAGTGAATCCGGAGAGAACACTGAAGCTTTCTTT
Seq-ID a.11b
(SEQ ID NO. 442)
TGTGCCAGCAGTCCTGGGGAAAATACTGAAGCTTTCTTT
Seq-ID a.12b
  (SEQ ID NO. 443)
TGTGCCAGCAGCCCCAGGGGGGAGAACACTGAAGCTTTCTTT
Cluster a Block J/C
  (SEQ ID NO. 017)
GGACAAGGCACCAGACTCACAGTTGTAGAGGACCTGAACAAGGTGTTCCCACCCGAGGTCGCTGTGTTTGAGCCATCAGA
AGCAGAGATCTCCCACACCCAAAAGGCCACACTGGTGTGCCTGGCCACAGGCTTCTTCCCTGACCACGTGGAGCTGAGCT
GGTGGGTGAATGGGAAGGAGGTGCACAGTGGGGTCAGCACGGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCCGTCACCCAGATCGTCAGCGCCGAG
GCCTGGGGTAGAGCAGACTGTGGCTTTACCTCGGTGTCCTACCAGCAAGGGGTCCTGTCTGCCACCATCCTCTATGAGATC
CTGCTAGGGAAGGCCACCCTGTATGCTGTGCTGGTCAGCGCCCTTGTGTTGATGGCCATGGTCAAGAGAAAGGATTTCTGA
Cluster a Block V
  (SEQ ID NO. 018)
MGTRLFFYVALCLLWAGHRDAGITQSPRYKITETGRQVTLMCHQTWSHSYMFWYRQDLGHGLRLIYYSAAADITDKGEVPDG
YVVSRSKTENFPLTLESATRSQTSVYF
Seq-ID a.1b
(SEQ ID NO. 001)
CASSNDGMNTEAFF
Seq-ID a.2b
(SEQ ID NO. 002)
CASSADGMNTEAFF
Seq-ID a.3b
(SEQ ID NO. 003)
CASSEDGMNTEAFF
Seq-ID a.4b
(SEQ ID NO. 004)
CASSDDGMNTEAFF
Seq-ID a.5b
  (SEQ ID NO. 005)
CASSTDGMNTEAFF
Seq-ID a.6b
  (SEQ ID NO. 006)
CASSEDGMNTEAFF
Seq-ID a.7b
(SEQ ID NO. 432)
CASSGDGMNTEAFF
Seq-ID a.8b
(SEQ ID NO. 5)
CASSTDGMNTEAFF
Seq-ID a.9b
(SEQ ID NO. 433)
CASSESGENTEAFF
Seq-ID a.10b
  (SEQ ID NO. 433)
CASSESGENTEAFF
Seq-ID a.11b
(SEQ ID NO. 434)
CASSP_GENTEAFF
Seq-ID a.12b
(SEQ ID NO. 435)
CASSPRGENTEAFF
“_” indicates a skipped amino acid
Cluster a Block J/C
  (SEQ ID NO. 019)
GQGTRLTVVEDLNKVFPPEVAVFEPSEAEISHTQKATLVCLATGFFPDHVELSWWVNGKEVHSGVSTDPQPLKEQPALNDSRY
CLSSRLRVSATFWQNPRNHFRCQVQFYGLSENDEWTQDRAKPVTQIVSAEAWGRADCGFTSVSYQQGVLSATILYEILLGKATL
YAVLVSALVLMAMVKRKDF*
Alpha-chain, TRA: TRAV21*01, TRAJ33*01, TRAC*01
Cluster a Block V
  (SEQ ID NO. 020)
ATGGAGACCCTCTTGGGCCTGCTTATCCTTTGGCTGCAGCTGCAATGGGTGAGCAGCAAACAGGAGGTGACGCAGATTCC
TGCAGCTCTGAGTGTCCCAGAAGGAGAAAACTTGGTTCTCAACTGCAGTTTCACTGATAGCGCTATTTACAACCTCCAGTG
GTTTAGGCAGGACCCTGGGAAAGGTCTCACATCTCTGTTGCTTATTCAGTCAAGTCAGAGAGAGCAAACAAGTGGAAGAC
TTAATGCCTCGCTGGATAAATCATCAGGACGTAGTACTTTATACATTGCAGCTTCTCAGCCTGGTGACTCAGCCACCTACCTC
Seq-ID a.1a
(SEQ ID NO. 021)
TGTGCTGTGCTCATGGATAGCAACTATCAGTTAATCTGG
Seq-ID a.2a
(SEQ ID NO. 022)
TGTGCTGTCCTCATGGATAGCAACTATCAGTTAATCTGG
Seq-ID a.3a
(SEQ ID NO. 023)
TGTGCGGCCTTAATGGATAGCAACTATCAGTTAATCTGG
Seq-ID a.7a
  (SEQ ID NO. 444)
TGTGCTGTCCTGATGGATAGCAACTATCAGTTAATCTGG
Seq-ID a.8a
(SEQ ID NO. 445)
TGTGCTGTACTCATGGATAGCAACTATCAGTTAATCTGG
Seq-ID a.9a
(SEQ ID NO. 446)
TGTGCTCCATTGGATAGCAACTATCAGTTAATCTGG
Seq-ID a.10a
  (SEQ ID NO. 447)
TGTGCTGCCCAGGATAGCAACTATCAGTTAATCTGG
Seq-ID a.11a
(SEQ ID NO. 448)
TGTGCTGCCATGGATAGCAACTATCAGTTAATCTGG
Seq-ID a.12a
  (SEQ ID NO. 449)
TGTGCTGCTCTGGATAGCAACTATCAGTTAATCTGG
Cluster a Block J/C
  (SEQ ID NO. 024)
GGCGCTGGGACCAAGCTAATTATAAAGCCAGATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA
Cluster a Block V
  (SEQ ID NO. 025)
METLLGLLILWLQLQWVSSKQEVTQIPAALSVPEGENLVLNCSFTDSAIYNLQWFRQDPGKGLTSLLLIQSSQREQTSGRLN
ASLDKSSGRSTLYIAASQPGDSATYL
Seq-ID a.1a
(SEQ ID NO. 007)
CAVLMDSNYQLIW
Seq-ID a.2a
(SEQ ID NO. 008)
CAVLMDSNYQLIW
Seq-ID a.3a
(SEQ ID NO. 009)
CAALMDSNYQLIW
Seq-ID a.7a
  (SEQ ID NO. 007)
CAVLMDSNYQLIW
Seq-ID a.8a
(SEQ ID NO. 007)
CAVLMDSNYQLIW
Seq-ID a.9a
(SEQ ID NO. 436)
CAPL_DSNYQLIW
Seq-ID a.10a
  (SEQ ID NO. 437)
CAA_QDSNYQLIW
Seq-ID a.11a
  (SEQ ID NO. 438)
CAA_MDSNYQLIW
Seq-ID a.12a
  (SEQ ID NO. 439)
CAAL_DSNYQLIW
“_” indicates a skipped amino acid.
Cluster 2 Block J/C
  (SEQ ID NO. 02)
GAGTKLIIKPDIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGFNLLMTLRLWSS*
```

TABLE 2
Cluster ID b:

| | | CDR3 beta: | CDR3 alpha: | | |
|---|---|---|---|---|---|
| SEQ-ID | Pat-ID | Amino acid seq. | Amino acid seq. | HLA_B_I | HLA_C_I |
| b.1 | P62 | CASSLGPNYEQYV | CAASSNQGGKLIF | B*08:01 | C*07:01 |
| | | (SEQ ID NO. 027) | (SEQ ID NO. 031) | | |
| b.2 | P62 | CASSSGPNYEQYF | | B*08:01 | C*07:01 |
| | | (SEQ ID NO. 028) | | | |
| b.3 | P75 | CASSSQGPYEQYF | | B*08:01 | C*07:01 |
| | | (SEQ ID NO. 029) | | | |
| b.4 | P75 | | CAAFYNQGGKLIF | B*08:01 | C*07:01 |
| | | | (SEQ ID NO. 032) | | |
| b.5 | P62 | CASSIGPNYEQYV | | B*08:01 | C*07:01 |
| | | (SEQ ID NO. 030) | | | |

### Cluster ID b:

Cluster b is associated with HLA-B*08:01; HLA-C*07:01
Beta chain, TRB: TRBV7-6, TRBJ2-7, TRBC1*01
Cluster b Block V
ATGGGCACCAGTCTCCTATGCTGGGTGGTCCTGGGTTTCCTAGGGACAGATCACACAGGTGCTGGAGTCTCCCAGTCTCCC
AGGTACAAAGTCACAAAGAGGGGACAGGATGTAGCTCTCAGGTGTGATCCAATTTCGGGTCATGTATCCCTTTATTGGTAC
CGACAGGCCCTGGGGCAGGGCCCAGAGTTTCTGACTTACTTCAATTATGAAGCCCAACAAGACAAATCAGGGCTGCCCAA
TGATCGGTTCTCTGCAGAGAGGCCTGAGGGATCCATCTCCACTCTGACGATCCAGCGCACAGAGCAGCGGGACTCGGCCA
TGTATCGC (SEQ ID NO. 033)
Seq-ID b.1b TGTGCCAGCAGCTTAGGCCCGAATTACGAGCAGTACGTC (SEQ ID NO. 034)
Seq-ID b.2b TGTGCCAGCAGCTCGGGACCAAACTACGAGCAGTACTTC (SEQ ID NO. 035)
Seq-ID b.3b TGTGCCAGCAGCTCCCAAGGGCCCTACGAGCAGTACTTC (SEQ ID NO. 036)
Seq-ID b.4b TGTGCCAGCAGCTCCCAAGGGCCCTACGAGCAGTACTTC (SEQ ID NO. 037)
Seq-ID b.5b TGTGCCAGCAGCATAGGCCCAAACTACGAGCAGTACGTC (SEQ ID NO. 038)
Cluster b Block J/C
GGGCCGGGCACCAGGCTCACGGTCACAGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 039)
Cluster b Block V
MGTSLLCWVVLGFLGTDHTGAGVSQSPRYKVTKRGQDVALRCDPISGHVSLYWYRQALGQGPEFLTYFNYEAQQDKSGLPND
RFSAERPEGSISTLTIQRTEQRDSAMYR (SEQ ID NO. 040)
Seq-ID b.1b CASSLGPNYEQYV (SEQ ID NO. 027)
Seq-ID b.2b CASSSGPNYEQYF (SEQ ID NO. 028)
Seq-ID b.3b CASSSQGPYEQYF (SEQ ID NO. 029)
Seq-ID b.5b CASSIGPNYEQYV (SEQ ID NO. 030)
Cluster b Block J/C
GPGTRLTVTEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 041)
Alpha-chain, TRA: TRAV13-1, TRAJ23, TRAC*01
Cluster b Block V
ATGACATCCATTCGAGCTGTATTTATATTCCTGTGGCTGCAGCTGGACTTGGTGAATGGAGAGAATGTGGAGCAGCATCCT
TCAACCCTGAGTGTCCAGGAGGGAGACAGCGCTGTTATCAAGTGTACTTATTCAGACAGTGCCTCAAACTACTTCCCTTGG
TATAAGCAAGAACTTGGAAAAGGACCTCAGCTTATTATAGACATTCGTTCAAATGTGGGCGAAAAGAAAGACCAACGAAT
TGCTGTTACATTGAACAAGACAGCCAAACATTTCTCCCTGCACATCACAGAGACCCAACCTGAAGACTCGGCTGTCTACTTC
(SEQ ID NO. 042)
Seq-ID b.1a TGTGCAGCAAGTAGTAACCAGGGAGGAAAGCTTATCTTC (SEQ ID NO. 043)
Seq-ID b.4a TGTGCAGCCTTTTATAACCAGGGAGGAAAGCTTATCTTC (SEQ ID NO. 044)
Cluster b Block J/C
GGACAGGGAACGGAGTTATCTGTGAAACCCAATATCCAGAACCCTGACCCTGCCGTGTACCAGCTGAGAGACTCTAAATC
CAGTGACAAGTCTGTCTGCCTATTCACCGATTTTGATTCTCAAACAAATGTGTCACAAAGTAAGGATTCTGATGTGTATATC
ACAGACAAAACTGTGCTAGACATGAGGTCTATGGACTTCAAGAGCAACAGTGCTGTGGCCTGGAGCAACAAATCTGACTT
TGCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 045)
Cluster b Block V
MTSIRAVFIFLWLQLDLVNGENVEQHPSTLSVQEGDSAVIKCTYSDSASNYFPWYKQELGKGPQLIIDIRSNVGEKKDQRIAVTL
NKTAKHFSLHITETQPEDSAVYF (SEQ ID NO. 046)
Seq-ID b.1a CAASSNQGGKLIF (SEQ ID NO. 031)
Seq-ID b.4a CAAFYNQGGKLIF (SEQ ID NO. 032)
Cluster b Block J/C
GQGTELSVKPNIQNPDPAVYQLRDSKSSDKSVCLFTDFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGFRILLLKVAGENLLMTLRLWSS* (SEQ ID NO. 047)

TABLE 3
Cluster ID c:

| | | CDR3 beta: | CDR3 alpha: | |
|---|---|---|---|---|
| SEQ-ID | Pat-ID | Amino acid seq. | Amino acid seq. | HLA_A_I |
| c.1 | P87 | CASSLEGQASSYEQYF | CAGSGAGSYQLTF | A*02:01 |
| | | (SEQ ID NO. 048) | (SEQ ID NO. 052) | |
| c.2 | P91 | CASSLEGQAASYEQYF | CAGPGAGSYQLTF | A*02:01 |
| | | (SEQ ID NO. 049) | (SEQ ID NO. 053) | |
| c.3 | P87 | CASSLEGQASSYEQYF | | A*02:01 |
| | | (SEQ ID NO. 050) | | |
| c.4 | P86 | CASSLEGQASSYEQYF | CAGSGAGSYQLTF | A*02:01 |
| | | (SEQ ID NO. 051) | (SEQ ID NO. 054) | |

### Cluster ID c:

Cluster c is associated with HLA-A*02:01
Beta chain, TRB: TRBV5-1, TRBJ2-7, TRBC2*01
Cluster c Block V
ATGGGCTCCAGGCTGCTCTGTTGGGTGCTGCTTTGTCTCCTGGGAGCAGGCCCAGTAAAGGCTGGAGTCACTCAAACTCC
AAGATATCTGATCAAAACGAGAGGACAGCAAGTGACACTGAGCTGCTCCCCTATCTCTGGGCATAGGAGTGTATCCTGGT
ACCAACAGACCCCAGGACAGGGCCTTCAGTTCCTCTTTGAATACTTCAGTGAGACACAGAGAAACAAAGGAAACTTCCCTG
GTCGATTCTCAGGGCGCCAGTTCTCTAACTCTCGCTCTGAGATGAATGTGAGCACCTTGGAGCTGGGGGACTCGGCCCTTT
ATCTT (SEQ ID NO. 055)
Seq-ID c.1b TGCGCCAGCAGCTTGGAGGGACAGGCGAGCTCCTACGAGCAGTACTTC (SEQ ID NO. 056)
Seq-ID c.2b TGCGCCAGCAGCTTGGAAGGACAGGCAGCCTCCTACGAGCAGTACTTC (SEQ ID NO. 057)
Seq-ID c.3b TGCGCCAGCAGCTTGGAGGGACAGGCGAGCTCCTACGAGCAGTACTTC (SEQ ID NO. 058)
Seq-ID c.4b TGCGCCAGCAGCTTGGAGGGGCAGGCTAGCTCCTACGAGCAGTACTTC (SEQ ID NO. 059)
Cluster c Block J/C
GGGCCGGGCACCAGGCTCACGGTCACAGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 060)
Cluster c Block V
MGSRLLCWVLLCLLGAGPVKAGVTQTPRYLIKTRGQQVTLSCSPISGHRSVSWYQQTPGQGLQFLFEYFSETQRNKGNFPGRFS
GRQFSNSRSEMNVSTLELGDSALYL (SEQ ID NO. 061)
Seq-ID c.1b CASSLEGQASSYEQYF (SEQ ID NO. 048)
Seq-ID c.2b CASSLEGQAASYEQYF (SEQ ID NO. 049)
Seq-ID c.3b CASSLEGQASSYEQYF (SEQ ID NO. 050)
Seq-ID c.4b CASSLEGQASSYEQYF (SEQ ID NO. 051)
Cluster c Block J/C
GPGTRLTVTEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 062)
Alpha-chain, TRA: TRAV25, TRAJ28, TRAC*01
Cluster c Block V
ATGCTACTCATCACATCAATGTTGGTCTTATGGATGCAATTGTCACAGGTGAATGGACAACAGGTAATGCAAATTCCTCAG
TACCAGCATGTACAAGAAGGAGAGGACTTCACCACGTACTGCAATTCCTCAACTACTTTAAGCAATATACAGTGGTATAAG
CAAAGGCCTGGTGGACATCCCGTTTTTTTGATACAGTTAGTGAAGAGTGGAGAAGTGAAGAAGCAGAAAAGACTGACATT
TCAGTTTGGAGAAGCAAAAAAGAACAGCTCCCTGCACATCACAGCCACCCAGACTACAGATGTAGGAACCTACTTC (SEQ
ID NO. 063)
Seq-ID c.1a TGTGCAGGATCTGGGGCTGGGAGTTACCAACTCACTTTC (SEQ ID NO. 064)
Seq-ID c.2a TGTGCAGGCCCTGGGGCTGGGAGTTACCAACTCACTTTC (SEQ ID NO. 065)
Seq-ID c.4a TGTGCGGGGTCGGGGGCTGGGAGTTACCAACTCACTTTC (SEQ ID NO. 066)
Cluster c Block J/C
GGGAAGGGGACCAAACTCTCGGTCATACCAAATATCCAGAACCCTGACCCTGCCGTGTACCAGCTGAGAGACTCTAAATC
CAGTGACAAGTCTGTCTGCCTATTCACCGATTTTGATTCTCAAACAAATGTGTCACAAAGTAAGGATTCTGATGTGTATATC
ACAGACAAAACTGTGCTAGACATGAGGTCTATGGACTTCAAGAGCAACAGTGCTGTGGCCTGGAGCAACAAATCTGACTT
TGCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 067)
Cluster c Block V
MLLITSMLVLWMQLSQVNGQQVMQIPQYQHVQEGEDFTTYCNSSTTLSNIQWYKQRPGGHPVFLIQLVKSGEVKKQKRLTF
QFGEAKKNSSLHITATQTTDVGTYF (SEQ ID NO. 068)
Seq-ID c.1a CAGSGAGSYQLTF (SEQ ID NO. 052)
Seq-ID c.2a CAGPGAGSYQLTF (SEQ ID NO. 053)
Seq-ID c.4a CAGSGAGSYQLTF (SEQ ID NO. 054)
Cluster c Block J/C
GKGTKLSVIPNIQNPDPAVYQLRDSKSSDKSVCLFTDFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGFRILLLKVAGFNLLMTLRLWSS* (SEQ ID NO. 069)

TABLE 4
Cluster ID d:

| | | CDR3 beta: | CDR3 alpha: | |
|---|---|---|---|---|
| SEQ-ID | Pat-ID | Amino acid seq. | Amino acid seq. | |
| d.1 | P86 | CSVGAGGTNEKLFF | CAESISAGKIIF | HLA_A_I |
| | | (SEQ ID NO. 070) | (SEQ ID NO. 073) | A*02:01 |
| d.2 | P93 | CSVGAGGTNEKLFF | | |
| | | (SEQ ID NO. 071) | | |
| d.3 | P87 | CSVGTVATNEKLFF | | |
| | | (SEQ ID NO. 072) | | |

### Cluster d Block V

Cluster d is associated with HLA-A*02:01
Beta chain, TRB: TRBV29-1, TRBJ1-4, TRBC1*01
Cluster d Block V
ATGCTGAGTCTTCTGCTCCTTCTCCTGGGACTAGGCTCTGTGTTCAGTGCTGTCATCTCTCAAAAGCCAAGCAGGGATATCT
GTCAACGTGGAACCTCCCTGACGATCCAGTGTCAAGTCGATAGCCAAGTCACCATGATGTTCTGGTACCGTCAGCAACCTG
GACAGAGCCTGACACTGATCGCAACTGCAAATCAGGGCTCTGAGGCCACATATGAGAGTGGATTTGTCATTGACAAGTTT
CCCATCAGCCGCCCAAACCTAACATTCTCAACTCTGACTGTGAGCAACATGAGCCCTGAAGACAGCAGCATATATCTC
(SEQ ID NO. 074)
Seq-ID d.1b TGCAGCGTTGGAGCTGGAGGAACTAATGAAAAACTGTTTTTT (SEQ ID NO. 075)
Seq-ID d.2b TGCAGCGTTGGGGCAGGGGGCACTAATGAAAAACTGTTTTTT (SEQ ID NO. 076)
Seq-ID d.3b TGCAGCGTGGGGACGGTGGCAACTAATGAAAAACTGTTTTTT (SEQ ID NO. 077)
Cluster d Block J/C
GGCAGTGGAACCCAGCTCTCTGTCTTGGAGGACCTGAACAAGGTGTTCCCACCCGAGGTCGCTGTGTTTGAGCCATCAGA
AGCAGAGATCTCCCACACCCAAAAGGCCACACTGGTGTGCCTGGCCACAGGCTTCTTCCCTGACCACGTGGAGCTGAGCT
GGTGGGTGAATGGGAAGGAGGTGCACAGTGGGGTCAGCACGGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCCGTCACCCAGATCGTCAGCGCCGAG
GCCTGGGGTAGAGCAGACTGTGGCTTTACCTCGGTGTCCTACCAGCAAGGGGTCCTGTCTGCCACCATCCTCTATGAGATC
CTGCTAGGGAAGGCCACCCTGTATGCTGTGCTGGTCAGCGCCCTTGTGTTGATGGCCATGGTCAAGAGAAAGGATTTCTG
A (SEQ ID NO. 078)
Cluster d Block V
MLSLLLLLLGLGSVFSAVISQKPSRDICQRGTSLTIQCQVDSQVTMMFWYRQQPGQSLTLIATANQGSEATYESGFVIDKFPISRP
NLTFSTLTVSNMSPEDSSIYL (SEQ ID NO. 079)
Seq-ID d.1b CSVGAGGTNEKLFF (SEQ ID NO. 070)
Seq-ID d.2b CSVGAGGTNEKLFF (SEQ ID NO. 071)
Seq-ID d.3b CSVGTVATNEKLFF (SEQ ID NO. 072)
Cluster d Block J/C
GSGTQLSVLEDLNKVFPPEVAVFEPSEAEISHTQKATLVCLATGFFPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSVSYQQGVLSATILYEILLGKATLY
AVLVSALVLMAMVKRKDF* (SEQ ID NO. 080)
Alpha-chain, TRA: TRAV5, TRAJ37/34/30, TRAC*01
Cluster d Block V
ATGAAGACATTTGCTGGATTTTCGTTCCTGTTTTTGTGGCTGCAGCTGGACTGTATGAGTAGAGGAGAGGATGTGGAGCA
GAGTCTTTTCCTGAGTGTCCGAGAGGGAGACAGCTCCGTTATAAACTGCACTTACACAGACAGCTCCTCCACCTACTTATAC
TGGTATAAGCAAGAACCTGGAGCAGGTCTCCAGTTGCTGACGTATATTTTTTCAAATATGGACATGAAACAAGACCAAAGA
CTCACTGTTCTATTGAATAAAAAGGATAAACATCTGTCTCTGCGCATTGCAGACACCCAGACTGGGGACTCAGCTATCTACT
TC (SEQ ID NO. 081)
Seq-ID d.1a TGTGCAGAGAGTATAAGCGCGGGCAAGATCATCTTT (SEQ ID NO. 082)
Cluster d Block J/C
GGAAAAGGGACACGACTTCATATTCTCCCCAATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 083)
Cluster d Block V
MKTFAGFSFLFLWLQLDCMSRGEDVEQSLFLSVREGDSSVINCTYTDSSSTYLYWYKQEPGAGLQLLTYIFSNMDMKQDQRLT
VLLNKKDKHLSLRIADTQTGDSAIYF (SEQ ID NO. 084)
Seq-ID d.1a CAESISAGKIIF (SEQ ID NO. 073)
Cluster d Block J/C
GKGTRLHILPNIQNPDPAVYQLRDSKSSDKSVCLFTDFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGFRILLLKVAGFNLLMTLRLWSS* (SEQ ID NO. 085)

TABLE 5
Cluster ID e:

| | | CDR3 beta: | CDR3 alpha: | |
|---|---|---|---|---|
| SEQ-ID | Pat-ID | Amino acid seq. | Amino acid seq. | HLA_B_I |
| e.1 | P91 | CASSVTSGAYNEQFF | CVVSAGREYGNKLVF | B*15:01 |
| | | (SEQ ID NO. 086) | (SEQ ID NO. 100) | |
| e.2 | P82 | CASQGTSGAYNEQFF | | B*15:01 |
| | | (SEQ ID NO. 087) | | |
| e.3 | P82 | CASSITSGNYNEQFF | | B*15:01 |
| | | (SEQ ID NO. 088) | | |
| e.4 | P82 | CASSMTSGSYNEQFF | | B*15:01 |
| | | (SEQ ID NO. 089) | | |
| e.5 | P82 | CASSPTSGSYNEQFF | | B*15:01 |
| | | (SEQ ID NO. 090) | | |
| e.6 | P82 | CASSRTSGGYNEQFF | | B*15:01 |
| | | (SEQ ID NO. 091) | | |
| e.7 | P82 | CASSVTSGAYNEQFF | | B*15:01 |
| | | (SEQ ID NO. 092) | | |
| e.8 | P84 | CASSATSGNYNEQFF | | B*15:01 |
| | | (SEQ ID NO. 093) | | |
| e.9 | P84 | CASSATSGSYNEQFF | | B*15:01 |
| | | (SEQ ID NO. 094) | | |
| e.10 | P84 | CASSITSGDYNEQFF | | B*15:01 |
| | | (SEQ ID NO. 095) | | |
| e.11 | P84 | CASSKTSGDYNEQFF | | B*15:01 |
| | | (SEQ ID NO. 096) | | |
| e.12 | P84 | CASSPTSGQYNEQFF | | B*15:01 |
| | | (SEQ ID NO. 097) | | |
| e.13 | P91 | CASSVTSGSYNEQFF | | B*15:01 |
| | | (SEQ ID NO. 098) | | |
| e.14 | P91 | CASSLTSGDYNEQFF | | B*15:01 |
| | | (SEQ ID NO. 099) | | |
| e.15 | P98 | CASSISSGSYNEQFF | CVVTPGREYGNKLVF | B*15:01 |
| | | (SEQ ID NO. 450) | (SEQ ID NO. 455) | |
| e.16 | PANC09 | CASSISSGSYNEQFF | CVVSSGREYGNKLVF | |
| | | (SEQ ID NO. 451) | (SEQ ID NO. 456) | |
| e.17 | P98 | CASSITSGSYDEQFF | CVVSTGREYGNKLVF | |
| | | (SEQ ID NO. 452) | (SEQ ID NO. 457) | |
| e.18 | P82 | CASSITSGSYNEQFF | CVVSAGREYGNKLVF | |
| | | (SEQ ID NO. 453) | (SEQ ID NO. 458) | |
| e.19 | P98 | CASTMTSGAYNEQFF | CVVSAGREYGNKLVF | |
| | | (SEQ ID NO. 454) | (SEQ ID NO. 459) | |

### Cluster ID e:

Cluster e is associated with HLA-B*15:01
Beta chain, TRB: TRBV19, TRBJ12-1, TRBC2*01
Cluster e Block V
ATGAGCAACCAGGTGCTCTGCTGTGTGGTCCTTTGTTTCCTGGGAGCAAACACCGTGGATGGTGGAATCACTCAGTCCCCA
AAGTACCTGTTCAGAAAGGAAGGACAGAATGTGACCCTGAGTTGTGAACAGAATTTGAACCACGATGCCATGTACTGGTA
CCGACAGGACCCAGGGCAAGGGCTGAGATTGATCTACTACTCACAGATAGTAAATGACTTTCAGAAAGGAGATATAGCTG
AAGGGTACAGCGTCTCTCGGGAGAAGAAGGAATCCTTTCCTCTCACTGTGACATCGGCCCAAAAGAACCCGACAGCTTTCT
ATCTC (SEQ ID NO. 0101)
Seq-ID e.1b TGTGCCAGTAGTGTCACTAGCGGGGCTTACAATGAGCAGTTCTTC (SEQ ID NO. 102)
Seq-ID e.2b TGTGCCAGTCAGGGGACTAGCGGGGCCTACAATGAGCAGTTCTTC (SEQ ID NO. 103)
Seq-ID e.3b TGTGCCAGTAGTATAACTAGCGGGAACTACAATGAGCAGTTCTTC (SEQ ID NO. 104)
Seq-ID e.4b TGTGCCAGTAGTATGACTAGCGGTTCCTACAATGAGCAGTTCTTC (SEQ ID NO. 105)
Seq-ID e.5b TGTGCCAGTAGCCCAACTAGCGGATCCTACAATGAGCAGTTCTTC (SEQ ID NO. 106)
Seq-ID e.6b TGTGCCAGTAGTCGGACTAGCGGGGGCTACAATGAGCAGTTCTTC (SEQ ID NO. 107)
Seq-ID e.7b TGTGCCAGTAGTGTAACTAGCGGGGCCTACAATGAGCAGTTCTTC (SEQ ID NO. 108)
Seq-ID e.8b TGTGCCAGTAGTGCGACTAGCGGGAACTACAATGAGCAGTTCTTC (SEQ ID NO. 109)
Seq-ID e.9b TGTGCCAGTAGTGCCACTAGCGGGAGCTACAATGAGCAGTTCTTC (SEQ ID NO. 110)
Seq-ID e.10b TGTGCCAGTAGTATAACTAGCGGAGATTACAATGAGCAGTTCTTC (SEQ ID NO. 111)
Seq-ID e.11b TGTGCCAGTAGTAAAACTAGCGGAGACTACAATGAGCAGTTCTTC (SEQ ID NO. 112)
Seq-ID e.12b TGTGCCAGTAGTCCCACTAGCGGCCAGTACAATGAGCAGTTCTTC (SEQ ID NO. 113)
Seq-ID e.13b TGTGCCAGTAGCGTGACTAGCGGCTCCTACAATGAGCAGTTCTTC (SEQ ID NO. 114)
Seq-ID e.14b TGTGCCAGTAGTTTGACTAGCGGGGACTACAATGAGCAGTTCTTC (SEQ ID NO. 115)
Seq-ID e.15b TGTGCCAGTAGTATTTCTAGCGGATCCTACAATGAGCAGTTCTTC (SEQ ID NO. 460)
Seq-ID e.16b TGTGCCAGTAGTATAAGTAGCGGGAGCTACAATGAGCAGTTCTTC (SEQ ID NO. 461)
Seq-ID e.17b TGTGCCAGTAGTATAACTAGCGGGAGTTACGATGAGCAGTTCTTC (SEQ ID NO. 462)
Seq-ID e.18b TGTGCCAGTAGTATAACTAGCGGTTCCTACAATGAGCAGTTCTTC (SEQ ID NO. 463)
Seq-ID e.19b TGTGCCAGTACCATGACTAGCGGGGCCTACAATGAGCAGTTCTTC (SEQ ID NO. 464)
Cluster e Block J/C
GGGCCAGGGACACGGCTCACCGTGCTAGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 116)
Cluster e Block V
MSNQVLCCVVLCFLGANTVDGGITQSPKYLFRKEGQNVTLSCEQNLNHDAMYWYRQDPGQGLRLIYYSQIVNDFQKGDIAEG
YSVSREKKESFPLTVTSAQKNPTAFYL (SEQ ID NO. 117)
Seq-ID e.1b CASSVTSGAYNEQFF (SEQ ID NO. 086)
Seq-ID e.2b CASQGTSGAYNEQFF (SEQ ID NO. 087)
Seq-ID e.3b CASSITSGNYNEQFF (SEQ ID NO. 088)
Seq-ID e.4b CASSMTSGSYNEQFF (SEQ ID NO. 089)
Seq-ID e.5b CASSPTSGSYNEQFF (SEQ ID NO. 090)
Seq-ID e.6b CASSRTSGGYNEQFF (SEQ ID NO. 091)
Seq-ID e.7b CASSVTSGAYNEQFF (SEQ ID NO. 092)
Seq-ID e.8b CASSATSGNYNEQFF (SEQ ID NO. 093)
Seq-ID e.9b CASSATSGSYNEQFF (SEQ ID NO. 094)
Seq-ID e.10b CASSITSGDYNEQFF (SEQ ID NO. 095)
Seq-ID e.11b CASSKTSGDYNEQFF (SEQ ID NO. 096)
Seq-ID e.12b CASSPTSGQYNEOFF (SEQ ID NO. 097)
Seq-ID e.13b CASSVTSGSYNEQFF (SEQ ID NO. 098)
Seq-ID e.14b CASSLTSGDYNEQFF (SEQ ID NO. 099)
Seq-ID e.15b CASSISSGSYNEQFF (SEQ ID NO. 450)
Seq-ID e.16b CASSISSGSYNEQFF (SEQ ID NO. 451)
Seq-ID e.17b CASSITSGSYDEQFF (SEQ ID NO. 452)
Seq-ID e.18b CASSITSGSYNEQFF (SEQ ID NO. 453)
Seq-ID e.19b CASTMTSGAYNEQFF ( SEQ ID NO. 454)
Cluster e Block J/C
GPGTRLTVLEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 118)
Alpha-chain, TRA: TRAV10, TRAJ47, TRAC*01
Cluster e Block V
ATGAAAAAGCATCTGACGACCTTCTTGGTGATTTTGTGGCTTTATTTTTATAGGGGGAATGGCAAAAACCAAGTGGAGCA
GAGTCCTCAGTCCCTGATCATCCTGGAGGGAAAGAACTGCACTCTTCAATGCAATTATACAGTGAGCCCCTTCAGCAACTT
AAGGTGGTATAAGCAAGATACTGGGAGAGGTCCTGTTTCCCTGACAATCATGACTTTCAGTGAGAACACAAAGTCGAACG
GAAGATATACAGCAACTCTGGATGCAGACACAAAGCAAAGCTCTCTGCACATCACAGCCTCCCAGCTCAGCGATTCAGCCT
CCTACATC (SEQ ID NO. 119)
Seq-ID e.1a TGTGTGGTGAGCGCCGGGAGGGAATATGGAAACAAACTGGTCTTT (SEQ ID NO. 120)
Seq-ID e.15a-TGTGTGGTGACCCCCGGTAGGGAATATGGAAACAAACTGGTCTTT (SEQ ID NO. 465)
Seq-ID e.16a-TGTGTGGTGAGCTCCGGCAGGGAATATGGAAACAAACTGGTCTTT (SEQ ID NO. 466)
Seq-ID e.17a-TGTGTGGTGAGCACGGGGAGGGAATATGGAAACAAACTGGTCTTT (SEQ ID NO. 467)
Seq-ID e.18a-TGTGTGGTGAGCGCGGGTCGGGAATATGGAAACAAACTGGTCTTT (SEQ ID NO. 468)
Seq-ID e.19a-TGTGTGGTGAGCGCGGGTAGGGAATATGGAAACAAACTGGTCTTT (SEQ ID NO. 469)
Cluster e Block J/C
GGCGCAGGAACCATTCTGAGAGTCAAGTCCTATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 121)
Cluster e Block V
MKKHLTTFLVILWLYFYRGNGKNQVEQSPQSLIILEGKNCTLQCNYTVSPFSNLRWYKQDTGRGPVSLTIMTFSENTKSNGRYTA
TLDADTKQSSLHITASQLSDSASYI (SEQ ID NO. 122)
Seq-ID e.1a CVVSAGREYGNKLVF (SEQ ID NO. 100)
Seq-ID e.15a CVVTPGREYGNKLVF (SEQ ID NO. 455)
Seq-ID e.16a CVVSSGREYGNKLVF (SEQ ID NO. 456)
Seq-ID e.17a CVVSTGREYGNKLVF (SEQ ID NO. 457)
Seq-ID e.18a CVVSAGREYGNKLVF (SEQ ID NO. 458)
Seq-ID e.19a CVVSAGREYGNKLVF (SEQ ID NO. 459)
Cluster e Block J/C
GAGTILRVKSYIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGENLLMTLRLWSS* (SEQ ID NO. 123)

TABLE 6
Cluster ID f:

| | | CDR3 beta: | CDR3 alpha: | |
|---|---|---|---|---|
| SEQ-ID | Pat-ID | Amino acid seq. | Amino acid seq. | HLA A_I |
| f.1 | P91 | CASSTTSGAYNEQFF | CAVNRGRDAGGTSYGKLTF | A*02:01 |
| | | (SEQ ID NO. 124) | (SEQ ID NO. 131) | |
| f.2 | P82 | CASSTTSGDYNEQFF | CAVKLGRDAGGTSYGKLTF | A*02:01 |
| | | (SEQ ID NO. 125) | (SEQ ID NO. 132) | |
| f.3 | P82 | CASSVTSGAYNEQFF | | A*02:01 |
| | | (SEQ ID NO. 126) | | |
| f.4 | P82 | CASSPTSGQYNEQFF | | A*02:01 |
| | | (SEQ ID NO. 127) | | |
| f.5 | P82 | CASSQTSGGYNEQFF | | A*02:01 |
| | | (SEQ ID NO. 128) | | |
| f.6 | P82 | CASSVTSGAYNEQFF | | A*02:01 |
| | | (SEQ ID NO. 129) | | |
| f.7 | P91 | CASSLTSGGYNEQFF | | A*02:01 |
| | | (SEQ ID NO. 130) | | |
| f.8 | P82 | CASSITSGDYNEQFF | CAVRRGRDGGTSYGKLTF | A*02:01 |
| | | (SEQ ID NO. 470) | (SEQ ID NO. 472) | |
| f.9 | P91 | CASSMTSGSYNEQFF | CAVRASRDAGGTSYGKLTF | A*02:01 |
| | | (SEQ ID NO. 471) | (SEQ ID NO. 473) | |

Cluster ID f:
Cluster f is associated with HLA-A*02:01
Beta chain, TRB: TRBV19, TRBJ2-1, TRBC2*01
Cluster f Block V
ATGAGCAACCAGGTGCTCTGCTGTGTGGTCCTTTGTTTCCTGGGAGCAAACACCGTGGATGGTGGAATCACTCAGTCCCCA
AAGTACCTGTTCAGAAAGGAAGGACAGAATGTGACCCTGAGTTGTGAACAGAATTTGAACCACGATGCCATGTACTGGTA
CCGACAGGACCCAGGGCAAGGGCTGAGATTGATCTACTACTCACAGATAGTAAATGACTTTCAGAAAGGAGATATAGCTG
AAGGGTACAGCGTCTCTCGGGAGAAGAAGGAATCCTTTCCTCTCACTGTGACATCGGCCCAAAAGAACCCGACAGCTTTCT
ATCTC (SEQ ID NO. 133)
Seq-ID f.1b TGTGCCAGTAGTACCACCTCCGGGGCCTACAATGAGCAGTTCTTC (SEQ ID NO. 134)
Seq-ID f.2b TGTGCCAGTAGTACGACTAGCGGGGACTACAATGAGCAGTTCTTC (SEQ ID NO. 135)
Seq-ID f.3b TGTGCCAGTAGTGTAACTAGCGGGGCTTACAATGAGCAGTTCTTC (SEQ ID NO. 136)
Seq-ID f.4b TGTGCCAGTAGCCCGACTAGCGGACAATACAATGAGCAGTTCTTC (SEQ ID NO. 137)
Seq-ID f.5b TGTGCCAGTAGCCAAACTAGCGGGGGATACAATGAGCAGTTCTTC (SEQ ID NO. 138)
Seq-ID f.6b TGTGCCAGTAGTGTCACTAGCGGGGCCTACAATGAGCAGTTCTTC (SEQ ID NO. 139)
Seq-ID f.7b TGTGCCAGTAGCCTAACTAGCGGGGGATACAATGAGCAGTTCTTC (SEQ ID NO. 140)
Seq-ID f.8b TGTGCCAGTAGTATTACTTCGGGGGATTACAATGAGCAGTTCTTC (SEQ ID NO. 474)
Seq-ID f.9b TGTGCCAGTAGTATGACTAGCGGGTCCTACAATGAGCAGTTCTTC (SEQ ID NO. 475)
Cluster f Block J/C
GGGCCAGGGACACGGCTCACCGTGCTAGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 141)
Cluster f Block V
MSNQVLCCVVLCFLGANTVDGGITQSPKYLFRKEGQNVTLSCEQNLNHDAMYWYRQDPGQGLRLIYYSQIVNDFQKGDIAEG
YSVSREKKESFPLTVTSAQKNPTAFYL (SEQ ID NO. 142)
Seq-ID f.1b CASSTTSGAYNEQFF (SEQ ID NO. 124)
Seq-ID f.2b CASSTTSGDYNEQFF (SEQ ID NO. 125)
Seq-ID f.3b CASSVTSGAYNEQFF (SEQ ID NO.
Seq-ID f.4b CASSPTSGQYNEQFF (SEQ ID NO. 127)
Seq-ID f.5b CASSQTSGGYNEQFF (SEQ ID NO. 128)
Seq-ID f.6b CASSVTSGAYNEQFF (SEQ ID NO. 129)
Seq-ID f.7b CASSLTSGGYNEQFF (SEQ ID NO. 130)
Seq-ID f.8b CASSITSGDYNEQFF (SEQ ID NO. 470)
Seq-ID f9.b CASSMTSGSYNEQFF (SEQ ID NO. 471)
Cluster f Block J/C
GPGTRLTVLEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 143)
Alpha-chain, TRA: TRAV12-2, TRAJ52, TRAC*01
Cluster f Block V
ATGAAATCCTTGAGAGTTTTACTAGTGATCCTGTGGCTTCAGTTGAGCTGGGTTTGGAGCCAACAGAAGGAGGTGGAGCA
GAATTCTGGACCCCTCAGTGTTCCAGAGGGAGCCATTGCCTCTCTCAACTGCACTTACAGTGACCGAGGTTCCCAGTCCTTC
TTCTGGTACAGACAATATTCTGGGAAAAGCCCTGAGTTGATAATGTTCATATACTCCAATGGTGACAAAGAAGATGGAAG
GTTTACAGCACAGCTCAATAAAGCCAGCCAGTATGTTTCTCTGCTCATCAGAGACTCCCAGCCCAGTGATTCAGCCACCTAC
CTC (SEQ ID NO. 144)
Seq-ID f.1a TGTGCCGTGAATAGGGGAAGGGATGCTGGTGGTACTAGCTATGGAAAGCTGACATTT (SEQ ID
NO. 145)
Seq-ID f.2a TGTGCCGTGAAATTAGGGCGGGATGCTGGTGGTACTAGCTATGGAAAGCTGACATTT (SEQ ID
NO. 146)
Seq-ID f.8a TGTGCCGTGAGGAGGGGTCGAGATGGTGGTACTAGCTATGGAAAGCTGACATTT (SEQ ID NO.
476)
Seq-ID f.9a TGTGCCGTGAGGGCTTCGAGGGATGCTGGTGGTACTAGCTATGGAAAGCTGACATTT (SEQ ID
NO. 477)
Cluster f Block J/C
GGACAAGGGACCATCTTGACTGTCCATCCAAATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 147)
Cluster f Block V
MKSLRVLLVILWLQLSWVWSQQKEVEQNSGPLSVPEGAIASLNCTYSDRGSQSFFWYRQYSGKSPELIMFIYSNGDKEDGRFTA
QLNKASQYVSLLIRDSQPSDSATYL (SEQ ID NO. 148)
Seq-ID f.1a CAVNRGRDAGGTSYGKLTF (SEQ ID NO. 131)
Seq-ID f.2a CAVKLGRDAGGTSYGKLTF (SEQ ID NO. 132)
Seq-ID f.8a CAVRRGRD_GGTSYGKLTF (SEQ ID NO. 472)
Seq-ID f.9a CAVRASRDAGGTSYGKLTF (SEQ ID NO. 473)
Cluster f Block J/C
GQGTILTVHPNIQNPDPAVYQLRDSKSSDKSVCLFTDFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGFRILLLKVAGFNLLMTLRLWSS* (SEQ ID NO. 149)

TABLE 7
Cluster ID g:

| | | CDR3 beta: | CDR3 alpha: | |
|---|---|---|---|---|
| SEQ-ID | Pat-ID | Amino acid seq. | Amino acid seq. | HLA_B_I |
| g.1 | P73 | CASSYDSSYEQYF | CAGPYFGNEKLTF | B*08:01 |
| | | (SEQ ID NO. 150) | (SEQ ID NO. 156) | |
| g.2 | P62 | CASSYDSSYEQYV | | B*08:01 |
| | | (SEQ ID NO. 151) | | |
| g.3 | P73 | CASSWDSSYEQYF | | B*08:01 |
| | | (SEQ ID NO. 152) | | |
| g.4 | P73 | CASSIDSSYEQYF | | B*08:01 |
| | | (SEQ ID NO. 153) | | |
| g.5 | P73 | CASSIDSSYEQYF | | B*08:01 |
| | | (SEQ ID NO. 154) | | |
| g.6 | P73 | CASTVDSSYEQYF | | B*08:01 |
| | | (SEQ ID NO. 155) | | |

### Cluster ID g:

Cluster g is associated with HLA-B*08:01
Beta chain, TRB: TRBV6-2, TRBJ2-7, TRBC2*01
Cluster g Block V
ATGAGCCTCGGGCTCCTGTGCTGTGGGGCCTTTTCTCTCCTGTGGGCAGGTCCAGTGAATGCTGGTGTCACTCAGACCCCA
AAATTCCGGGTCCTGAAGACAGGACAGAGCATGACACTGCTGTGTGCCCAGGATATGAACCATGAATACATGTACTGGTA
TCGACAAGACCCAGGCATGGGGCTGAGGCTGATTCATTACTCAGTTGGTGAGGGTACAACTGCCAAAGGAGAGGTCCCT
GATGGCTACAATGTCTCCAGATTAAAAAAACAGAATTTCCTGCTGGGGTTGGAGTCGGCTGCTCCCTCCCAAACATCTGTG
TACTTC (SEQ ID NO. 157)
Seq-ID g.1b TGTGCCAGCAGTTACGACAGCTCCTACGAGCAGTACTTC (SEQ ID NO. 158)
Seq-ID g.2b TGTGCCAGCAGTTACGACAGCTCCTACGAGCAGTACGTC (SEQ ID NO. 159)
Seq-ID g.3b TGTGCCAGCAGCTGGGACTCCTCCTACGAGCAGTACTTC (SEQ ID NO. 160)
Seq-ID g.4b TGTGCCAGCTCGATAGACAGCTCCTACGAGCAGTACTTC (SEQ ID NO. 161)
Seq-ID g.5b TGTGCCAGCAGTATAGACAGCTCCTACGAGCAGTACTTC (SEQ ID NO. 162)
Seq-ID g.6b TGTGCCAGCACCGTCGACTCGTCCTACGAGCAGTACTTC (SEQ ID NO. 163)
Cluster g Block J/C
GGGCCGGGCACCAGGCTCACGGTCACAGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 164)
Cluster g Block V
MSLGLLCCGAFSLLWAGPVNAGVTQTPKFRVLKTGQSMTLLCAQDMNHEYMYWYRQDPGMGLRLIHYSVGEGTTAKGEVP
DGYNVSRLKKQNFLLGLESAAPSQTSVYF (SEQ ID NO. 165)
Seq-ID g.1b CASSYDSSYEQYF (SEQ ID NO. 150)
Seq-ID g.2b CASSYDSSYEQYV (SEQ ID NO. 151)
Seq-ID g.3b CASSWDSSYEQYF (SEQ ID NO. 152)
Seq-ID g.4b CASSIDSSYEQYF (SEQ ID NO. 153)
Seq-ID g.5b CASSIDSSYEQYF (SEQ ID NO. 154)
Seq-ID g.6b CASTVDSSYEQYF (SEQ ID NO. 155)
Cluster g Block J/C
GPGTRLTVTEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 166)
Alpha-chain, TRA: TRAV35, TRAJ48, TRAC*01
Cluster g Block V
ATGCTCCTTGAACATTTATTAATAATCTTGTGGATGCAGCTGACATGGGTCAGTGGTCAACAGCTGAATCAGAGTCCTCAA
TCTATGTTTATCCAGGAAGGAGAAGATGTCTCCATGAACTGCACTTCTTCAAGCATATTTAACACCTGGCTATGGTACAAGC
AGGACCCTGGGGAAGGTCCTGTCCTCTTGATAGCCTTATATAAGGCTGGTGAATTGACCTCAAATGGAAGACTGACTGCTC
AGTTTGGTATAACCAGAAAGGACAGCTTCCTGAATATCTCAGCATCCATACCTAGTGATGTAGGCATCTACTTC (SEQ ID
NO. 167)
Seq-ID g.1a TGTGCTGGCCCCTACTTTGGAAATGAGAAATTAACCTTT (SEQ ID NO. 168)
Cluster g Block J/C
GGGACTGGAACAAGACTCACCATCATACCCAATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 169)
Cluster g Block V
MLLEHLLIILWMQLTWVSGQQLNQSPQSMFIQEGEDVSMNCTSSSIFNTWLWYKQDPGEGPVLLIALYKAGELTSNGRLTAQF
GITRKDSFLNISASIPSDVGIYF (SEQ ID NO. 170)
Seq-ID g.1a CAGPYFGNEKLTF (SEQ ID NO. 156)
Cluster g Block J/C
GTGTRLTIIPNIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGENLLMTLRLWSS* (SEQ ID NO. 171)

TABLE 8
Cluster ID h:

| | | CDR3 beta: | CDR3 alpha: | |
|---|---|---|---|---|
| SEQ-ID | Pat-ID | Amino acid seq. | Amino acid seq. | HLA_B_I |
| h.1 | P87 | CASSLAGDQPQHF | CAVRYGQNFVF | |
| | | (SEQ ID NO. 172) | (SEQ ID NO. 175) | B*07:02 |
| h.2 | P87 | CASSLEGDRPQHF | CAVRYGQNFVF | B*07:02 |
| | | (SEQ ID NO. 173) | (SEQ ID NO. 176) | |
| h.3 | P14 | CASSLEGDQPQHF | | |
| | | (SEQ ID NO. 174) | | |

### Cluster ID h:

Cluster h is associated with HLA-B*07:02
Beta chain, TRB: TRBV5-1, TRBJ1-5, TRBC1*01
Cluster h Block V
ATGGGCTCCAGGCTGCTCTGTTGGGTGCTGCTTTGTCTCCTGGGAGCAGGCCCAGTAAAGGCTGGAGTCACTCAAACTCC
AAGATATCTGATCAAAACGAGAGGACAGCAAGTGACACTGAGCTGCTCCCCTATCTCTGGGCATAGGAGTGTATCCTGGT
ACCAACAGACCCCAGGACAGGGCCTTCAGTTCCTCTTTGAATACTTCAGTGAGACACAGAGAAACAAAGGAAACTTCCCTG
GTCGATTCTCAGGGCGCCAGTTCTCTAACTCTCGCTCTGAGATGAATGTGAGCACCTTGGAGCTGGGGGACTCGGCCCTTT
ATCTT (SEQ ID NO. 177)
Seq-ID h.1b TGCGCCAGCAGCTTGGCAGGGGATCAGCCCCAGCATTTT (SEQ ID NO. 178)
Seq-ID h.2b TGCGCCAGCAGCTTGGAAGGGGACCGACCCCAGCATTTT (SEQ ID NO. 179)
Seq-ID h.3b TGCGCCAGCAGCTTGGAGGGGGATCAGCCCCAGCATTTT (SEQ ID NO. 180)
Cluster h Block J/C
GGTGATGGGACTCGACTCTCCATCCTAGAGGACCTGAACAAGGTGTTCCCACCCGAGGTCGCTGTGTTTGAGCCATCAGA
AGCAGAGATCTCCCACACCCAAAAGGCCACACTGGTGTGCCTGGCCACAGGCTTCTTCCCTGACCACGTGGAGCTGAGCT
GGTGGGTGAATGGGAAGGAGGTGCACAGTGGGGTCAGCACGGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCCGTCACCCAGATCGTCAGCGCCGAG
GCCTGGGGTAGAGCAGACTGTGGCTTTACCTCGGTGTCCTACCAGCAAGGGGTCCTGTCTGCCACCATCCTCTATGAGATC
CTGCTAGGGAAGGCCACCCTGTATGCTGTGCTGGTCAGCGCCCTTGTGTTGATGGCCATGGTCAAGAGAAAGGATTTCTG
A (SEQ ID NO. 181)
Cluster h Block V
MGSRLLCWVLLCLLGAGPVKAGVTQTPRYLIKTRGQQVTLSCSPISGHRSVSWYQQTPGQGLQFLFEYFSETQRNKGNFPGRFS
GRQFSNSRSEMNVSTLELGDSALYL (SEQ ID NO. 182)
Seq-ID h.1b CASSLAGDQPQHF (SEQ ID NO. 172)
Seq-ID h.2b CASSLEGDRPQHF (SEQ ID NO. 173)
Cluster h Block J/C
GDGTRLSILEDLNKVFPPEVAVFEPSEAEISHTQKATLVCLATGFFPDHVELSWWVNGKEVHSGVSTDPQPLKEQPALNDSRYCL
SSRLRVSATFWQNPRNHFRCQVQFYGLSENDEWTQDRAKPVTQIVSAEAWGRADCGFTSVSYQQGVLSATILYEILLGKATLY
AVLVSALVLMAMVKRKDF* (SEQ ID NO. 183)
Alpha-chain, TRA: TRAV22, TRAJ26, TRAC*01
Cluster h Block V
ATGAAGAGGATATTGGGAGCTCTGCTGGGGCTCTTGAGTGCCCAGGTTTGCTGTGTGAGAGGAATACAAGTGGAGCAGA
GTCCTCCAGACCTGATTCTCCAGGAGGGAGCCAATTCCACGCTGCGGTGCAATTTTTCTGACTCTGTGAACAATTTGCAGT
GGTTTCATCAAAACCCTTGGGGACAGCTCATCAACCTGTTTTACATTCCCTCAGGGACAAAACAGAATGGAAGATTAAGCG
CCACGACTGTCGCTACGGAACGCTACAGCTTATTGTACATTTCCTCTTCCCAGACCACAGACTCAGGCGTTTATTTC (SEQ
ID NO. 184)
Seq-ID h.1a TGTGCTGTCCGCTATGGTCAGAATTTTGTCTTT (SEQ ID NO. 185)
Seq-ID h.2a TGTGCTGTCCGCTATGGTCAGAATTTTGTCTTT (SEQ ID NO. 186)
Cluster h Block J/C
GGTCCCGGAACCAGATTGTCCGTGCTGCCCTATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 187)
Cluster h Block V
MKRILGALLGLLSAQVCCVRGIQVEQSPPDLILQEGANSTLRCNFSDSVNNLQWFHQNPWGQLINLFYIPSGTKQNGRLSATTV
ATERYSLLYISSSQTTDSGVYF (SEQ ID NO. 188)
Seq-ID h.1a CAVRYGQNFVF (SEQ ID NO. 175)
Seq-ID h.2a CAVRYGQNFVF (SEQ ID NO. 176)
Cluster h Block J/C
GPGTRLSVLPYIQNPDPAVYQLRDSKSSDKSVCLFTDFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGFRILLLKVAGFNLLMTLRLWSS* (SEQ ID NO. 189)

TABLE 9
Cluster ID i:

| | | CDR3 beta: | CDR3 alpha: | | | |
|---|---|---|---|---|---|---|
| SEQ-ID | Pat-ID | Amino acid seq. | Amino acid seq. | HLA_A_I | HLA_B_I | HLA_C_I |
| i.1 | P62 | CASSSSGAGDQPQHF | CAASETSYDKVIF | A*01:01 | B*08:01 | C*07:01 |
| | | (SEQ ID NO. 190) | (SEQ ID NO. 193) | | | |
| i.2 | P62 | CASSSGTGGNQPQHF | | A*01:01 | B*08:01 | C*07:01 |
| | | (SEQ ID NO. 191) | | | | |
| i.3 | P86 | CASSSEGAGQPQHF | | | | |
| | | (SEQ ID NO. 192) | | | | |
| i.4 | P86 | | CAASSTSYDKVIF | | | |
| | | | (SEQ ID NO. 194) | | | |

### Cluster ID i:

Cluster i is associated with HLA-A*01:01, HLA-B*08:01, HLA-C*07:01
Beta chain, TRB:
TRBV7-9, TRBJ1-5, TRBC2*01
Cluster i Block V
ATGGGCACCAGCCTCCTCTGCTGGATGGCCCTGTGTCTCCTGGGGGCAGATCACGCAGATACTGGAGTCTCCCAGAACCC
CAGACACAAGATCACAAAGAGGGGACAGAATGTAACTTTCAGGTGTGATCCAATTTCTGAACACAACCGCCTTTATTGGTA
CCGACAGACCCTGGGGCAGGGCCCAGAGTTTCTGACTTACTTCCAGAATGAAGCTCAACTAGAAAAATCAAGGCTGCTCA
GTGATCGGTTCTCTGCAGAGAGGCCTAAGGGATCTTTCTCCACCTTGGAGATCCAGCGCACAGAGCAGGGGGACTCGGCC
ATGTATCTCTGTGCCAGCAGCTCCTCCGGGGCCGGGGATCAGCCCCAGCATTTT (SEQ ID NO. 195)
Seq-ID i.1b TGTGCCAGCAGCTCCTCCGGGGCCGGGGATCAGCCCCAGCATTTT (SEQ ID NO. 196)
Seq-ID i.2b TGTGCCAGCAGCTCTGGGACAGGGGGCAATCAGCCCCAGCATTTT (SEQ ID NO. 197)
Seq-ID i.3b TGTGCCAGCAGCTCC---GAGGGGGCCGGTCAGCCCCAGCATTTT (SEQ ID NO. 198)
Cluster i Block J/C
GGTGATGGGACTCGACTCTCCATCCTAGAGGACCTGAACAAGGTGTTCCCACCCGAGGTCGCTGTGTTTGAGCCATCAGA
AGCAGAGATCTCCCACACCCAAAAGGCCACACTGGTGTGCCTGGCCACAGGCTTCTTCCCTGACCACGTGGAGCTGAGCT
GGTGGGTGAATGGGAAGGAGGTGCACAGTGGGGTCAGCACGGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCCGTCACCCAGATCGTCAGCGCCGAG
GCCTGGGGTAGAGCAGACTGTGGCTTTACCTCGGTGTCCTACCAGCAAGGGGTCCTGTCTGCCACCATCCTCTATGAGATC
CTGCTAGGGAAGGCCACCCTGTATGCTGTGCTGGTCAGCGCCCTTGTGTTGATGGCCATGGTCAAGAGAAAGGATTTCTG
A (SEQ ID NO. 199)
Cluster i Block V
MGTSLLCWMALCLLGADHADTGVSQNPRHKITKRGQNVTFRCDPISEHNRLYWYRQTLGQGPEFLTYFQNEAQLEKSRLLSDR
FSAERPKGSFSTLEIQRTEQGDSAMYL (SEQ ID NO. 200)
Seq-ID i.1b CASSSSGAGDQPQHF (SEQ ID NO. 190)
Seq-ID i.2b CASSSGTGGNQPQHF (SEQ ID NO. 191)
Seq-ID i.3b CASSSEGAG-QPQHF (SEQ ID NO. 192)
Cluster i Block J/C
GDGTRLSILEDLNKVFPPEVAVFEPSEAEISHTQKATLVCLATGFFPDHVELSWWVNGKEVHSGVSTDPQPLKEQPALNDSRYCL
SSRLRVSATFWQNPRNHFRCQVQFYGLSENDEWTQDRAKPVTQIVSAEAWGRADCGFTSVSYQQGVLSATILYEILLGKATLY
AVLVSALVLMAMVKRKDF* (SEQ ID NO. 201)
Alpha-chain, TRA: TRAV13-1, TRAJ50, TRAC*01
Cluster i Block V
ATGACATCCATTCGAGCTGTATTTATATTCCTGTGGCTGCAGCTGGACTTGGTGAATGGAGAGAATGTGGAGCAGCATCCT
TCAACCCTGAGTGTCCAGGAGGGAGACAGCGCTGTTATCAAGTGTACTTATTCAGACAGTGCCTCAAACTACTTCCCTTGG
TATAAGCAAGAACTTGGAAAAAGACCTCAGCTTATTATAGACATTCGTTCAAATGTGGGCGAAAAGAAAGACCAACGAAT
TGCTGTTACATTGAACAAGACAGCCAAACATTTCTCCCTGCACATCACAGAGACCCAACCTGAAGACTCGGCTGTCTACTTC
(SEQ ID NO. 202)
Seq-ID i.1a TGTGCAGCAAGTGAAACCTCCTACGACAAGGTGATATTT (SEQ ID NO. 203)
Seq-ID i.4a TGTGCAGCAAGTAGCACCTCCTACGACAAGGTGATATTT (SEQ ID NO. 204)
Cluster i Block J/C
GGGCCAGGGACAAGCTTATCAGTCATTCCAAATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 205)
Cluster i Block V
MTSIRAVFIFLWLQLDLVNGENVEQHPSTLSVQEGDSAVIKCTYSDSASNYFPWYKQELGKRPQLIIDIRSNVGEKKDQRIAVTLN
KTAKHFSLHITETQPEDSAVYF (SEQ ID NO. 206)
Seq-ID i.1a CAASETSYDKVIF (SEQ ID NO. 193)
Seq-ID i.4a CAASSTSYDKVIF (SEQ ID NO. 194)
Cluster i Block J/C
GPGTSLSVIPNIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGENLLMTLRLWSS* (SEQ ID NO. 207)

TABLE 10
Cluster ID j:

| | | CDR3 beta: | CDR3 alpha: | |
|---|---|---|---|---|
| SEQ-ID | Pat-ID | Amino acid seq. | Amino acid seq. | HLA_A_I |
| j.1 | P82 | CSARVGVGNTIYF | CAEDNNARLMF | A*02:01 |
| | | (SEQ ID NO. 208) | (SEQ ID NO. 212) | |
| j.2 | P87 | CSARVGVGNTIYF | CAEDLNARLMF | A*02:01 |
| | | (SEQ ID NO. 209) | (SEQ ID NO. 213) | |
| j.3 | P86 | CSARDQVGNTIYF | | A*02:01 |
| | | (SEQ ID NO. 210) | | |
| j.4 | P93 | CSARAGVGNTIYF | | |
| | | (SEQ ID NO. 211) | | |
| j.5 | PANC10 | CSARDQTGNTIYF | CAEDENARLMF | A*02:01 |
| | | (SEQ ID NO. 478) | (SEQ ID NO. 513) | |

Cluster j is associated with HLA-A*02:01
Beta chain, TRB: TRBV20-1, TRBJ1-3, TRBC1*01
Cluster j Block V
ATGCTGCTGCTTCTGCTGCTTCTGGGGCCAGGCTCCGGGCTTGGTGCTGTCGTCTCTCAACATCCGAGCAGGGTTATCTGT
AAGAGTGGAACCTCTGTGAAGATCGAGTGCCGTTCCCTGGACTTTCAGGCCACAACTATGTTTTGGTATCGTCAGTTCCCG
AAACAGAGTCTCATGCTGATGGCAACTTCCAATGAGGGCTCCAAGGCCACATACGAGCAAGGCGTCGAGAAGGACAAGT
TTCTCATCAACCATGCAAGCCTGACCTTGTCCACTCTGACAGTGACCAGTGCCCATCCTGAAGACAGCAGCTTCTACATC
(SEQ ID NO. 214)
Seq-ID j.1b TGCAGTGCTAGAGTAGGGGTTGGAAACACCATATATTTT (SEQ ID NO. 215)
Seq-ID j.2b TGCAGTGCTAGAGTTGGGGTTGGAAACACCATATATTTT (SEQ ID NO. 216)
Seq-ID j.3b TGCAGTGCTAGAGACCAGGTTGGAAACACCATATATTTT (SEQ ID NO. 217)
Seq-ID j.4b TGCAGTGCTAGGGCAGGGGTAGGAAACACCATATATTTT (SEQ ID NO. 218)
Seq-ID j.5b TGCAGTGCTAGAGATCAGACCGGAAACACCATATATTTT (SEQ ID NO. 479)
Cluster j Block J/C
GGAGAGGGAAGTTGGCTCACTGTTGTAGAGGACCTGAACAAGGTGTTCCCACCCGAGGTCGCTGTGTTTGAGCCATCAGA
AGCAGAGATCTCCCACACCCAAAAGGCCACACTGGTGTGCCTGGCCACAGGCTTCTTCCCTGACCACGTGGAGCTGAGCT
GGTGGGTGAATGGGAAGGAGGTGCACAGTGGGGTCAGCACGGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCCGTCACCCAGATCGTCAGCGCCGAG
GCCTGGGGTAGAGCAGACTGTGGCTTTACCTCGGTGTCCTACCAGCAAGGGGTCCTGTCTGCCACCATCCTCTATGAGATC
CTGCTAGGGAAGGCCACCCTGTATGCTGTGCTGGTCAGCGCCCTTGTGTTGATGGCCATGGTCAAGAGAAAGGATTTCTG
A (SEQ ID NO. 219)
Cluster j Block V
MLLLLLLLGPGSGLGAVVSQHPSRVICKSGTSVKIECRSLDFQATTMFWYRQFPKQSLMLMATSNEGSKATYEQGVEKDKFLIN
HASLTLSTLTVTSAHPEDSSFYI (SEQ ID NO. 220)
Seq-ID j.1b CSARVGVGNTIYF (SEQ ID NO. 208)
Seq-ID j.2b CSARVGVGNTIYF (SEQ ID NO. 209)
Seq-ID j.3b CSARDQVGNTIYF (SEQ ID NO. 210)
Seq-ID j.4b CSARAGVGNTIYF (SEQ ID NO. 211)
Seq-ID j.5b CSARDQTGNTIYF (SEQ ID NO. 478)
Cluster j Block J/C
GEGSWLTVVEDLNKVFPPEVAVFEPSEAEISHTQKATLVCLATGFFPDHVELSWWVNGKEVHSGVSTDPQPLKEQPALNDSRY
CLSSRLRVSATFWQNPRNHFRCQVQFYGLSENDEWTQDRAKPVTQIVSAEAWGRADCGFTSVSYQQGVLSATILYEILLGKATL
YAVLVSALVLMAMVKRKDF* (SEQ ID NO. 221)
Alpha-chain, TRA: TRAV5, TRAJ31, TRAC*01
Cluster j Block V
ATGAAGACATTTGCTGGATTTTCGTTCCTGTTTTTGTGGCTGCAGCTGGACTGTATGAGTAGAGGAGAGGATGTGGAGCA
GAGTCTTTTCCTGAGTGTCCGAGAGGGAGACAGCTCCGTTATAAACTGCACTTACACAGACAGCTCCTCCACCTACTTATAC
TGGTATAAGCAAGAACCTGGAGCAGGTCTCCAGTTGCTGACGTATATTTTTTCAAATATGGACATGAAACAAGACCAAAGA
CTCACTGTTCTATTGAATAAAAAGGATAAACATCTGTCTCTGCGCATTGCAGACACCCAGACTGGGGACTCAGCTATCTACT
TC (SEQ ID NO. 222)
Seq-ID j.1a TGTGCAGAGGATAACAATGCCAGACTCATGTTT (SEQ ID NO. 223)
Seq-ID j.2a TGTGCAGAGGATCTTAATGCCAGACTCATGTTT (SEQ ID NO. 224)
Seq-ID j.5a TGTGCAGAGGACGAAAATGCCAGACTCATGTTT (SEQ ID NO. 480)
Cluster j Block J/C
GGAGATGGAACTCAGCTGGTGGTGAAGCCCAATATCCAGAACCCTGACCCTGCCGTGTACCAGCTGAGAGACTCTAAATC
CAGTGACAAGTCTGTCTGCCTATTCACCGATTTTGATTCTCAAACAAATGTGTCACAAAGTAAGGATTCTGATGTGTATATC
ACAGACAAAACTGTGCTAGACATGAGGTCTATGGACTTCAAGAGCAACAGTGCTGTGGCCTGGAGCAACAAATCTGACTT
TGCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 225)
Cluster j Block V
MKTFAGFSFLFLWLQLDCMSRGEDVEQSLFLSVREGDSSVINCTYTDSSSTYLYWYKQEPGAGLQLLTYIFSNMDMKQDQRLT
VLLNKKDKHLSLRIADTQTGDSAIYF (SEQ ID NO. 226)
Seq-ID j.1a CAEDNNARLMF (SEQ ID NO. 212)
Seq-ID j.2a CAEDLNARLMF (SEQ ID NO. 213)
Seq-ID j.5a CAEDENARLMF (SEQ ID NO. 513)
Cluster j Block J/C
GDGTQLVVKPNIQNPDPAVYQLRDSKSSDKSVCLFTDFDSQTNVSQSKDSDVYITDKTVLDMRSMDFKSNSAVAWSNKSDFA
CANAFNNSIIPEDTFFPSPESSCDVKLVEKSFETDTNLNFQNLSVIGFRILLLKVAGFNLLMITLRLWSS* (SEQ ID NO.
227)

TABLE 11
Cluster ID k:

| | | CDR3 beta: | CDR3 alpha: | |
|---|---|---|---|---|
| SEQ-ID | Pat-ID | Amino acid seq. | Amino acid seq. | HLA_A_I |
| k.1 | P93 | CASSTGAYGYTF | CAFMTNAGGTSYGKLTF | |
| | | (SEQ ID NO. 228) | (SEQ ID NO. 232) | |
| k.2 | P91 | CASSIGIYGYTF | CAYSPNAGGTSYGKLTF | A*02:01 |
| | | (SEQ ID NO. 229) | (SEQ ID NO. 233) | |
| k.3 | P93 | CASSIGWHGYTf | CAFMLNAGGTSYGKLTF | |
| | | (SEQ ID NO. 230) | (SEQ ID NO. 234) | |
| k.4 | P93 | CASSQGVYGYTF | CAYHLSAGGTSYGKLTF | |
| | | (SEQ ID NO. 231) | (SEQ ID NO. 235) | |

### Cluster ID k:

Cluster k is associated with HLA-A*02:01
Beta chain, TRB: TRBV19, TRBJ1-2, TRBC1*01
Cluster k Block V
ATGAGCAACCAGGTGCTCTGCTGTGTGGTCCTTTGTTTCCTGGGAGCAAACACCGTGGATGGTGGAATCACTCAGTCCCCA
AAGTACCTGTTCAGAAAGGAAGGACAGAATGTGACCCTGAGTTGTGAACAGAATTTGAACCACGATGCCATGTACTGGTA
CCGACAGGACCCAGGGCAAGGGCTGAGATTGATCTACTACTCACAGATAGTAAATGACTTTCAGAAAGGAGATATAGCTG
AAGGGTACAGCGTCTCTCGGGAGAAGAAGGAATCCTTTCCTCTCACTGTGACATCGGCCCAAAAGAACCCGACAGCTTTCT
ATCTC (SEQ ID NO. 236)
Seq-ID k.1b TGTGCCAGTAGTACAGGGGCCTATGGCTACACCTTC (SEQ ID NO. 237)
Seq-ID k.2b TGTGCCAGTAGTATAGGGATCTATGGCTACACCTTC (SEQ ID NO. 238)
Seq-ID k.3b TGTGCCAGTAGTATCGGTTGGCATGGCTACACCTTC (SEQ ID NO. 239)
Seq-ID k.4b TGTGCCAGTAGCCAGGGGGTCTATGGCTACACCTTC (SEQ ID NO. 240)
Cluster k Block J/C
GGTTCGGGGACCAGGTTAACCGTTGTAGAGGACCTGAACAAGGTGTTCCCACCCGAGGTCGCTGTGTTTGAGCCATCAGA
AGCAGAGATCTCCCACACCCAAAAGGCCACACTGGTGTGCCTGGCCACAGGCTTCTTCCCTGACCACGTGGAGCTGAGCT
GGTGGGTGAATGGGAAGGAGGTGCACAGTGGGGTCAGCACGGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCCGTCACCCAGATCGTCAGCGCCGAG
GCCTGGGGTAGAGCAGACTGTGGCTTTACCTCGGTGTCCTACCAGCAAGGGGTCCTGTCTGCCACCATCCTCTATGAGATC
CTGCTAGGGAAGGCCACCCTGTATGCTGTGCTGGTCAGCGCCCTTGTGTTGATGGCCATGGTCAAGAGAAAGGATTTCTG
A (SEQ ID NO. 241)
Cluster k Block V
MSNQVLCCVVLCFLGANTVDGGITQSPKYLFRKEGQNVTLSCEQNLNHDAMYWYRQDPGQGLRLIYYSQIVNDFQKGDIAEG
YSVSREKKESFPLTVTSAQKNPTAFYL (SEQ ID NO. 242)
Seq-ID k.1b CASSTGAYGYTF (SEQ ID NO. 228)
Seq-ID k.2b CASSIGIYGYTF (SEQ ID NO. 229)
Seq-ID k.3b CASSIGWHGYTF (SEQ ID NO. 230)
Seq-ID k.4b CASSQGVYGYTF (SEQ ID NO. 231)
Cluster k Block J/C
GSGTRLTVVEDLNKVFPPEVAVFEPSEAEISHTQKATLVCLATGFFPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSVSYQQGVLSATILYEILLGKATLY
AVLVSALVLMAMVKRKDF* (SEQ ID NO. 243)
Alpha-chain, TRA: TRAV38-1_38-2/DV8, TRAJ52, TRAC*01
Cluster k Block V
ATGACACGAGTTAGCTTGCTGTGGGCAGTCGTGGTCTCCACCTGTCTTGAATCCGGCATGGCCCAGACAGTCACTCAGTCT
CAACCAGAGATGTCTGTGCAGGAGGCAGAGACTGTGACCCTGAGTTGCACATATGACACCAGTGAGAATAATTATTATTT
GTTCTGGTACAAGCAGCCTCCCAGCAGGCAGATGATTCTCGTTATTCGCCAAGAAGCTTATAAGCAACAGAATGCAACGG
AGAATCGTTTCTCTGTGAACTTCCAGAAAGCAGCCAAATCCTTCAGTCTCAAGATCTCAGACTCACAGCTGGGGGACACTG
CGATGTATTTC (SEQ ID NO. 244)
Seq-ID k.1a TGTGCTTTCATGACCAATGCTGGTGGTACTAGCTATGGAAAGCTGACATTT (SEQ ID NO. 245)
Seq-ID k.2a TGTGCTTATAGCCCCAATGCTGGTGGTACTAGCTATGGAAAGCTGACATTT (SEQ ID NO. 246)
Seq-ID k.3a TGTGCTTTCATGCTTAATGCTGGTGGTACTAGCTATGGAAAGCTGACATTT (SEQ ID NO. 247)
Seq-ID k.4a TGTGCTTATCACCTCAGTGCTGGTGGTACTAGCTATGGAAAGCTGACATTT (SEQ ID NO. 248)
Cluster k Block J/C
GGACAAGGGACCATCTTGACTGTCCATCCAAATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 249)
Cluster k Block V
MTRVSLLWAVVVSTCLESGMAQTVTQSQPEMSVQEAETVTLSCTYDTSENNYYLFWYKQPPSRQMILVIRQEAYKQQNATEN
RFSVNFQKAAKSFSLKISDSQLGDTAMYF (SEQ ID NO. 250)
Seq-ID k.1a CAFMTNAGGTSYGKLTF (SEQ ID NO. 232)
Seq-ID k.2a CAYSPNAGGTSYGKLTF (SEQ ID NO. 233)
Seq-ID k.3a CAFMLNAGGTSYGKLTF (SEQ ID NO. 234)
Seq-ID k.4a CAYHLSAGGTSYGKLTF (SEQ ID NO. 235)
Cluster k Block J/C
GQGTILTVHPNIQNPDPAVYQLRDSKSSDKSVCLFTDFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGFRILLLKVAGFNLLMTLRLWSS* (SEQ ID NO. 251)

TABLE 12
Cluster ID I:

| | | CDR3 beta: | CDR3 alpha: | |
|---|---|---|---|---|
| SEQ-ID | Pat-ID | Amino acid seq. | Amino acid seq. | HLA_I |
| I.1 | P91 | CASSSDRGSTDTQYF | CAVRDSNYQLIW | Not |
| | | (SEQ ID NO. 252) | (SEQ ID NO. 261) | conclusive |
| I.2 | P84 | CASSERRGDTDTQYF | CASMDSNYQLIW | Not |
| | | (SEQ ID NO. 253) | (SEQ ID NO. 262) | conclusive |
| I.3 | P92 | CASSPGTGDTDTQYF | CAVMDSNYQLIW | Not |
| | | (SEQ ID NO. 254) | (SEQ ID NO. 263) | conclusive |
| I.4 | P92 | CASSPGTGDTDTQYF | CAVMDSNYQLIW | Not |
| | | (SEQ ID NO. 255) | (SEQ ID NO. 264) | conclusive |
| I.5 | P73 | CASSERAGGTDTQYF | CASSDSNYQLIW | Not |
| | | (SEQ ID NO. 256) | (SEQ ID NO. 265) | conclusive |
| I.6 | P62 | CASSEQGTDTQYF | CSCMDSNYQLIW | Not |
| | | (SEQ ID NO. 257) | (SEQ ID NO. 266) | conclusive |
| I.7 | P84 | CASSETSDTDTQYF | CAVRDSNYQLIW | Not |
| | | (SEQ ID NO. 258) | (SEQ ID NO. 267) | conclusive |
| I.8 | P85 | CASSTGGGTDTQYF | CAVMDSNYQLIW | Not |
| | | (SEQ ID NO. 259) | (SEQ ID NO. 268) | conclusive |
| I.9 | P86 | CASSDSRGGTDTQYF | CAVMDSNYQLIW | Not |
| | | (SEQ ID NO. 260) | (SEQ ID NO. 269) | conclusive |
| I.10 | PANC06 | CASSDSSGGQETQYF | CAVMDSNYQLIW | Not |
| | | (SEQ ID NO. 481) | (SEQ ID NO. 484) | conclusive |
| I.11 | PANC07 | CASSDSSGGTDTQYF | CAVRDSNYQLIW | Not |
| | | (SEQ ID NO. 482) | (SEQ ID NO. 485) | conclusive |
| I.12 | PANC09 | CASSDSSGGTDTQYF | CAVRDSNYQLIW | Not |
| | | (SEQ ID NO. 483) | (SEQ ID NO. 486) | conclusive |

### Cluster ID I:

Cluster I has no conclusive MHC I/II association
Beta chain, TRB: TRBV6-4/3-1, TRBJ2-3, TRBC2*01
Cluster I Block V
ATGAGAATCAGGCTCCTGTGCTGTGTGGCCTTTTCTCTCCTGTGGGCAGGTCCAGTGATTGCTGGGATCACCCAGGCACCA
ACATCTCAGATCCTGGCAGCAGGACGGCGCATGACACTGAGATGTACCCAGGATATGAGACATAATGCCATGTACTGGTA
TAGACAAGATCTAGGACTGGGGCTAAGGCTCATCCATTATTCAAATACTGCAGGTACCACTGGCAAAGGAGAAGTCCCTG
ATGGTTATAGTGTCTCCAGAGCAAACACAGATGATTTCCCCCTCACGTTGGCGTCTGCTGTACCCTCTCAGACATCTGTGTA
CTTC (SEQ ID NO. 270)
Seq-ID I.1b TGTGCCAGCAGCAGCGACAGGGGAAGCACAGATACGCAGTATTTT (SEQ ID NO. 271)
Seq-ID I.2b TGTGCCAGCAGTGAGAGGCGGGGGGACACAGATACGCAGTATTTT (SEQ ID NO. 272)
Seq-ID I.3b TGTGCCAGCAGCCCCGGGACAGGGGACACAGATACGCAGTATTTT (SEQ ID NO. 273)
Seq-ID I.4b TGTGCCAGCAGCCCCGGGACAGGGGACACAGATACGCAGTATTTT (SEQ ID NO. 274)
Seq-ID I.5b TGTGCCAGCAGTGAGAGAGCGGGGGGCACAGATACGCAGTATTTT (SEQ ID NO. 275)
Seq-ID I.6b TGTGCCAGCAGTGAACAA------GGCACAGATACGCAGTATTTT (SEQ ID NO. 276)
Seq-ID I.7b TGTGCCAGCAGTGAAACTAGC---GACACAGATACGCAGTATTTT (SEQ ID NO. 277)
Seq-ID I.8b TGTGCCAGCAGTACGGGGGGC---GGCACAGATACGCAGTATTTT (SEQ ID NO. 278)
Seq-ID I.9b TGTGCCAGCAGTGACTCGCGGGGGGGCACAGATACGCAGTATTTT (SEQ ID NO. 279)
Seq-ID I.10b TGTGCCAGCAGTGACTCTAGCGGGGGGCAAGAGACCCAGTACTTC (SEQ ID NO. 487)
Seq-ID I.11b TGTGCCAGCAGTGACTCTAGCGGGGGCACAGATACGCAGTATTTT (SEQ ID NO. 488)
Seq-ID I.12b TGTGCCAGCAGTGACTCCTCAGGGGGCACAGATACGCAGTATTTT (SEQ ID NO. 489)
Cluster I Block J/C
GGCCCAGGCACCCGGCTGACAGTGCTCGAGGACCTGAAAAACGTGTTCCCACCCAAGGTCGCTGTGTTTGAGCCATCAGA AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 280)
Cluster I Block V
MRIRLLCCVAFSLLWAGPVIAGITQAPTSQILAAGRRMTLRCTQDMRHNAMYWYRQDLGLGLRLIHYSNTAGTTGKGEVPDG
YSVSRANTDDFPLTLASAVPSQTSVYF (SEQ ID NO. 281)
Seq-ID I.1b CASSSDRGSTDTQYF (SEQ ID NO. 252)
Seq-ID I.2b CASSERRGDTDTQYF (SEQ ID NO. 253)
Seq-ID I.3b CASSPGTGDTDTQYF (SEQ ID NO. 254)
Seq-ID I.4b CASSPGTGDTDTQYF (SEQ ID NO. 255)
Seq-ID I.5b CASSERAGGTDTQYF (SEQ ID NO. 256)
Seq-ID I.6b CASSEQG--TDTQYF (SEQ ID NO. 257)
Seq-ID I.7b CASSETSD-TDTQYF (SEQ ID NO. 258)
Seq-ID I.8b CASS-TGGGTDTQYF (SEQ ID NO. 259)
Seq-ID I.9b CASSDSRGGTDTQYF (SEQ ID NO. 260)
Seq-ID I.10b CASSDSSGGQETQYF (SEQ ID NO. 481)
Seq-ID I.11b CASSDSSGGTDTQYF (SEQ ID NO. 482)
Seq-ID I.12b CASSDSSGGTDTQYF (SEQ ID NO. 483)
Cluster I Block J/C
GPGTRLTVLEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 282)
Alpha-chain, TRA: TRAV1-2, TRAJ33, TRAC*01
Cluster I Block V
ATGTGGGGAGTTTTCCTTCTTTATGTTTCCATGAAGATGGGAGGCACTACAGGACAAAACATTGACCAGCCCACTGAGATG
ACAGCTACGGAAGGTGCCATTGTCCAGATCAACTGCACGTACCAGACATCTGGGTTCAACGGGCTGTTCTGGTACCAGCA
ACATGCTGGCGAAGCACCCACATTTCTGTCTTACAATGTTCTGGATGGTTTGGAGGAGAAAGGTCGTTTTTCTTCATTCCTT
AGTCGGTCTAAAGGGTACAGTTACCTCCTTTTGAAGGAGCTCCAGATGAAAGACTCTGCCTCTTACCTC (SEQ ID NO.
283)
Seq-ID I.1a TGTGCTGTGAGGGATAGCAACTATCAGTTAATCTGG (SEQ ID NO. 284)
Seq-ID I.2a TGTGCTTCCATGGATAGCAACTATCAGTTAATCTGG (SEQ ID NO. 285)
Seq-ID I.3a TGTGCTGTGATGGATAGCAACTATCAGTTAATCTGG (SEQ ID NO. 286)
Seq-ID I.4a TGTGCTGTGATGGATAGCAACTATCAGTTAATCTGG (SEQ ID NO. 287)
Seq-ID I.5a TGTGCCTCTTCCGATAGCAACTATCAGTTAATCTGG (SEQ ID NO. 288)
Seq-ID I.6a TGCTCGTGCATGGATAGCAACTATCAGTTAATCTGG (SEQ ID NO. 289)
Seq-ID I.7a TGTGCTGTGAGAGATAGCAACTATCAGTTAATCTGG (SEQ ID NO. 290)
Seq-ID I.8a TGTGCTGTCATGGATAGCAACTATCAGTTAATCTGG (SEQ ID NO. 291)
Seq-ID I.9a TGTGCTGTGATGGATAGCAACTATCAGTTAATCTGG (SEQ ID NO. 292)
Seq-ID I.10a TGTGCTGTGATGGATAGCAACTATCAGTTAATCTGG (SEQ ID NO. 490)
Seq-ID I.11a TGTGCTGTGAGAGATAGCAACTATCAGTTAATCTGG (SEQ ID NO. 491)
Seq-ID I.12a TGTGCTGTGAGAGATAGCAACTATCAGTTAATCTGG (SEQ ID NO. 492)
Cluster I Block J/C
GGCGCTGGGACCAAGCTAATTATAAAGCCAGATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 293)
Cluster I Block V
MWGVFLLYVSMKMGGTTGQNIDQPTEMTATEGAIVQINCTYQTSGFNGLFWYQQHAGEAPTFLSYNVLDGLEEKGRFSSFLS
RSKGYSYLLLKELQMKDSASYL (SEQ ID NO. 294)
Seq-ID I.1a CAVRDSNYQLIW (SEQ ID NO. 261)
Seq-ID I.2a CASMDSNYQLIW (SEQ ID NO. 262)
Seq-ID I.3a CAVMDSNYQLIW (SEQ ID NO. 263)
Seq-ID I.4a CAVMDSNYQLIW (SEQ ID NO. 264)
Seq-ID I.5a CASSDSNYQLIW (SEQ ID NO. 265)
Seq-ID I.6a CSCMDSNYQLIW (SEQ ID NO. 266)
Seq-ID I.7a CAVRDSNYQLIW (SEQ ID NO. 267)
Seq-ID I.8a CAVMDSNYQLIW (SEQ ID NO. 268)
Seq-ID I.9a CAVMDSNYQLIW (SEQ ID NO. 269)
Seq-ID I.10a CAVMDSNYQLIW (SEQ ID NO. 484)
Seq-ID I.11a CAVRDSNYQLIW (SEQ ID NO. 485)
Seq-ID I.12a CAVRDSNYQLIW (SEQ ID NO. 486)
Cluster I Block J/C
GAGTKLIIKPDIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGFNLLMTLRLWSS* (SEQ ID NO. 295)

TABLE 13
Cluster ID m:

| | | CDR3 beta: | CDR3 alpha: | |
|---|---|---|---|---|
| SEQ-ID | PAT-ID | Amino acid seq. | Amino acid seq. | HLA_I |
| m.1 | P92 | CASSDSTTDTQYF | CAVMDSNYQLIW | Not |
| | | (SEQ ID NO. 296) | (SEQ ID NO. 302) | conclusive |
| m.2 | P86 | CASSDSISTDTQYF | CAVVDSNYQLIW | Not |
| | | (SEQ ID NO. 297) | (SEQ ID NO. 303) | conclusive |
| m.3 | P84 | CASSDSAGGEDTQYF | CAVRDSNYQLIW | Not |
| | | (SEQ ID NO. 298) | (SEQ ID NO. 304) | conclusive |
| m.4 | P92 | CASSENQGADTQYF | CAPMNSNYQLIW | Not |
| | | (SEQ ID NO. 299) | (SEQ ID NO. 305) | conclusive |
| m.5 | P86 | CASSDSGGSADTQYF | CAVRDSNYQLIW | Not |
| | | (SEQ ID NO. 300) | (SEQ ID NO. 306) | conclusive |
| m.6 | P75 | CASSDGDSADTQYF | CAVKDSNYQLIW | Not |
| | | (SEQ ID NO. 301) | (SEQ ID NO. 307) | conclusive |
| m.7 | PANC05 | CASSDSADTDTQYF | CAVQDSNYQLIW | Not |
| | | (SEQ ID NO. 493) | (SEQ ID NO. 496) | conclusive |
| m.8 | PANC07 | CASSDGQGGTDTQYF | CAVKDSNYQLIW | Not |
| | | (SEQ ID NO. 494) | (SEQ ID NO. 497) | conclusive |
| m.9 | PANC07 | CASSDQVTDTQYF | CAVRDSNYQLIW | Not |
| | | (SEQ ID NO. 495) | (SEQ ID NO. 498) | conclusive |

### Cluster ID m:

Cluster m has no conclusive MHC I/II association
Beta chain, TRB: TRBV6-4, TRBJ2-3, TRBC2*01
Cluster m Block V
ATGAGAATCAGGCTCCTGTGCTGTGTGGCCTTTTCTCTCCTGTGGGCAGGTCCAGTGATTGCTGGGATCACCCAGGCACCA
ACATCTCAGATCCTGGCAGCAGGACGGCGCATGACACTGAGATGTACCCAGGATATGAGACATAATGCCATGTACTGGTA
TAGACAAGATCTAGGACTGGGGCTAAGGCTCATCCATTATTCAAATACTGCAGGTACCACTGGCAAAGGAGAAGTCCCTG
ATGGTTATAGTGTCTCCAGAGCAAACACAGATGATTTCCCCCTCACGTTGGCGTCTGCTGTACCCTCTCAGACATCTGTGTA
CTTC (SEQ ID NO. 308)
Seq-ID m.1b TGTGCCAGCAGTGACTCGACCA------CAGATACGCAGTATTTT (SEQ ID NO. 309)
Seq-ID m.2b TGTGCCAGCAGTGACTCGATCAGCA---CAGATACGCAGTATTTT (SEQ ID NO. 310)
Seq-ID m.3b TGTGCCAGCAGTGACTCCGCGGGGGGCGAAGATACGCAGTATTTT (SEQ ID NO. 311)
Seq-ID m.4b TGTGCCAGCAGTGAAAATCAGGGGG---CAGATACGCAGTATTTT (SEQ ID NO. 312)
Seq-ID m.5b TGTGCCAGCAGTGACTCCGGAGGGAGCGCAGATACGCAGTATTTT (SEQ ID NO. 313)
Seq-ID m.6b TGTGCCAGCAGTGACGGGGATTCGG---CAGATACGCAGTATTTT (SEQ ID NO. 314)
Seq-ID m.7b TGTGCCAGCAGTGACTCGGCTGACA---CAGATACGCAGTATTTT (SEQ ID NO. 499)
Seq-ID m.8b TGTGCCAGCAGTGACGGCCAGGGTGGAACAGATACGCAGTATTTT (SEQ ID NO. 500)
Seq-ID m.9b TGTGCCAGCAGTGACC---AGGTTA---CAGATACGCAGTATTTT (SEQ ID NO. 501)
Cluster m Block J/C
GGCCCAGGCACCCGGCTGACAGTGCTCGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 315)
Cluster m Block V
MRIRLLCCVAFSLLWAGPVIAGITQAPTSQILAAGRRMTLRCTQDMRHNAMYWYRQDLGLGLRLIHYSNTAGTTGKGEVPDG
YSVSRANTDDFPLTLASAVPSQTSVYF (SEQ ID NO. 316)
Seq-ID m.1b CASSDST--TDTQYF (SEQ ID NO. 296)
Seq-ID m.2b CASSDSI-STDTQYF (SEQ ID NO. 297)
Seq-ID m.3b CASSDSAGGEDTQYF (SEQ ID NO. 298)
Seq-ID m.4b CASSENQ-GADTOYF (SEQ ID NO. 299)
Seq-ID m.5b CASSDSGGSADTQYF (SEQ ID NO. 300)
Seq-ID m.6b CASSDGD-SADTQYF (SEQ ID NO. 301)
Seq-ID m.7b CASSDSAD-TDTQYF (SEQ ID NO. 493)
Seq-ID m.8b CASSDGQGGTDTQYF (SEQ ID NO. 494)
Seq-ID m.9b CASSDQV-TDTQYF (SEQ ID NO. 495)
Cluster m Block J/C
GPGTRLTVLEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 317)
Alpha-chain, TRA: TRAV1-2, TRAJ33, TRAC*01
Cluster m Block V
ATGTGGGGAGTTTTCCTTCTTTATGTTTCCATGAAGATGGGAGGCACTACAGGACAAAACATTGACCAGCCCACTGAGATG
ACAGCTACGGAAGGTGCCATTGTCCAGATCAACTGCACGTACCAGACATCTGGGTTCAACGGGCTGTTCTGGTACCAGCA
ACATGCTGGCGAAGCACCCACATTTCTGTCTTACAATGTTCTGGATGGTTTGGAGGAGAAAGGTCGTTTTTCTTCATTCCTT
AGTCGGTCTAAAGGGTACAGTTACCTCCTTTTGAAGGAGCTCCAGATGAAAGACTCTGCCTCTTACCTC (SEQ ID NO.
318)
Seq-ID m.1a TGTGCTGTGATGGATAGCAACTATCAGTTAATCTGG (SEQ ID NO. 319)
Seq-ID m.2a TGTGCTGTGGTGGATAGCAACTATCAGTTAATCTGG (SEQ ID NO. 320)
Seq-ID m.3a TGTGCTGTGAGAGATAGCAACTATCAGTTAATCTGG (SEQ ID NO. 321)
Seq-ID m.4a TGTGCCCCCATGAATAGCAACTATCAGTTAATCTGG (SEQ ID NO. 322)
Seq-ID m.5a TGTGCTGTGAGAGATAGCAACTATCAGTTAATCTGG (SEQ ID NO. 323)
Seq-ID m.6a TGTGCTGTGAAGGATAGCAACTATCAGTTAATCTGG (SEQ ID NO. 324)
Seq-ID m.7a TGTGCTGTGCAGGATAGCAACTATCAGTTAATCTGG (SEQ ID NO. 502)
Seq-ID m.8a TGTGCTGTGAAGGATAGCAACTATCAGTTAATCTGG (SEQ ID NO. 503)
Seq-ID m.9a TGTGCTGTGAGAGATAGCAACTATCAGTTAATCTGG (SEQ ID NO. 504)
Cluster m Block J/C
GGCGCTGGGACCAAGCTAATTATAAAGCCAGATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 325)
Cluster m Block V
MWGVFLLYVSMKMGGTTGQNIDQPTEMTATEGAIVQINCTYQTSGFNGLFWYQQHAGEAPTFLSYNVLDGLEEKGRFSSFLS
RSKGYSYLLLKELQMKDSASYL (SEQ ID NO. 326)
Seq-ID m.1a CAVMDSNYQLIW (SEQ ID NO. 302)
Seq-ID m.2a CAVVDSNYQLIW (SEQ ID NO. 303)
Seq-ID m.3a CAVRDSNYQLIW (SEQ ID NO. 304)
Seq-ID m.4a CAPMNSNYQLIW (SEQ ID NO. 305)
Seq-ID m.5a CAVRDSNYQLIW (SEQ ID NO. 306)
Seq-ID m.6a CAVKDSNYQLIW (SEQ ID NO. 307)
Seq-ID m.7a CAVQDSNYQLIW (SEQ ID NO. 496)
Seq-ID m.8a CAVKDSNYQLIW (SEQ ID NO. 497)
Seq-ID m.9a CAVRDSNYQLIW (SEQ ID NO. 498)
Cluster m Block J/C
GAGTKLIIKPDIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGFNLLMTLRLWSS* (SEQ ID NO. 327)

TABLE 14
Cluster ID n:

| | | CDR3 beta: | CDR3 alpha: | |
|---|---|---|---|---|
| SEQ-ID | PAT-ID | Amino acid seq. | Amino acid seq. | HLA_I |
| n.1 | P91 | CASSEVAGQGTGELFF | CAVRDSNYQLIW | Not |
| | | (SEQ ID NO. 328) | (SEQ ID NO. 334) | conclusive |
| n.2 | P62 | CASSAGDTGELFF | CATADSNYQLIW | Not |
| | | (SEQ ID NO. 329) | (SEQ ID NO. 335) | conclusive |
| n.3 | P84 | CASSEAGTGDTGELFF | CAVLDSNYQLIW | Not |
| | | (SEQ ID NO. 330) | (SEQ ID NO. 336) | conclusive |
| n.4 | P85 | CASSVGGGTGELFF | CAVRDSNYQLIW | Not |
| | | (SEQ ID NO. 331) | (SEQ ID NO. 337) | conclusive |
| n.5 | P91 | CASSDGSGGTGELFF | CAVSDSNYQLIW | Not |
| | | (SEQ ID NO. 332) | (SEQ ID NO. 338) | conclusive |
| n.6 | P92 | CASSDDAGGTGELFF | CAVRDSNYQLIW | Not |
| | | (SEQ ID NO. 333) | (SEQ ID NO. 339) | conclusive |

### Cluster ID n:

Cluster n has no conclusive MHC I/II association
Beta chain, TRB: TRBV6-4/6-1, TRBJ2-2, TRBC2*01
Cluster n Block V
ATGAGCATCGGGCTCCTGTGCTGTGTGGCCTTTTCTCTCCTGTGGGCAAGTCCAGTGAATGCTGGTGTCACTCAGACCCCA
AAATTCCAGGTCCTGAAGACAGGACAGAGCATGACACTGCAGTGTGCCCAGGATATGAACCATAACTCCATGTACTGGTA
TCGACAAGACCCAGGCATGGGACTGAGGCTGATTTATTACTCAGCTTCTGAGGGTACCACTGACAAAGGAGAAGTCCCCA
ATGGCTACAATGTCTCCAGATTAAACAAACGGGAGTTCTCGCTCAGGCTGGAGTCGGCTGCTCCCTCCCAGACATCTGTGT
ACTTC (SEQ ID NO. 340)
Seq-ID n.1b TGTGCCAGCAGTGAAGTAGCCGGACAGGGCACCGGGGAGCTGTTTTTT (SEQ ID NO. 341)
Seq-ID n.2b TGTGCCAGCAGCGCAGGG---------GACACCGGGGAGCTGTTTTTT (SEQ ID NO. 342)
Seq-ID n.3b TGTGCCAGCAGTGAAGCCGGGACAGGGGACACCGGGGAGCTGTTTTTT (SEQ ID NO. 343)
Seq-ID n.4b TGTGCCAGCAGTGTCGGA------GGGGGCACCGGGGAGCTGTTTTTT (SEQ ID NO. 344)
Seq-ID n.5b TGTGCCAGCAGTGACGGTAGCGGGGGG---ACCGGGGAGCTGTTTTTT (SEQ ID NO. 345)
Seq-ID n.6b TGTGCCAGCAGTGACGATGCGGGGGGG---ACCGGGGAGCTGTTTTTT (SEQ ID NO. 346)
Cluster n Block J/C
GGAGAAGGCTCTAGGCTGACCGTACTGGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 347)
Cluster n Block V
MSIGLLCCVAFSLLWASPVNAGVTQTPKFQVLKTGQSMTLQCAQDMNHNSMYWYRQDPGMGLRLIYYSASEGTTDKGEVP
NGYNVSRLNKREFSLRLESAAPSQTSVYF (SEQ ID NO. 348)
Seq-ID n.1b CASSEVAGQGTGELFF (SEQ ID NO. 328)
Seq-ID n.2b CASSAGD---TGELFF (SEQ ID NO. 329)
Seq-ID n.3b CASSEAGTGDTGELFF (SEQ ID NO. 330)
Seq-ID n.4b CASS-V-GGGTGELFF (SEQ ID NO. 331)
Seq-ID n.5b CASSDGSG-GTGELFF (SEQ ID NO. 332)
Seq-ID n.6b CASSDDAG-GTGELFF (SEQ ID NO. 333)
Cluster n Block J/C
GEGSRLTVLEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 349)
Alpha-chain, TRA: TRAV1-2, TRAJ33, TRAC*01
Cluster n Block V
ATGTGGGGAGTTTTCCTTCTTTATGTTTCCATGAAGATGGGAGGCACTACAGGACAAAACATTGACCAGCCCACTGAGATG
ACAGCTACGGAAGGTGCCATTGTCCAGATCAACTGCACGTACCAGACATCTGGGTTCAACGGGCTGTTCTGGTACCAGCA
ACATGCTGGCGAAGCACCCACATTTCTGTCTTACAATGTTCTGGATGGTTTGGAGGAGAAAGGTCGTTTTTCTTCATTCCTT
AGTCGGTCTAAAGGGTACAGTTACCTCCTTTTGAAGGAGCTCCAGATGAAAGACTCTGCCTCTTACCTC (SEQ ID NO.
350)
Seq-ID n.1a TGTGCTGTGAGGGATAGCAACTATCAGTTAATCTGG (SEQ ID NO. 351)
Seq-ID n.2a TGTGCTACTGCGGATAGCAACTATCAGTTAATCTGG (SEQ ID NO. 352)
Seq-ID n.3a TGTGCTGTCCTGGATAGCAACTATCAGTTAATCTGG (SEQ ID NO. 353)
Seq-ID n.4a TGTGCTGTGAGAGATAGCAACTATCAGTTAATCTGG (SEQ ID NO. 354)
Seq-ID n.5a TGTGCTGTGAGTGATAGCAACTATCAGTTAATCTGG (SEQ ID NO. 355)
Seq-ID n.6a TGTGCTGTGAGAGATAGCAACTATCAGTTAATCTGG (SEQ ID NO. 356)
Cluster n Block J/C
GGCGCTGGGACCAAGCTAATTATAAAGCCAGATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 357)
Cluster n Block V
MWGVFLLYVSMKMGGTTGQNIDQPTEMTATEGAIVQINCTYQTSGFNGLFWYQQHAGEAPTFLSYNVLDGLEEKGRESSFLS
RSKGYSYLLLKELQMKDSASYL (SEQ ID NO. 358)
Seq-ID n.1a CAVRDSNYQLIW (SEQ ID NO. 334)
Seq-ID n.2a CATADSNYQLIW (SEQ ID NO. 335)
Seq-ID n.3a CAVLDSNYQLIW (SEQ ID NO. 336)
Seq-ID n.4a CAVRDSNYQLIW (SEQ ID NO. 337)
Seq-ID n.5a CAVSDSNYQLIW (SEQ ID NO. 338)
Seq-ID n.6a CAVRDSNYQLIW (SEQ ID NO. 339)
Cluster n Block J/C
GAGTKLIIKPDIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGENLLMTLRLWSS* (SEQ ID NO. 359)

TABLE 15
Cluster ID o:

| | | CDR3 beta: | CDR3 alpha: | |
|---|---|---|---|---|
| SEQ-ID | PAT-ID | Amino acid seq. | Amino acid seq. | HLA_I |
| 0.1 | P86 | CASSEAGGSYNEQFF | CAVMDSNYQLIW | Not |
| | | (SEQ ID NO. 360) | (SEQ ID NO. 363) | conclusive |
| 0.2 | P86 | CASSEAGGSYNEQFF | CAVMDSNYQLIW | Not |
| | | (SEQ ID NO. 361) | (SEQ ID NO. 364) | conclusive |
| 0.3 | P85 | CASSESDGSYNEQFF | CAVGDSNYQLIW | Not |
| | | (SEQ ID NO. 362) | (SEQ ID NO. 365) | conclusive |

### Cluster ID o:

Cluster o has no conclusive MHC I/II association
Beta chain, TRB: TRBV6-1, TRBJ2-1, TRBC2*01
Cluster o Block V
ATGAGCATCGGGCTCCTGTGCTGTGTGGCCTTTTCTCTCCTGTGGGCAAGTCCAGTGAATGCTGGTGTCACTCAGACCCCA
AAATTCCAGGTCCTGAAGACAGGACAGAGCATGACACTGCAGTGTGCCCAGGATATGAACCATAACTCCATGTACTGGTA
TCGACAAGACCCAGGCATGGGACTGAGGCTGATTTATTACTCAGCTTCTGAGGGTACCACTGACAAAGGAGAAGTCCCCA
ATGGCTACAATGTCTCCAGATTAAACAAACGGGAGTTCTCGCTCAGGCTGGAGTCGGCTGCTCCCTCCCAGACATCTGTGT
ACTTC (SEQ ID NO. 366)
Seq-ID o.1b TGTGCCAGCAGTGAAGCGGGGGGCTCCTACAATGAGCAGTTCTTC (SEQ ID NO. 367)
Seq-ID o.2b TGTGCCAGCAGTGAAGCGGGGGGCTCCTACAATGAGCAGTTCTTC (SEQ ID NO. 368)
Seq-ID o.3b TGTGCCAGCAGTGAAAGCGACGGGAGCTACAATGAGCAGTTCTTC (SEQ ID NO. 369)
Cluster o Block J/C
GGGCCAGGGACACGGCTCACCGTGCTAGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 370)
Cluster o Block V
MSIGLLCCVAFSLLWASPVNAGVTQTPKFQVLKTGQSMTLQCAQDMNHNSMYWYRQDPGMGLRLIYYSASEGTTDKGEVP
NGYNVSRLNKREFSLRLESAAPSQTSVYF (SEQ ID NO. 371)
Seq-ID o.1b CASSEAGGSYNEQFF (SEQ ID NO. 360)
Seq-ID o.2b CASSEAGGSYNEQFF (SEQ ID NO. 361)
Seq-ID o.3b CASSESDGSYNEQFF (SEQ ID NO. 362)
Cluster o Block J/C
GPGTRLTVLEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG* (SEQ ID NO. 372)
Alpha-chain, TRA: TRAV1-2, TRAJ33, TRAC*01
Cluster o Block V
ATGTGGGGAGTTTTCCTTCTTTATGTTTCCATGAAGATGGGAGGCACTACAGGACAAAACATTGACCAGCCCACTGAGATG
ACAGCTACGGAAGGTGCCATTGTCCAGATCAACTGCACGTACCAGACATCTGGGTTCAACGGGCTGTTCTGGTACCAGCA
ACATGCTGGCGAAGCACCCACATTTCTGTCTTACAATGTTCTGGATGGTTTGGAGGAGAAAGGTCGTTTTTCTTCATTCCTT
AGTCGGTCTAAAGGGTACAGTTACCTCCTTTTGAAGGAGCTCCAGATGAAAGACTCTGCCTCTTACCTC (SEQ ID NO.
373)
Seq-ID o.1a TGTGCTGTGATGGATAGCAACTATCAGTTAATCTGG (SEQ ID NO. 374)
Seq-ID o.2a TGTGCTGTGATGGATAGCAACTATCAGTTAATCTGG (SEQ ID NO. 375)
Seq-ID o.3a TGTGCTGTGGGGGATAGCAACTATCAGTTAATCTGG (SEQ ID NO. 376)
Cluster o Block J/C
GGCGCTGGGACCAAGCTAATTATAAAGCCAGATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 377)
Cluster o Block V
MWGVFLLYVSMKMGGTTGQNIDQPTEMTATEGAIVQINCTYQTSGFNGLFWYQQHAGEAPTFLSYNVLDGLEEKGRESSFLS
RSKGYSYLLLKELQMKDSASYL (SEQ ID NO. 378)
Seq-ID o.1a CAVMDSNYQLIW SEQ ID NO. 363)
Seq-ID 0.2a CAVMDSNYQLIW SEQ ID NO. 364)
Seq-ID 0.3a CAVGDSNYQLIW SEQ ID NO. 365)
Cluster o Block J/C
GAGTKLIIKPDIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGENLLMTLRLWSS* (SEQ ID NO. 379)

TABLE 16
Cluster ID p:

| | | CDR3 beta: | CDR3 alpha: | |
|---|---|---|---|---|
| SEQ-ID | PAT-ID | Amino acid seq. | Amino acid seq. | HLA_I |
| p.1 | P86 | CASSDSRGGTDTQYF | CAVMDSNYQLIW | Not |
| | | (SEQ ID NO. 380) | (SEQ ID NO. 391) | conclusive |
| p.2 | P86 | CASSGYSGGGADTQYF | CAVMDSNYQLIW | Not |
| | | (SEQ ID NO. 381) | (SEQ ID NO. 392) | conclusive |
| p.3 | P86 | CASSDSGGSADTQYF | CAVRDSNYQLIW | Not |
| | | (SEQ ID NO. 382) | (SEQ ID NO. 393) | conclusive |
| p.4 | P93 | CASSGLAGGPDTQYF | CAVRDSNYQLIW | Not |
| | | (SEQ ID NO. 383) | (SEQ ID NO. 394) | conclusive |
| p.5 | P82 | CASSELGGGGTDTQYF | CAVRRDSNYQLIW | Not |
| | | (SEQ ID NO. 384) | (SEQ ID NO. 395) | conclusive |
| p.6 | P85 | CASSTGGGTDTQYF | CAVMDSNYQLIW | Not |
| | | (SEQ ID NO. 385) | (SEQ ID NO. 396) | conclusive |
| p.7 | P73 | CASSERAGGTDTQYF | CASSDSNYQLIW | Not |
| | | (SEQ ID NO. 386) | (SEQ ID NO. 397) | conclusive |
| p.8 | P84 | CASSDSAGGEDTQYF | CAVRDSNYQLIW | Not |
| | | (SEQ ID NO. 387) | (SEQ ID NO. 398) | conclusive |
| p.9 | P84 | CASSGGTETDTQYF | CAVRDSNYQLIW | Not |
| | | (SEQ ID NO. 388) | (SEQ ID NO. 399) | conclusive |
| p.10 | P92 | CASSGPTSGGPGDTQYF | CAVLDSNYQLIW | Not |
| | | (SEQ ID NO. 389) | (SEQ ID NO. 400) | conclusive |
| p.11 | P62 | CASSQTSGGGADTQYF | CAVRDSNYQLIW | Not |
| | | (SEQ ID NO. 390) | (SEQ ID NO. 401) | conclusive |
| p.12 | PANC06 | CASSELAGGPDTQYF | CAVRDSNYQLIW | Not |
| | | (SEQ ID NO. 505) | (SEQ ID NO. 507) | conclusive |
| p.13 | PANC07 | CASSELAGGPETQYF | CAVRDSNYQLIW | Not |
| | | (SEQ ID NO. 506) | (SEQ ID NO. 508) | conclusive |

### Cluster ID p:

Cluster p has no conclusive MHC I/II association
Beta chain, TRB: TRBV6-1/6-4, TRBJ2-3, TRBC2*01
Cluster p Block V
ATGAGCATCGGGCTCCTGTGCTGTGTGGCCTTTTCTCTCCTGTGGGCAAGTCCAGTGAATGCTGGTGTCACTCAGACCCCA
AAATTCCAGGTCCTGAAGACAGGACAGAGCATGACACTGCAGTGTGCCCAGGATATGAACCATAACTCCATGTACTGGTA
TCGACAAGACCCAGGCATGGGACTGAGGCTGATTTATTACTCAGCTTCTGAGGGTACCACTGACAAAGGAGAAGTCCCCA
ATGGCTACAATGTCTCCAGATTAAACAAACGGGAGTTCTCGCTCAGGCTGGAGTCGGCTGCTCCCTCCCAGACATCTGTGT
ACTTC (SEQ ID NO. 402)
Seq-ID p.1b TGTGCCAGCAGTGACTCGCGGGGGGGCACAGATACGCAGTATTTT (SEQ ID NO. 403)
Seq-ID p.2b TGTGCCAGCAGTGGGTATAGCGGGGGGGGGGCAGATACGCAGTATTTT (SEQ ID NO. 404)
Seq-ID p.3b TGTGCCAGCAGTGACTCCGGAGGGAGCGCAGATACGCAGTATTTT (SEQ ID NO. 405)
Seq-ID p.4b TGTGCCAGCAGTGGACTAGCGGGGGGGCCAGATACGCAGTATTTT (SEQ ID NO. 406)
Seq-ID p.5b TGTGCCAGCAGTGAATTGGGGGGGGGGGGAACAGATACGCAGTATTTT (SEQ ID NO. 407)
Seq-ID p.6b TGTGCCAGCAGTACGGGGGGCGGCACAGATACGCAGTATTTT (SEQ ID NO. 408)
Seq-ID p.7b TGTGCCAGCAGTGAGAGAGCGGGGGGCACAGATACGCAGTATTTT (SEQ ID NO. 409)
Seq-ID p.8b TGTGCCAGCAGTGACTCCGCGGGGGGCGAAGATACGCAGTATTTT (SEQ ID NO. 410)
Seq-ID p.9b TGTGCCAGCAGTGGAGGGACCGAGACAGATACGCAGTATTTT (SEQ ID NO. 411)
Seq-ID p.10b TGTGCCAGCAGTGGCCCGACTAGCGGGGGGCCGGGAGATACGCAGTATTTT (SEQ ID NO. 412)
Seq-ID p.11b TGTGCCAGCAGTCAAACTAGCGGGGGGGGTGCAGATACGCAGTATTTT (SEQ ID NO. 413)
Seq-ID p.12b TGTGCCAGCAGTGAACTAGCGGGGGGGCCAGATACGCAGTATTTT (SEQ ID NO. 510)
Seq-ID p.13b TGTGCCAGCAGTGAATTAGCGGGGGGGCCAGAGACCCAGTACTTC (SEQ ID NO. 511)
Cluster p Block J/C
GGCCCAGGCACCCGGCTGACAGTGCTCGAGGACCTGAAAAACGTGTTCCCACCCAAGGTCGCTGTGTTTGAGCCATCAGA
AGCAGAGATCTCCCACACCCAAAAGGCCACACTGGTGTGCCTGGCCACAGGCTTCTACCCCGACCACGTGGAGCTGAGCT
GGTGGGTGAATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTTCCGCTGTCAAG
TCCAGTTCTACGGGCTCTCGGAGAATGACGAGTGGACCCAGGATAGGGCCAAACCTGTCACCCAGATCGTCAGCGCCGAG
GCCTGGGGTAGAGCAGACTGTGGCTTCACCTCCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCTGATGGCCATGGTCAAGAGAAAGGATTCCAG
AGGCTAG (SEQ ID NO. 414)
Cluster p Block V
MSIGLLCCVAFSLLWASPVNAGVTQTPKFQVLKTGQSMTLQCAQDMNHNSMYWYRQDPGMGLRLIYYSASEGTTDKGEVP
NGYNVSRLNKREFSLRLESAAPSQTSVYF* (SEQ ID NO. 415)
Seq-ID p.1b CASSGYSGGGA-DTQYF (SEQ ID NO. 380)
Seq-ID p.2b CASSGYSGGGA-DTQYF (SEQ ID NO. 381)
Seq-ID p.3b CASSD-SGGSA-DTQYF (SEQ ID NO. 382)
Seq-ID p.4b CASSGLAGGP--DTQYF (SEQ ID NO. 383)
Seq-ID p.5b CASSELGGGGT-DTQYF (SEQ ID NO. 384)
Seq-ID p.6b CASST--GGGT-DTQYF (SEQ ID NO. 385)
Seq-ID p.7b CASSERA-GGT-DTQYF (SEQ ID NO. 386)
Seq-ID p.8b CASSDSAGG-E-DTQYF (SEQ ID NO. 387)
Seq-ID p.9b CASSGGTE--T-DTQYF (SEQ ID NO. 388)
Seq-ID p.10b CASSGPTSGGPGDTQYF (SEQ ID NO. 389)
Seq-ID p.11b CASSQTSGGGA-DTQYF (SEQ ID NO. 390)
Seq-ID p.12b CASSELA-GGP-DTQYF (SEQ ID NO. 505)
Seq-ID p.13b CASSELA-GGP-ETQYF (SEQ ID NO. 506)
Cluster p Block J/C
GPGTRLTVLEDLKNVFPPKVAVFEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRADCGFTSESYQQGVLSATILYEILLGKATLY
AVLVSALVLMAMVKRKDSRG (SEQ ID NO. 416)
Alpha-chain, TRA: TRAV1-2, TRAJ33, TRAC*01
Cluster p Block V
ATGTGGGGAGTTTTCCTTCTTTATGTTTCCATGAAGATGGGAGGCACTACAGGACAAAACATTGACCAGCCCACTGAGATG
ACAGCTACGGAAGGTGCCATTGTCCAGATCAACTGCACGTACCAGACATCTGGGTTCAACGGGCTGTTCTGGTACCAGCA
ACATGCTGGCGAAGCACCCACATTTCTGTCTTACAATGTTCTGGATGGTTTGGAGGAGAAAGGTCGTTTTTCTTCATTCCTT
AGTCGGTCTAAAGGGTACAGTTACCTCCTTTTGAAGGAGCTCCAGATGAAAGACTCTGCCTCTTACCTC (SEQ ID NO.
417)
Seq-ID p.1a TGTGCTGTGATGGATAGCAACTATCAGTTAATCTGG (SEQ ID NO. 418)
Seq-ID p.2a TGTGCTGTTATGGATAGCAACTATCAGTTAATCTGG (SEQ ID NO. 419)
Seq-ID p.3a TGTGCTGTGAGAGATAGCAACTATCAGTTAATCTGG (SEQ ID NO. 420)
Seq-ID p.4a TGTGCTGTGAGGGATAGCAACTATCAGTTAATCTGG (SEQ ID NO. 421)
Seq-ID p.5a TGTGCCGTGAGGAGGGATAGCAACTATCAGTTAATCTGG (SEQ ID NO. 422)
Seq-ID p.6a TGTGCTGTCATGGATAGCAACTATCAGTTAATCTGG (SEQ ID NO. 423)
Seq-ID p.7a TGTGCCTCTTCCGATAGCAACTATCAGTTAATCTGG (SEQ ID NO. 424)
Seq-ID p.8a TGTGCTGTGAGAGATAGCAACTATCAGTTAATCTGG (SEQ ID NO. 425)
Seq-ID p.9a TGTGCTGTGAGAGATAGCAACTATCAGTTAATCTGG (SEQ ID NO. 426)
Seq-ID p.10a TGTGCTGTCCTGGATAGCAACTATCAGTTAATCTGG (SEQ ID NO. 427)
Seq-ID p.11a TGTGCTGTGAGGGATAGCAACTATCAGTTAATCTGG (SEQ ID NO. 428)
Seq-ID p.12a TGTGCTGTGAGAGATAGCAACTATCAGTTAATCTGG (SEQ ID NO. 512)
Seq-ID p.13a TGTGCTGTGAGAGATAGCAACTATCAGTTAATCTGG (SEQ ID NO. 513)
Cluster p Block J/C
GGCGCTGGGACCAAGCTAATTATAAAGCCAGATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTGATGTGTATATCA
CAGACAAAACTGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCTGTCAGTGATTGGGTTCCGAATCCTCCTCCTG
AAAGTGGCCGGGTTTAATCTGCTCATGACGCTGCGGCTGTGGTCCAGCTGA (SEQ ID NO. 429)
Cluster p Block V
MWGVFLLYVSMKMGGTTGQNIDQPTEMTATEGAIVQINCTYQTSGFNGLFWYQQHAGEAPTFLSYNVLDGLEEKGRFSSFLS
RSKGYSYLLLKELQMKDSASYL (SEQ ID NO. 430)
Seq-ID p.1a CAVM-DSNYQLIW (SEQ ID NO. 391)
Seq-ID p.2a CAVM-DSNYQLIW (SEQ ID NO. 392)
Seq-ID p.3a CAVR-DSNYQLIW (SEQ ID NO. 393)
Seq-ID p.4a CAVR-DSNYQLIW (SEQ ID NO. 394)
Seq-ID p.5a CAVRRDSNYQLIW (SEQ ID NO. 395)
Seq-ID p.6a CAVM-DSNYQLIW (SEQ ID NO. 396)
Seq-ID p.7a CASS-DSNYQLIW (SEQ ID NO. 397)
Seq-ID p.8a CAVR-DSNYQLIW (SEQ ID NO. 398)
Seq-ID p.9a CAVR-DSNYQLIW (SEQ ID NO. 399)
Seq-ID p.10a CAVL-DSNYQLIW (SEQ ID NO. 400)
Seq-ID p.11a CAVR-DSNYQLIW (SEQ ID NO. 401)
Seq-ID p.12a CAVR-DSNYQLIW (SEQ ID NO. 507)
Seq-ID p.13a CAVR-DSNYQLIW (SEQ ID NO. 508)
Cluster p Block J/C
GAGTKLIIKPDIQNPDPAVYQLRDSKSSDKSVCLFTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILLLKVAGFNLLMTLRLWSS* (SEQ ID NO. 431)

1. An isolated TCR polypeptide, wherein the TCR polypeptide comprises a CDR3 alpha sequence and a CDR3 beta sequence, wherein the CDR3 alpha sequence and the CDR3 beta sequence are identical to the sequences given below, or with one or two amino acid substitutions per CDR3 sequence,
wherein
a. for group a, the CDR3 alpha sequence is selected from the sequences SEQ ID NO 007-009 or SEQ ID NO 436-439, and the CDR3 beta sequence is selected from the sequences SEQ ID NO 001-006 or SEQ ID NO 432-435, or
b. for group b, the CDR3 alpha sequence is selected from the sequences SEQ ID NO 031-032, and the CDR3 beta sequence is selected from the sequences SEQ ID NO 027-030, or
c. for group c, the CDR3 alpha sequence is selected from the sequences SEQ ID NO 052-054, and the CDR3 beta sequence is selected from the sequences SEQ ID NO 048-051, or
d. for group d, the CDR3 alpha sequence is selected from the sequence SEQ ID NO 073, and the CDR3 beta sequence is selected from the sequences SEQ ID NO 070-072, or
e. for group e, the CDR3 alpha sequence is selected from the sequence SEQ ID NO 100 or SEQ ID NO 455-459, and the CDR3 beta sequence is selected from the sequences SEQ ID NO 086-099 or SEQ ID NO 450-454, or
f. for group f, the CDR3 alpha sequence is selected from the sequences SEQ ID NO 131-132 or SEQ ID NO 472-473, and the CDR3 beta sequence is selected from the sequences SEQ ID NO 124-130 or SEQ ID NO 470-471, or
g. for group g, the CDR3 alpha sequence is selected from the sequence SEQ ID NO 156, and the CDR3 beta sequence is selected from the sequences SEQ ID NO 150-155, or
h. for group h, the CDR3 alpha sequence is selected from the sequences SEQ ID NO 175-176, and the CDR3 beta sequence is selected from the sequences SEQ ID NO 172-174, or
i. for group i, the CDR3 alpha sequence is selected from the sequences SEQ ID NO 193-194, and the CDR3 beta sequence is selected from the sequences SEQ ID NO 190-192, or
j. for group j, the CDR3 alpha sequence is selected from the sequences SEQ ID NO 212-213 or SEQ ID NO 513, and the CDR3 beta sequence is selected from the sequences SEQ ID NO 208-211 or SEQ ID NO 478, or
k. for group k, the CDR3 alpha sequence is selected from the sequences SEQ ID NO 232-235, and the CDR3 beta sequence is selected from the sequences SEQ ID NO 228-231, or
l. for group l, the CDR3 alpha sequence is selected from the sequences SEQ ID NO 261-269 or SEQ ID NO 484-486, and the CDR3 beta sequence is selected from the sequences SEQ ID NO 252-260 or SEQ ID NO 481-483, or
m. for group m, the CDR3 alpha sequence is selected from the sequences SEQ ID NO 302-307 or SEQ ID NO 496-498, and the CDR3 beta sequence is selected from the sequences SEQ ID NO 296-301 or SEQ ID NO 493-495, or
n. for group n, the CDR3 alpha sequence is selected from the sequences SEQ ID NO 334-339, and the CDR3 beta sequence is selected from the sequences SEQ ID NO 328-333, or
o. for group o, the CDR3 alpha sequence is selected from the sequences SEQ ID NO 363-365, and the CDR3 beta sequence is selected from the sequences SEQ ID NO 360-362, or
p. for group p, the CDR3 alpha sequence is selected from the sequences SEQ ID NO 391-401 or SEQ ID NO 507-508, and the CDR3 beta sequence is selected from the sequences SEQ ID NO 380-390 or SEQ ID NO 505-506,
particularly wherein the CRD3 alpha sequence and the CDR3 beta sequence are identified in the same row of tables 1-16,
particularly wherein said substitutions are selected according to the substitution rules given below,
wherein the substitution rules are:
glycine (G) and alanine (A) are interchangeable; valine (V), leucine (L), and isoleucine (I) are interchangeable, A and V are interchangeable;
tryptophan (W) and phenylalanine (F) are interchangeable, tyrosine (Y) and F are interchangeable;
serine (S) and threonine (T) are interchangeable;
aspartic acid (D) and glutamic acid (E) are interchangeable
asparagine (N) and glutamine (Q) are interchangeable; N and S are interchangeable; N and D are interchangeable; E and Q are interchangeable;
methionine (M) and Q are interchangeable;
cysteine (C), A and S are interchangeable;
proline (P), G and A are interchangeable;
arginine (R) and lysine (K) are interchangeable.

2. The isolated TCR polypeptide according to claim 1, wherein the CDR3 sequences are selected from groups a, b, c, d, e, f, g, h, i, j, and k.

3. The isolated TCR polypeptide according to claim 1, wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences,
wherein
a. for group a, the V alpha sequence is SEQ ID NO 025, the JC alpha sequence is SEQ ID NO 026, the V beta sequence is SEQ ID NO 018, and the JC beta sequence is SEQ ID NO 019, or
b. for group b, the V alpha sequence is SEQ ID NO 046, the JC alpha sequence is SEQ ID NO 047, the V beta sequence is SEQ ID NO 040, and the JC beta sequence is SEQ ID NO 041, or
c. for group c, the V alpha sequence is SEQ ID NO 068, the JC alpha sequence is SEQ ID NO 069, the V beta sequence is SEQ ID NO 061, and the JC beta sequence is SEQ ID NO 062, or
d. for group d, the V alpha sequence is SEQ ID NO 084, the JC alpha sequence is SEQ ID NO 085, the V beta sequence is SEQ ID NO 079, and the JC beta sequence is SEQ ID NO 080, or
e. for group e, the V alpha sequence is SEQ ID NO 122, the JC alpha sequence is SEQ ID NO 0123, the V beta sequence is SEQ ID NO 117, and the JC beta sequence is SEQ ID NO 118, or
f. for group f, the V alpha sequence is SEQ ID NO 148, the JC alpha sequence is SEQ ID NO 149, the V beta sequence is SEQ ID NO 142, and the JC beta sequence is SEQ ID NO 143, or
g. for group g, the V alpha sequence is SEQ ID NO 170, the JC alpha sequence is SEQ ID NO 171, the V beta sequence is SEQ ID NO 165, and the JC beta sequence is SEQ ID NO 166, or
h. for group h, the V alpha sequence is SEQ ID NO 188, the JC alpha sequence is SEQ ID NO 189, the V beta sequence is SEQ ID NO 182, and the JC beta sequence is SEQ ID NO 183, or
i. for group i, the V alpha sequence is SEQ ID NO 206, the JC alpha sequence is SEQ ID NO 207, the V beta sequence is SEQ ID NO 200, and the JC beta sequence is SEQ ID NO 201, or
j. for group j, the V alpha sequence is SEQ ID NO 226, the JC alpha sequence is SEQ ID NO 227, the V beta sequence is SEQ ID NO 220, and the JC beta sequence is SEQ ID NO 221, or
k. for group k, the V alpha sequence is SEQ ID NO 250, the JC alpha sequence is SEQ ID NO 251, the V beta sequence is SEQ ID NO 242, and the JC beta sequence is SEQ ID NO 243, or
l. for group l, the V alpha sequence is SEQ ID NO 294, the JC alpha sequence is SEQ ID NO 295, the V beta sequence is SEQ ID NO 281, and the JC beta sequence is SEQ ID NO 282, or
m. for group m, the V alpha sequence is SEQ ID NO 326, the JC alpha sequence is SEQ ID NO 327, the V beta sequence is SEQ ID NO 316, and the JC beta sequence is SEQ ID NO 317, or
n. for group n, the V alpha sequence is SEQ ID NO 358, the JC alpha sequence is SEQ ID NO 359, the V beta sequence is SEQ ID NO 348, and the JC beta sequence is SEQ ID NO 349, or
o. for group o, the V alpha sequence is SEQ ID NO 378, the JC alpha sequence is SEQ ID NO 379, the V beta sequence is SEQ ID NO 371, and the JC beta sequence is SEQ ID NO 372, or
p. for group p, the V alpha sequence is SEQ ID NO 430, the JC alpha sequence is SEQ ID NO 431, the V beta sequence is SEQ ID NO 415, and the JC beta sequence is SEQ ID NO 416.

4. An isolated nucleic acid sequence encoding the TCR polypeptide according to claim 1.

5. An isolated autologous T cell comprising a TCR polypeptide according to claim 1.

6. The isolated autologous T cell according to claim 5, wherein the isolated autologous T cell is a recombinant T cell.

7. A method for treatment of cancer comprising administering to a patient in need thereof an agent selected from the TCR polypeptide according to claim 1, a nucleic acid sequence encoding the TCR polypeptide, an isolated autologous T cell comprising the TCR polypeptide, and an isolated autologous T cell comprising the nucleic acid encoding the TCR polypeptide.

8. The method according to claim 7, wherein the patient is characterized by the following HLA-type:
a. HLA-A*02:01 for group a; or
b. HLA-B*08:01 and/or HLA-C*07:01 for group b; or
c. HLA-A*02:01 for group c; or
d. HLA-A*02:01 for group d; or
e. HLA-B*15:01 for group e; or
f. HLA-A*02:01 for group f; or
g. HLA-B*08:01 for group g; or
h. HLA-B*07:02 for group h; or
i. HLA-A*01:01 and/or HLA-B*08:01 and/or HLA-C*07:01 for group i; or
j. HLA-A*02:01 for group j; or
k. HLA-A*02:01 for group k.

9. The method according to claim 7, wherein said cancer is a solid tumor, particularly wherein said cancer is selected from lung cancer, pancreatic cancer, colon cancer, and breast cancer.

10. The method according to claim 7, wherein said cancer is selected from the group of Bladder Urothelial Carcinoma, Breast invasive carcinoma, Cervical squamous cell carcinoma and endocervical adenocarcinoma, Cholangiocarcinoma, Colon adenocarcinoma, Lymphoid Neoplasm Diffuse Large B-cell Lymphoma, Esophageal carcinoma, Glioblastoma multiforme, Head and Neck squamous cell carcinoma, Kidney Chromophobe, Kidney renal papillary cell carcinoma, Acute Myeloid Leukemia, Brain Lower Grade Glioma, Lung adenocarcinoma, Lung squamous cell carcinoma, Mesothelioma, Ovarian serous cystadenocarcinoma, Pancreatic adenocarcinoma, Rectum adenocarcinoma, Sarcoma, Skin Cutaneous Melanoma, Stomach adenocarcinoma, Testicular Germ Cell Tumors, Thyroid carcinoma, Uterine Corpus Endometrial Carcinoma, Uterine Carcinosarcoma.

11. The method according to claim 7, wherein said cancer is characterized by a mutation in a gene selected from the group of
a. KRAS, EGFR, and/or TP53 for group a, particularly wherein the mutation of KRAS is a KRAS G12 mutation; or
b. KRAS, FGFR, and/or TP53 for group b, particularly wherein the mutation of KRAS is a KRAS Q61 mutation; or
c. KRAS, and/or EGFR for group c, particularly wherein the mutation of KRAS is a KRAS G12 or a KRAS Q61 mutation; or
d. KRAS and/or TP53 for group d, particularly wherein the mutation of KRAS is a KRAS G12 mutation; or
e. KRAS, EGFR and/or BRAF for group e, particularly wherein the mutation of KRAS is a KRAS G12 or a KRAS Q61 mutation; or
f. KRAS, EGFR and/or BRAF for group f, particularly wherein the mutation of KRAS is a KRAS G12 or a KRAS Q61 mutation; or
g. TP53 for group g; or
h. KRAS, EGFR, BRAF and/or TP53 for group h, particularly wherein the mutation of KRAS is a KRAS G12 or a KRAS Q61 mutation; or
i. EGFR, FGFR, and/or TP53 for group i; or
j. KRAS, EGFR and/or TP53, particularly wherein the mutation of KRAS is a KRAS G12 mutation; or
k. KRAS, EGFR, BRAF and/or TP53 for group k, particularly wherein the mutation of KRAS is a KRAS G12 or a KRAS Q61 mutation; or
n. KRAS, EGFR, BRAF and/or TP53 for group n, particularly wherein the mutation of KRAS is a KRAS G12 or a KRAS Q61 mutation; or
o. KRAS, EGFR, BRAF and/or TP53 for group o, particularly wherein the mutation of KRAS is a KRAS G12 or a KRAS Q61 mutation.

12. The method according to claim 7, wherein said cancer is characterized by a mutation in a gene selected from the group of
a. KRAS for group a, particularly wherein the mutation of KRAS is a KRAS G12 mutation; or
b. TP53 for group b; or
d. KRAS for group d, particularly wherein the mutation of KRAS is a KRAS G12 mutation; or
e. KRAS and/or EGFR for group e, particularly wherein the mutation of KRAS is a KRAS G12 mutation; or
f. KRAS and/or EGFR for group f, particularly wherein the mutation of KRAS is a KRAS G12 or a KRAS Q61 mutation; or
i. TP53 for group i; or
j. KRAS and/or EGFR, particularly wherein the mutation of KRAS is a KRAS G12 mutation; or
k. EGFR and/or TP53 for group k; or
n. KRAS, EGFR, and/or TP53 for group n, particularly wherein the mutation of KRAS is a KRAS G12 mutation; or
o. KRAS, EGFR and/or TP53 for group o, particularly wherein the mutation of KRAS is a KRAS G12 mutation.

13. A method for treatment of cancer comprising administering to a patient in need thereof an agent selected from
a TCR polypeptide,
an isolated nucleic acid sequence encoding the TCR polypeptide, or
an isolated autologous T cell comprising the TCR polypeptide and/or the nucleic acid sequence;
wherein the TCR polypeptide comprises a CDR3 alpha sequence and a CDR3 beta sequence, wherein the CDR3 alpha sequence and the CDR3 beta sequence are identical to the sequences given below, or with one amino acid substitution per CDR3 sequence, wherein the CDR3 alpha sequence is selected from the sequences SEQ ID NO 007-009 or SEQ ID NO 436-439, and the CDR3 beta sequence is selected from the sequences SEQ ID NO 001-006 or SEQ ID NO 432-435,
wherein the TCR polypeptide additionally comprises a variable (V) alpha sequence, a joining-constant (JC) alpha sequence, a V beta sequence, and a JC beta sequence or a sequence with ≥80%, ≥85%, ≥90%, ≥92%, ≥94%, ≥96%, ≥98%, or ≥99% sequence identity to said sequences, wherein the V alpha sequence is SEQ ID NO 025, the JC alpha sequence is SEQ ID NO 026, the V beta sequence is SEQ ID NO 018, and the JC beta sequence is SEQ ID NO 019,
wherein the agent is administered to a patient characterized by the HLA type HLA-A*02:01,
and wherein said cancer is characterized by a mutation in a gene of the EGFR-Raf-Ras pathway.

14. The method according to claim 13, wherein said mutation in a gene of the EGFR-Raf-Ras pathway is a mutation in KRAS and/or EGFR.

15. The method according to claim 13, wherein said mutation in a gene of the EGFR-Raf-Ras pathway is a KRAS G12 mutation.

16. An isolated autologous T cell comprising a nucleic acid sequence according to claim 4.


SEQUENCE LISTING (513 sequences)

SEQ ID NO: 1 - protein, 14 aa, Homo sapiens
CASSNDGMNTEAFF

SEQ ID NO: 2 - protein, 14 aa, Homo sapiens
CASSADGMNTEAFF

SEQ ID NO: 3 - protein, 14 aa, Homo sapiens
CASSEDGMNTEAFF

SEQ ID NO: 4 - protein, 14 aa, Homo sapiens
CASSDDGMNTEAFF

SEQ ID NO: 5 - protein, 14 aa, Homo sapiens
CASSTDGMNTEAFF

SEQ ID NO: 6 - protein, 14 aa, Homo sapiens
CASSEDGMNTEAFF

SEQ ID NO: 7 - protein, 13 aa, Homo sapiens
CAVLMDSNYQLIW

SEQ ID NO: 8 - protein, 13 aa, Homo sapiens
CAVLMDSNYQLIW

SEQ ID NO: 9 - protein, 13 aa, Homo sapiens
CAALMDSNYQLIW

SEQ ID NO: 10 - DNA, 327 bp, Homo sapiens
  source 1..327: mol_type = genomic DNA
atgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttc

SEQ ID NO: 11 - DNA, 42 bp, Homo sapiens
  source 1..42: mol_type = genomic DNA
tgcgccagctccaatgacgggatgaacactgaagctttcttt

SEQ ID NO: 12 - DNA, 42 bp, Homo sapiens
  source 1..42: mol_type = genomic DNA
tgcgccagcagcgcggacgggatgaacactgaagctttcttt

SEQ ID NO: 13 - DNA, 42 bp, Homo sapiens
  source 1..42: mol_type = genomic DNA
tgcgccagcagtgaggatggcatgaacactgaagctttcttt

SEQ ID NO: 14 - DNA, 42 bp, Homo sapiens
  source 1..42: mol_type = genomic DNA
tgcgccagcagtgacgacggcatgaacactgaagctttcttt

SEQ ID NO: 15 - DNA, 42 bp, Homo sapiens
  source 1..42: mol_type = genomic DNA
tgcgccagcagtaccgacgggatgaacactgaagctttcttt

SEQ ID NO: 16 - DNA, 42 bp, Homo sapiens; identical to SEQ ID NO: 13
  source 1..42: mol_type = genomic DNA

SEQ ID NO: 17 - DNA, 561 bp, Homo sapiens
  source 1..561: mol_type = genomic DNA
ggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 18 - protein, 109 aa, Homo sapiens
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYF

SEQ ID NO: 19 - protein, 186 aa, Homo sapiens
GQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 20 - DNA, 324 bp, Homo sapiens
  source 1..324: mol_type = genomic DNA
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctc

SEQ ID NO: 21 - DNA, 39 bp, Homo sapiens
  source 1..39: mol_type = genomic DNA
tgtgctgtgctcatggatagcaactatcagttaatctgg

SEQ ID NO: 22 - DNA, 39 bp, Homo sapiens
  source 1..39: mol_type = genomic DNA
tgtgctgtcctcatggatagcaactatcagttaatctgg

SEQ ID NO: 23 - DNA, 39 bp, Homo sapiens
  source 1..39: mol_type = genomic DNA
tgtgcggccttaatggatagcaactatcagttaatctgg

SEQ ID NO: 24 - DNA, 456 bp, Homo sapiens
  source 1..456: mol_type = genomic DNA
ggcgctgggaccaagctaattataaagccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 25 - protein, 108 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYL

SEQ ID NO: 26 - protein, 151 aa, Homo sapiens
GAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 27 - protein, 13 aa, Homo sapiens
CASSLGPNYEQYV

SEQ ID NO: 28 - protein, 13 aa, Homo sapiens
CASSSGPNYEQYF

SEQ ID NO: 29 - protein, 13 aa, Homo sapiens
CASSSQGPYEQYF

SEQ ID NO: 30 - protein, 13 aa, Homo sapiens
CASSIGPNYEQYV

SEQ ID NO: 31 - protein, 13 aa, Homo sapiens
CAASSNQGGKLIF

SEQ ID NO: 32 - protein, 13 aa, Homo sapiens
CAAFYNQGGKLIF

SEQ ID NO: 33 - DNA, 330 bp, Homo sapiens
  source 1..330: mol_type = genomic DNA
atgggcaccagtctcctatgctgggtggtcctgggtttcctagggacagatcacacaggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgc

SEQ ID NO: 34 - DNA, 39 bp, Homo sapiens
  source 1..39: mol_type = genomic DNA
tgtgccagcagcttaggcccgaattacgagcagtacgtc

SEQ ID NO: 35 - DNA, 39 bp, Homo sapiens
  source 1..39: mol_type = genomic DNA
tgtgccagcagctcgggaccaaactacgagcagtacttc

SEQ ID NO: 36 - DNA, 39 bp, Homo sapiens
  source 1..39: mol_type = genomic DNA
tgtgccagcagctcccaagggccctacgagcagtacttc

SEQ ID NO: 37 - DNA, 39 bp, Homo sapiens; identical to SEQ ID NO: 36
  source 1..39: mol_type = genomic DNA

SEQ ID NO: 38 - DNA, 39 bp, Homo sapiens
  source 1..39: mol_type = genomic DNA
tgtgccagcagcataggcccaaactacgagcagtacgtc

SEQ ID NO: 39 - DNA, 567 bp, Homo sapiens
  source 1..567: mol_type = genomic DNA
gggccgggcaccaggctcacggtcacagaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 40 - protein, 110 aa, Homo sapiens
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYR

SEQ ID NO: 41 - protein, 188 aa, Homo sapiens
GPGTRLTVTEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 42 - DNA, 324 bp, Homo sapiens
  source 1..324: mol_type = genomic DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttc

SEQ ID NO: 43 - DNA, 39 bp, Homo sapiens
  source 1..39: mol_type = genomic DNA
tgtgcagcaagtagtaaccagggaggaaagcttatcttc

SEQ ID NO: 44 - DNA, 39 bp, Homo sapiens
  source 1..39: mol_type = genomic DNA
tgtgcagccttttataaccagggaggaaagcttatcttc

SEQ ID NO: 45 - DNA, 456 bp, Homo sapiens
  source 1..456: mol_type = genomic DNA
ggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 46 - protein, 108 aa, Homo sapiens
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYF

SEQ ID NO: 47 - protein, 151 aa, Homo sapiens
GQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 48 - protein, 16 aa, Homo sapiens
CASSLEGQASSYEQYF

SEQ ID NO: 49 - protein, 16 aa, Homo sapiens
CASSLEGQAASYEQYF

SEQ ID NO: 50 - protein, 16 aa, Homo sapiens
CASSLEGQASSYEQYF

SEQ ID NO: 51 - protein, 16 aa, Homo sapiens
CASSLEGQASSYEQYF

SEQ ID NO: 52 - protein, 13 aa, Homo sapiens
CAGSGAGSYQLTF

SEQ ID NO: 53 - protein, 13 aa, Homo sapiens
CAGPGAGSYQLTF

SEQ ID NO: 54 - protein, 13 aa, Homo sapiens
CAGSGAGSYQLTF

SEQ ID NO: 55 - DNA, 327 bp, Homo sapiens
  source 1..327: mol_type = genomic DNA
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctt

SEQ ID NO: 56 - DNA, 48 bp, Homo sapiens
  source 1..48: mol_type = genomic DNA
tgcgccagcagcttggagggacaggcgagctcctacgagcagtacttc

SEQ ID NO: 57 - DNA, 48 bp, Homo sapiens
  source 1..48: mol_type = genomic DNA
tgcgccagcagcttggaaggacaggcagcctcctacgagcagtacttc

SEQ ID NO: 58 - DNA, 48 bp, Homo sapiens; identical to SEQ ID NO: 56
  source 1..48: mol_type = genomic DNA

SEQ ID NO: 59 - DNA, 48 bp, Homo sapiens
  source 1..48: mol_type = genomic DNA
tgcgccagcagcttggaggggcaggctagctcctacgagcagtacttc

SEQ ID NO: 60 - DNA, 567 bp, Homo sapiens; identical to SEQ ID NO: 39
  source 1..567: mol_type = genomic DNA

SEQ ID NO: 61 - protein, 109 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYL

SEQ ID NO: 62 - protein, 188 aa, Homo sapiens; identical to SEQ ID NO: 41

SEQ ID NO: 63 - DNA, 318 bp, Homo sapiens
  source 1..318: mol_type = genomic DNA
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttc

SEQ ID NO: 64 - DNA, 39 bp, Homo sapiens
  source 1..39: mol_type = genomic DNA
tgtgcaggatctggggctgggagttaccaactcactttc

SEQ ID NO: 65 - DNA, 39 bp, Homo sapiens
  source 1..39: mol_type = genomic DNA
tgtgcaggccctggggctgggagttaccaactcactttc

SEQ ID NO: 66 - DNA, 39 bp, Homo sapiens
  source 1..39: mol_type = genomic DNA
tgtgcggggtcgggggctgggagttaccaactcactttc

SEQ ID NO: 67 - DNA, 456 bp, Homo sapiens
  source 1..456: mol_type = genomic DNA
gggaaggggaccaaactctcggtcataccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 68 - protein, 106 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYF

SEQ ID NO: 69 - protein, 151 aa, Homo sapiens
GKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 70 - protein, 14 aa, Homo sapiens
CSVGAGGTNEKLFF

SEQ ID NO: 71 - protein, 14 aa, Homo sapiens
CSVGAGGTNEKLFF

SEQ ID NO: 72 - protein, 14 aa, Homo sapiens
CSVGTVATNEKLFF

SEQ ID NO: 73 - protein, 12 aa, Homo sapiens
CAESISAGKIIF

SEQ ID NO: 74 - DNA, 321 bp, Homo sapiens
  source 1..321: mol_type = genomic DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctc

SEQ ID NO: 75 - DNA, 42 bp, Homo sapiens
  source 1..42: mol_type = genomic DNA
tgcagcgttggagctggaggaactaatgaaaaactgtttttt

SEQ ID NO: 76 - DNA, 42 bp, Homo sapiens
  source 1..42: mol_type = genomic DNA
tgcagcgttggggcagggggcactaatgaaaaactgtttttt

SEQ ID NO: 77 - DNA, 42 bp, Homo sapiens
  source 1..42: mol_type = genomic DNA
tgcagcgtggggacggtggcaactaatgaaaaactgtttttt

SEQ ID NO: 78 - DNA, 561 bp, Homo sapiens
  source 1..561: mol_type = genomic DNA
ggcagtggaacccagctctctgtcttggaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 79 - protein, 107 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYL

SEQ ID NO: 80 - protein, 186 aa, Homo sapiens
GSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 81 - DNA, 327 bp, Homo sapiens
  source 1..327: mol_type = genomic DNA
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttc

SEQ ID NO: 82 - DNA, 36 bp, Homo sapiens
  source 1..36: mol_type = genomic DNA
tgtgcagagagtataagcgcgggcaagatcatcttt

SEQ ID NO: 83 - DNA, 456 bp, Homo sapiens
  source 1..456: mol_type = genomic DNA
ggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 84 - protein, 109 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYF

SEQ ID NO: 85 - protein, 151 aa, Homo sapiens
GKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 15 aa, Homo sapiens
CASSVTSGAYNEQFF

SEQ ID NO: 87 - protein, 15 aa, Homo sapiens
CASQGTSGAYNEQFF

SEQ ID NO: 88 - protein, 15 aa, Homo sapiens
CASSITSGNYNEQFF

SEQ ID NO: 89 - protein, 15 aa, Homo sapiens
CASSMTSGSYNEQFF

SEQ ID NO: 90 - protein, 15 aa, Homo sapiens
CASSPTSGSYNEQFF

SEQ ID NO: 91 - protein, 15 aa, Homo sapiens
CASSRTSGGYNEQFF

SEQ ID NO: 92 - protein, 15 aa, Homo sapiens
CASSVTSGAYNEQFF

SEQ ID NO: 93 - protein, 15 aa, Homo sapiens
CASSATSGNYNEQFF

SEQ ID NO: 94 - protein, 15 aa, Homo sapiens
CASSATSGSYNEQFF

SEQ ID NO: 95 - protein, 15 aa, Homo sapiens
CASSITSGDYNEQFF

SEQ ID NO: 96 - protein, 15 aa, Homo sapiens
CASSKTSGDYNEQFF

SEQ ID NO: 97 - protein, 15 aa, Homo sapiens
CASSPTSGQYNEQFF

SEQ ID NO: 98 - protein, 15 aa, Homo sapiens
CASSVTSGSYNEQFF

SEQ ID NO: 99 - protein, 15 aa, Homo sapiens
CASSLTSGDYNEQFF

SEQ ID NO: 100 - protein, 15 aa, Homo sapiens
CVVSAGREYGNKLVF

SEQ ID NO: 101 - DNA, 327 bp, Homo sapiens
  source 1..327: mol_type = genomic DNA
atgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctc

SEQ ID NO: 102 - DNA, 45 bp, Homo sapiens
  source 1..45: mol_type = genomic DNA
tgtgccagtagtgtcactagcggggcttacaatgagcagttcttc

SEQ ID NO: 103 - DNA, 45 bp, Homo sapiens
  source 1..45: mol_type = genomic DNA
tgtgccagtcaggggactagcggggcctacaatgagcagttcttc

SEQ ID NO: 104 - DNA, 45 bp, Homo sapiens
  source 1..45: mol_type = genomic DNA
tgtgccagtagtataactagcgggaactacaatgagcagttcttc

SEQ ID NO: 105 - DNA, 45 bp, Homo sapiens
  source 1..45: mol_type = genomic DNA
tgtgccagtagtatgactagcggttcctacaatgagcagttcttc

SEQ ID NO: 106 - DNA, 45 bp, Homo sapiens
  source 1..45: mol_type = genomic DNA
tgtgccagtagcccaactagcggatcctacaatgagcagttcttc

SEQ ID NO: 107 - DNA, 45 bp, Homo sapiens
  source 1..45: mol_type = genomic DNA
tgtgccagtagtcggactagcgggggctacaatgagcagttcttc

SEQ ID NO: 108 - DNA, 45 bp, Homo sapiens
  source 1..45: mol_type = genomic DNA
tgtgccagtagtgtaactagcggggcctacaatgagcagttcttc

SEQ ID NO: 109 - DNA, 45 bp, Homo sapiens
  source 1..45: mol_type = genomic DNA
tgtgccagtagtgcgactagcgggaactacaatgagcagttcttc

SEQ ID NO: 110 - DNA, 45 bp, Homo sapiens
  source 1..45: mol_type = genomic DNA
tgtgccagtagtgccactagcgggagctacaatgagcagttcttc

SEQ ID NO: 111 - DNA, 45 bp, Homo sapiens
  source 1..45: mol_type = genomic DNA
tgtgccagtagtataactagcggagattacaatgagcagttcttc

SEQ ID NO: 112 - DNA, 45 bp, Homo sapiens
  source 1..45: mol_type = genomic DNA
tgtgccagtagtaaaactagcggagactacaatgagcagttcttc

SEQ ID NO: 113 - DNA, 45 bp, Homo sapiens
  source 1..45: mol_type = genomic DNA
tgtgccagtagtcccactagcggccagtacaatgagcagttcttc

SEQ ID NO: 114 - DNA, 45 bp, Homo sapiens
  source 1..45: mol_type = genomic DNA
tgtgccagtagcgtgactagcggctcctacaatgagcagttcttc

SEQ ID NO: 115 - DNA, 45 bp, Homo sapiens
  source 1..45: mol_type = genomic DNA
tgtgccagtagtttgactagcggggactacaatgagcagttcttc

SEQ ID NO: 116 - DNA, 567 bp, Homo sapiens
  source 1..567: mol_type = genomic DNA
gggccagggacacggctcaccgtgctagaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 117 - protein, 109 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYL

SEQ ID NO: 118 - protein, 188 aa, Homo sapiens
GPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 119 - DNA, 330 bp, Homo sapiens
  source 1..330: mol_type = genomic DNA
atgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatc

SEQ ID NO: 120 - DNA, 45 bp, Homo sapiens
  source 1..45: mol_type = genomic DNA
tgtgtggtgagcgccgggagggaatatggaaacaaactggtcttt

SEQ ID NO: 121 - DNA, 456 bp, Homo sapiens
  source 1..456: mol_type = genomic DNA
ggcgcaggaaccattctgagagtcaagtcctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 122 - protein, 110 aa, Homo sapiens
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYI

SEQ ID NO: 123 - protein, 151 aa, Homo sapiens
GAGTILRVKSYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 124 - protein, 15 aa, Homo sapiens
CASSTTSGAYNEQFF

SEQ ID NO: 125 - protein, 15 aa, Homo sapiens
CASSTTSGDYNEQFF

SEQ ID NO: 126 - protein, 15 aa, Homo sapiens
CASSVTSGAYNEQFF

SEQ ID NO: 127 - protein, 15 aa, Homo sapiens
CASSPTSGQYNEQFF

SEQ ID NO: 128 - protein, 15 aa, Homo sapiens
CASSQTSGGYNEQFF

SEQ ID NO: 129 - protein, 15 aa, Homo sapiens
CASSVTSGAYNEQFF

SEQ ID NO: 130 - protein, 15 aa, Homo sapiens
CASSLTSGGYNEQFF

SEQ ID NO: 131 - protein, 19 aa, Homo sapiens
CAVNRGRDAGGTSYGKLTF

SEQ ID NO: 132 - protein, 19 aa, Homo sapiens
CAVKLGRDAGGTSYGKLTF

SEQ ID NO: 133 - DNA, 327 bp, Homo sapiens; identical to SEQ ID NO: 101
  source 1..327: mol_type = genomic DNA

SEQ ID NO: 134 - DNA, 45 bp, Homo sapiens
  source 1..45: mol_type = genomic DNA
tgtgccagtagtaccacctccggggcctacaatgagcagttcttc

SEQ ID NO: 135 - DNA, 45 bp, Homo sapiens
  source 1..45: mol_type = genomic DNA
tgtgccagtagtacgactagcggggactacaatgagcagttcttc

SEQ ID NO: 136 - DNA, 45 bp, Homo sapiens
  source 1..45: mol_type = genomic DNA
tgtgccagtagtgtaactagcggggcttacaatgagcagttcttc

SEQ ID NO: 137 - DNA, 45 bp, Homo sapiens
  source 1..45: mol_type = genomic DNA
tgtgccagtagcccgactagcggacaatacaatgagcagttcttc

SEQ ID NO: 138 - DNA, 45 bp, Homo sapiens
  source 1..45: mol_type = genomic DNA
tgtgccagtagccaaactagcgggggatacaatgagcagttcttc

SEQ ID NO: 139 - DNA, 45 bp, Homo sapiens
  source 1..45: mol_type = genomic DNA
tgtgccagtagtgtcactagcggggcctacaatgagcagttcttc

SEQ ID NO: 140 - DNA, 45 bp, Homo sapiens
  source 1..45: mol_type = genomic DNA
tgtgccagtagcctaactagcgggggatacaatgagcagttcttc

SEQ ID NO: 141 - DNA, 567 bp, Homo sapiens; identical to SEQ ID NO: 116
  source 1..567: mol_type = genomic DNA

SEQ ID NO: 142 - protein, 109 aa, Homo sapiens; identical to SEQ ID NO: 117

SEQ ID NO: 143 - protein, 188 aa, Homo sapiens; identical to SEQ ID NO: 118

SEQ ID NO: 144 - DNA, 327 bp, Homo sapiens
  source 1..327: mol_type = genomic DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctc

SEQ ID NO: 145 - DNA, 57 bp, Homo sapiens
  source 1..57: mol_type = genomic DNA
tgtgccgtgaataggggaagggatgctggtggtactagctatggaaagctgacattt

SEQ ID NO: 146 - DNA, 57 bp, Homo sapiens
  source 1..57: mol_type = genomic DNA
tgtgccgtgaaattagggcgggatgctggtggtactagctatggaaagctgacattt

SEQ ID NO: 147 - DNA, 456 bp, Homo sapiens
  source 1..456: mol_type = genomic DNA
ggacaagggaccatcttgactgtccatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 148 - protein, 109 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYL

SEQ ID NO: 149 - protein, 151 aa, Homo sapiens
GQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 150 - protein, 13 aa, Homo sapiens
CASSYDSSYEQYF

SEQ ID NO: 151 - protein, 13 aa, Homo sapiens
CASSYDSSYEQYV

SEQ ID NO: 152 - protein, 13 aa, Homo sapiens
CASSWDSSYEQYF

SEQ ID NO: 153 - protein, 13 aa, Homo sapiens
CASSIDSSYEQYF

SEQ ID NO: 154 - protein, 13 aa, Homo sapiens
CASSIDSSYEQYF

SEQ ID NO: 155 - protein, 13 aa, Homo sapiens
CASTVDSSYEQYF

SEQ ID NO: 156 - protein, 13 aa, Homo sapiens
CAGPYFGNEKLTF

SEQ ID NO: 157 - DNA, 327 bp, Homo sapiens
  source 1..327: mol_type = genomic DNA
atgagcctcgggctcctgtgctgtggggccttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttc

SEQ ID NO: 158 - DNA, 39 bp, Homo sapiens
  source 1..39: mol_type = genomic DNA
tgtgccagcagttacgacagctcctacgagcagtacttc

SEQ ID NO: 159 - DNA, 39 bp, Homo sapiens
  source 1..39: mol_type = genomic DNA
tgtgccagcagttacgacagctcctacgagcagtacgtc

SEQ ID NO: 160 - DNA, 39 bp, Homo sapiens
  source 1..39: mol_type = genomic DNA
tgtgccagcagctgggactcctcctacgagcagtacttc

SEQ ID NO: 161 - DNA, 39 bp, Homo sapiens
  source 1..39: mol_type = genomic DNA
tgtgccagctcgatagacagctcctacgagcagtacttc

SEQ ID NO: 162 - DNA, 39 bp, Homo sapiens
  source 1..39: mol_type = genomic DNA
tgtgccagcagtatagacagctcctacgagcagtacttc

SEQ ID NO: 163 - DNA, 39 bp, Homo sapiens
  source 1..39: mol_type = genomic DNA
tgtgccagcaccgtcgactcgtcctacgagcagtacttc

SEQ ID NO: 164 - DNA, 567 bp, Homo sapiens; identical to SEQ ID NO: 39
  source 1..567: mol_type = genomic DNA

SEQ ID NO: 165 - protein, 109 aa, Homo sapiens
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYF

SEQ ID NO: 166 - protein, 188 aa, Homo sapiens; identical to SEQ ID NO: 41

SEQ ID NO: 167 - DNA, 318 bp, Homo sapiens
  source 1..318: mol_type = genomic DNA
atgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttc

SEQ ID NO: 168 - DNA, 39 bp, Homo sapiens
  source 1..39: mol_type = genomic DNA
tgtgctggcccctactttggaaatgagaaattaaccttt

SEQ ID NO: 169 - DNA, 456 bp, Homo sapiens
  source 1..456: mol_type = genomic DNA
gggactggaacaagactcaccatcatacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 170 - protein, 106 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYF

SEQ ID NO: 171 - protein, 151 aa, Homo sapiens
GTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 172 - protein, 13 aa, Homo sapiens
CASSLAGDQPQHF

SEQ ID NO: 173 - protein, 13 aa, Homo sapiens
CASSLEGDRPQHF

SEQ ID NO: 174 - protein, 13 aa, Homo sapiens
CASSLEGDQPQHF

SEQ ID NO: 175 - protein, 11 aa, Homo sapiens
CAVRYGQNFVF

SEQ ID NO: 176 - protein, 11 aa, Homo sapiens
CAVRYGQNFVF

SEQ ID NO: 177 - DNA, 327 bp, Homo sapiens; identical to SEQ ID NO: 55
  source 1..327: mol_type = genomic DNA

SEQ ID NO: 178 - DNA, 39 bp, Homo sapiens
  source 1..39: mol_type = genomic DNA
tgcgccagcagcttggcaggggatcagccccagcatttt

SEQ ID NO: 179 - DNA, 39 bp, Homo sapiens
  source 1..39: mol_type = genomic DNA
tgcgccagcagcttggaaggggaccgaccccagcatttt

SEQ ID NO: 180 - DNA, 39 bp, Homo sapiens
  source 1..39: mol_type = genomic DNA
tgcgccagcagcttggagggggatcagccccagcatttt

SEQ ID NO: 181 - DNA, 561 bp, Homo sapiens
  source 1..561: mol_type = genomic DNA
ggtgatgggactcgactctccatcctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 182 - protein, 109 aa, Homo sapiens; identical to SEQ ID NO: 61

SEQ ID NO: 183 - protein, 186 aa, Homo sapiens
GDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 184 - DNA, 318 bp, Homo sapiens
  source 1..318: mol_type = genomic DNA
atgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttc

SEQ ID NO: 185 - DNA, 33 bp, Homo sapiens
  source 1..33: mol_type = genomic DNA
tgtgctgtccgctatggtcagaattttgtcttt

SEQ ID NO: 186 - DNA, 33 bp, Homo sapiens; identical to SEQ ID NO: 185
  source 1..33: mol_type = genomic DNA

SEQ ID NO: 187 - DNA, 456 bp, Homo sapiens
  source 1..456: mol_type = genomic DNA
ggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 188 - protein, 106 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYF

SEQ ID NO: 189 - protein, 151 aa, Homo sapiens
GPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 190 - protein, 15 aa, Homo sapiens
CASSSSGAGDQPQHF

SEQ ID NO: 191 - protein, 15 aa, Homo sapiens
CASSSGTGGNQPQHF

SEQ ID NO: 192 - protein, 14 aa, Homo sapiens
CASSSEGAGQPQHF

SEQ ID NO: 193 - protein, 13 aa, Homo sapiens
CAASETSYDKVIF

SEQ ID NO: 194 - protein, 13 aa, Homo sapiens
CAASSTSYDKVIF

SEQ ID NO: 195 - DNA, 375 bp, Homo sapiens
  source 1..375: mol_type = genomic DNA
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctcctccggggccggggatcagccccagcatttt

SEQ ID NO: 196 - DNA, 45 bp, Homo sapiens
  source 1..45: mol_type = genomic DNA
tgtgccagcagctcctccggggccggggatcagccccagcatttt

SEQ ID NO: 197 - DNA, 45 bp, Homo sapiens
  source 1..45: mol_type = genomic DNA
tgtgccagcagctctgggacagggggcaatcagccccagcatttt

SEQ ID NO: 198 - DNA, 42 bp, Homo sapiens
  source 1..42: mol_type = genomic DNA
tgtgccagcagctccgagggggccggtcagccccagcatttt

SEQ ID NO: 199 - DNA, 561 bp, Homo sapiens; identical to SEQ ID NO: 181
  source 1..561: mol_type = genomic DNA

SEQ ID NO: 200 - protein, 110 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYL

SEQ ID NO: 201 - protein, 186 aa, Homo sapiens; identical to SEQ ID NO: 183

SEQ ID NO: 202 - DNA, 324 bp, Homo sapiens
  source 1..324: mol_type = genomic DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttc

SEQ ID NO: 203 - DNA, 39 bp, Homo sapiens
  source 1..39: mol_type = genomic DNA
tgtgcagcaagtgaaacctcctacgacaaggtgatattt

SEQ ID NO: 204 - DNA, 39 bp, Homo sapiens
  source 1..39: mol_type = genomic DNA
tgtgcagcaagtagcacctcctacgacaaggtgatattt

SEQ ID NO: 205 - DNA, 456 bp, Homo sapiens
  source 1..456: mol_type = genomic DNA
gggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 206 - protein, 108 aa, Homo sapiens [sequence omitted]

SEQ ID NO: 207 - protein, 151 aa, Homo sapiens
GPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 208 - protein, 13 aa, Homo sapiens
CSARVGVGNTIYF

SEQ ID NO: 209 - protein, 13 aa, Homo sapiens
CSARVGVGNTIYF

SEQ ID NO: 210 - protein, 13 aa, Homo sapiens
CSARDQVGNTIYF

SEQ ID NO: 211 - protein, 13 aa, Homo sapiens
CSARAGVGNTIYF

SEQ ID NO: 212 - protein, 11 aa, Homo sapiens
CAEDNNARLMF

SEQ ID NO: 213 - protein, 11 aa, Homo sapiens
CAEDLNARLMF

SEQ ID NO: 214 - DNA, 321 bp, Homo sapiens
  source 1..321: mol_type = genomic DNA
atgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatc

SEQ ID NO: 215 - DNA, 39 bp, Homo sapiens
  source 1..39: mol_type = genomic DNA
tgcagtgctagagtaggggttggaaacaccatatatttt

SEQ ID NO: 216 - DNA, 39 bp, Homo sapiens
  source 1..39: mol_type = genomic DNA
tgcagtgctagagttggggttggaaacaccatatatttt

SEQ ID NO: 217 - DNA, 39 bp, Homo sapiens
  source 1..39: mol_type = genomic DNA
tgcagtgctagagaccaggttggaaacaccatatatttt

SEQ ID NO: 218 - DNA, 39 bp, Homo sapiens
  source 1..39: mol_type = genomic DNA
tgcagtgctagggcaggggtaggaaacaccatatatttt

SEQ ID NO: 219 - DNA, 561 bp, Homo sapiens
  source 1..561: mol_type = genomic DNA
ggagagggaagttggctcactgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 220 - protein, 107 aa, Homo sapiens
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYI

SEQ ID NO: 221 - protein, 186 aa, Homo sapiens
GEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 222 - DNA, 327 bp, Homo sapiens; identical to SEQ ID NO: 81
  source 1..327: mol_type = genomic DNA

SEQ ID NO: 223 - DNA, 33 bp, Homo sapiens
  source 1..33: mol_type = genomic DNA
tgtgcagaggataacaatgccagactcatgttt

SEQ ID NO: 224 - DNA, 33 bp, Homo sapiens
  source 1..33: mol_type = genomic DNA
tgtgcagaggatcttaatgccagactcatgttt

SEQ ID NO: 225 - DNA, 456 bp, Homo sapiens
  source 1..456: mol_type = genomic DNA
ggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 226 - protein, 109 aa, Homo sapiens; identical to SEQ ID NO: 84

SEQ ID NO: 227 - protein, 151 aa, Homo sapiens
GDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 228 - protein, 12 aa, Homo sapiens
CASSTGAYGYTF

SEQ ID NO: 229 - protein, 12 aa, Homo sapiens
CASSIGIYGYTF

SEQ ID NO: 230 - protein, 12 aa, Homo sapiens
CASSIGWHGYTF

SEQ ID NO: 231 - protein, 12 aa, Homo sapiens
CASSQGVYGYTF

SEQ ID NO: 232 - protein, 17 aa, Homo sapiens
CAFMTNAGGTSYGKLTF

SEQ ID NO: 233 - protein, 17 aa, Homo sapiens
CAYSPNAGGTSYGKLTF

SEQ ID NO: 234 - protein, 17 aa, Homo sapiens
CAFMLNAGGTSYGKLTF

SEQ ID NO: 235 - protein, 17 aa, Homo sapiens
CAYHLSAGGTSYGKLTF

SEQ ID NO: 236 - DNA, 327 bp, Homo sapiens; identical to SEQ ID NO: 101
  source 1..327: mol_type = genomic DNA

SEQ ID NO: 237 - DNA, 36 bp, Homo sapiens
  source 1..36: mol_type = genomic DNA
tgtgccagtagtacaggggcctatggctacaccttc

SEQ ID NO: 238 - DNA, 36 bp, Homo sapiens
  source 1..36: mol_type = genomic DNA
tgtgccagtagtatagggatctatggctacaccttc

SEQ ID NO: 239 - DNA, 36 bp, Homo sapiens
  source 1..36: mol_type = genomic DNA
tgtgccagtagtatcggttggcatggctacaccttc

SEQ ID NO: 240 - DNA, 36 bp, Homo sapiens
  source 1..36: mol_type = genomic DNA
tgtgccagtagccagggggtctatggctacaccttc

SEQ ID NO: 241 - DNA, 561 bp, Homo sapiens
  source 1..561: mol_type = genomic DNA
ggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 242 - protein, 109 aa, Homo sapiens; identical to SEQ ID NO: 117

SEQ ID NO: 243 - protein, 186 aa, Homo sapiens
GSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 244 - DNA, 333 bp, Homo sapiens
  source 1..333: mol_type = genomic DNA
atgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttc

SEQ ID NO: 245 - DNA, 51 bp, Homo sapiens
  source 1..51: mol_type = genomic DNA
tgtgctttcatgaccaatgctggtggtactagctatggaaagctgacattt

SEQ ID NO: 246 - DNA, 51 bp, Homo sapiens
  source 1..51: mol_type = genomic DNA
tgtgcttatagccccaatgctggtggtactagctatggaaagctgacattt

SEQ ID NO: 247 - DNA, 51 bp, Homo sapiens
  source 1..51: mol_type = genomic DNA
tgtgctttcatgcttaatgctggtggtactagctatggaaagctgacattt

SEQ ID NO: 248 - DNA, 51 bp, Homo sapiens
  source 1..51: mol_type = genomic DNA
tgtgcttatcacctcagtgctggtggtactagctatggaaagctgacattt

SEQ ID NO: 249 - DNA, 456 bp, Homo sapiens; identical to SEQ ID NO: 147
  source 1..456: mol_type = genomic DNA

SEQ ID NO: 250 - protein, 111 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYF

SEQ ID NO: 251 - protein, 151 aa, Homo sapiens; identical to SEQ ID NO: 149

SEQ ID NO: 252 - protein, 15 aa, Homo sapiens
CASSSDRGSTDTQYF

SEQ ID NO: 253 - protein, 15 aa, Homo sapiens
CASSERRGDTDTQYF

SEQ ID NO: 254 - protein, 15 aa, Homo sapiens
CASSPGTGDTDTQYF

SEQ ID NO: 255 - protein, 15 aa, Homo sapiens
CASSPGTGDTDTQYF

SEQ ID NO: 256 - protein, 15 aa, Homo sapiens
CASSERAGGTDTQYF

SEQ ID NO: 257 - protein, 13 aa, Homo sapiens
CASSEQGTDTQYF

SEQ ID NO: 258 - protein, 14 aa, Homo sapiens
CASSETSDTDTQYF

SEQ ID NO: 259 - protein, 14 aa, Homo sapiens
CASSTGGGTDTQYF

SEQ ID NO: 260 - protein, 15 aa, Homo sapiens
CASSDSRGGTDTQYF

SEQ ID NO: 261 - protein, 12 aa, Homo sapiens
CAVRDSNYQLIW

SEQ ID NO: 262 - protein, 12 aa, Homo sapiens
CASMDSNYQLIW

SEQ ID NO: 263 - protein, 12 aa, Homo sapiens
CAVMDSNYQLIW

SEQ ID NO: 264 - protein, 12 aa, Homo sapiens
CAVMDSNYQLIW

SEQ ID NO: 265 - protein, 12 aa, Homo sapiens
CASSDSNYQLIW

SEQ ID NO: 266 - protein, 12 aa, Homo sapiens
CSCMDSNYQLIW

SEQ ID NO: 267 - protein, 12 aa, Homo sapiens
CAVRDSNYQLIW

SEQ ID NO: 268 - protein, 12 aa, Homo sapiens
CAVMDSNYQLIW

SEQ ID NO: 269 - protein, 12 aa, Homo sapiens
CAVMDSNYQLIW

SEQ ID NO: 270 - DNA, 327 bp, Homo sapiens
  source 1..327: mol_type = genomic DNA
atgagaatcaggctcctgtgctgtgtggccttttctctcctgtgggcaggtccagtgattgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttc

SEQ ID NO: 271 - DNA, 45 bp, Homo sapiens
  source 1..45: mol_type = genomic DNA
tgtgccagcagcagcgacaggggaagcacagatacgcagtatttt

SEQ ID NO: 272 - DNA, 45 bp, Homo sapiens
  source 1..45: mol_type = genomic DNA
tgtgccagcagtgagaggcggggggacacagatacgcagtatttt

SEQ ID NO: 273 - DNA, 45 bp, Homo sapiens
  source 1..45: mol_type = genomic DNA
tgtgccagcagccccgggacaggggacacagatacgcagtatttt

SEQ ID NO: 274 - DNA, 45 bp, Homo sapiens; identical to SEQ ID NO: 273
  source 1..45: mol_type = genomic DNA

SEQ ID NO: 275 - DNA, 45 bp, Homo sapiens
  source 1..45: mol_type = genomic DNA
tgtgccagcagtgagagagcggggggcacagatacgcagtatttt

SEQ ID NO: 276 - DNA, 39 bp, Homo sapiens
  source 1..39: mol_type = genomic DNA
tgtgccagcagtgaacaaggcacagatacgcagtatttt

SEQ ID NO: 277 - DNA, 42 bp, Homo sapiens
  source 1..42: mol_type = genomic DNA
tgtgccagcagtgaaactagcgacacagatacgcagtatttt

SEQ ID NO: 278 - DNA, 42 bp, Homo sapiens
  source 1..42: mol_type = genomic DNA
tgtgccagcagtacggggggcggcacagatacgcagtatttt

SEQ ID NO: 279 - DNA, 45 bp, Homo sapiens
  source 1..45: mol_type = genomic DNA
tgtgccagcagtgactcgcgggggggcacagatacgcagtatttt

SEQ ID NO: 280 - DNA, 567 bp, Homo sapiens
  source 1..567: mol_type = genomic DNA
ggcccaggcacccggctgacagtgctcgaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 281 - protein, 109 aa, Homo sapiens
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYF

SEQ ID NO: 282 - protein, 188 aa, Homo sapiens; identical to SEQ ID NO: 118

SEQ ID NO: 283 - DNA, 312 bp, Homo sapiens
  source 1..312: mol_type = genomic DNA
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctc

SEQ ID NO: 284 - DNA, 36 bp, Homo sapiens
  source 1..36: mol_type = genomic DNA
tgtgctgtgagggatagcaactatcagttaatctgg

SEQ ID NO: 285 - DNA, 36 bp, Homo sapiens
  source 1..36: mol_type = genomic DNA
tgtgcttccatggatagcaactatcagttaatctgg

SEQ ID NO: 286 - DNA, 36 bp, Homo sapiens
  source 1..36: mol_type = genomic DNA
tgtgctgtgatggatagcaactatcagttaatctgg

SEQ ID NO: 287 - DNA, 36 bp, Homo sapiens; identical to SEQ ID NO: 286
  source 1..36: mol_type = genomic DNA

SEQ ID NO: 288 - DNA, 36 bp, Homo sapiens
  source 1..36: mol_type = genomic DNA
tgtgcctcttccgatagcaactatcagttaatctgg

SEQ ID NO: 289 - DNA, 36 bp, Homo sapiens
  source 1..36: mol_type = genomic DNA
tgctcgtgcatggatagcaactatcagttaatctgg

SEQ ID NO: 290 - DNA, 36 bp, Homo sapiens
  source 1..36: mol_type = genomic DNA
tgtgctgtgagagatagcaactatcagttaatctgg

SEQ ID NO: 291 - DNA, 36 bp, Homo sapiens
  source 1..36: mol_type = genomic DNA
tgtgctgtcatggatagcaactatcagttaatctgg

SEQ ID NO: 292 - DNA, 36 bp, Homo sapiens; identical to SEQ ID NO: 286
  source 1..36: mol_type = genomic DNA

SEQ ID NO: 293 - DNA, 456 bp, Homo sapiens; identical to SEQ ID NO: 24
  source 1..456: mol_type = genomic DNA

SEQ ID NO: 294 - protein, 104 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYL

SEQ ID NO: 295 - protein, 151 aa, Homo sapiens; identical to SEQ ID NO: 26

SEQ ID NO: 296 - protein, 13 aa, Homo sapiens
CASSDSTTDTQYF

SEQ ID NO: 297 - protein, 14 aa, Homo sapiens
CASSDSISTDTQYF

SEQ ID NO: 298 - protein, 15 aa, Homo sapiens
CASSDSAGGEDTQYF

SEQ ID NO: 299 - protein, 14 aa, Homo sapiens
CASSENQGADTQYF

SEQ ID NO: 300 - protein, 15 aa, Homo sapiens
CASSDSGGSADTQYF

SEQ ID NO: 301 - protein, 14 aa, Homo sapiens
CASSDGDSADTQYF

SEQ ID NO: 302 - protein, 12 aa, Homo sapiens
CAVMDSNYQLIW

SEQ ID NO: 303 - protein, 12 aa, Homo sapiens
CAVVDSNYQLIW

SEQ ID NO: 304 - protein, 12 aa, Homo sapiens
CAVRDSNYQLIW

SEQ ID NO: 305 - protein, 12 aa, Homo sapiens
CAPMNSNYQLIW

SEQ ID NO: 306 - protein, 12 aa, Homo sapiens
CAVRDSNYQLIW

SEQ ID NO: 307 - protein, 12 aa, Homo sapiens
CAVKDSNYQLIW

SEQ ID NO: 308 - DNA, 327 bp, Homo sapiens; identical to SEQ ID NO: 270
  source 1..327: mol_type = genomic DNA

SEQ ID NO: 309 - DNA, 39 bp, Homo sapiens
  source 1..39: mol_type = genomic DNA
tgtgccagcagtgactcgaccacagatacgcagtatttt

SEQ ID NO: 310 - DNA, 42 bp, Homo sapiens
  source 1..42: mol_type = genomic DNA
tgtgccagcagtgactcgatcagcacagatacgcagtatttt

SEQ ID NO: 311 - DNA, 45 bp, Homo sapiens
  source 1..45: mol_type = genomic DNA
tgtgccagcagtgactccgcggggggcgaagatacgcagtatttt

SEQ ID NO: 312 - DNA, 42 bp, Homo sapiens
  source 1..42: mol_type = genomic DNA
tgtgccagcagtgaaaatcagggggcagatacgcagtatttt

SEQ ID NO: 313 - DNA, 45 bp, Homo sapiens
  source 1..45: mol_type = genomic DNA
tgtgccagcagtgactccggagggagcgcagatacgcagtatttt

SEQ ID NO: 314 - DNA, 42 bp, Homo sapiens
  source 1..42: mol_type = genomic DNA
tgtgccagcagtgacggggattcggcagatacgcagtatttt

SEQ ID NO: 315 - DNA, 567 bp, Homo sapiens; identical to SEQ ID NO: 280
  source 1..567: mol_type = genomic DNA

SEQ ID NO: 316 - protein, 109 aa, Homo sapiens; identical to SEQ ID NO: 281

SEQ ID NO: 317 - protein, 188 aa, Homo sapiens; identical to SEQ ID NO: 118

SEQ ID NO: 318 - DNA, 312 bp, Homo sapiens; identical to SEQ ID NO: 283
  source 1..312: mol_type = genomic DNA

SEQ ID NO: 319 - DNA, 36 bp, Homo sapiens; identical to SEQ ID NO: 286
  source 1..36: mol_type = genomic DNA

SEQ ID NO: 320 - DNA, 36 bp, Homo sapiens
  source 1..36: mol_type = genomic DNA
tgtgctgtggtggatagcaactatcagttaatctgg

SEQ ID NO: 321 - DNA, 36 bp, Homo sapiens; identical to SEQ ID NO: 290
  source 1..36: mol_type = genomic DNA

SEQ ID NO: 322 - DNA, 36 bp, Homo sapiens
  source 1..36: mol_type = genomic DNA
tgtgcccccatgaatagcaactatcagttaatctgg

SEQ ID NO: 323 - DNA, 36 bp, Homo sapiens; identical to SEQ ID NO: 290
  source 1..36: mol_type = genomic DNA

SEQ ID NO: 324 - DNA, 36 bp, Homo sapiens
  source 1..36: mol_type = genomic DNA
tgtgctgtgaaggatagcaactatcagttaatctgg

SEQ ID NO: 325 - DNA, 456 bp, Homo sapiens; identical to SEQ ID NO: 24
  source 1..456: mol_type = genomic DNA

SEQ ID NO: 326 - protein, 104 aa, Homo sapiens; identical to SEQ ID NO: 294

SEQ ID NO: 327 - protein, 151 aa, Homo sapiens; identical to SEQ ID NO: 26

SEQ ID NO: 328 - protein, 16 aa, Homo sapiens
CASSEVAGQGTGELFF

SEQ ID NO: 329 - protein, 13 aa, Homo sapiens
CASSAGDTGELFF

SEQ ID NO: 330 - protein, 16 aa, Homo sapiens
CASSEAGTGDTGELFF

SEQ ID NO: 331 - protein, 14 aa, Homo sapiens
CASSVGGGTGELFF

SEQ ID NO: 332 - protein, 15 aa, Homo sapiens
CASSDGSGGTGELFF

SEQ ID NO: 333 - protein, 15 aa, Homo sapiens
CASSDDAGGTGELFF

SEQ ID NO: 334 - protein, 12 aa, Homo sapiens
CAVRDSNYQLIW

SEQ ID NO: 335 - protein, 12 aa, Homo sapiens
CATADSNYQLIW

SEQ ID NO: 336 - protein, 12 aa, Homo sapiens
CAVLDSNYQLIW

SEQ ID NO: 337 - protein, 12 aa, Homo sapiens
CAVRDSNYQLIW

SEQ ID NO: 338 - protein, 12 aa, Homo sapiens
CAVSDSNYQLIW

SEQ ID NO: 339 - protein, 12 aa, Homo sapiens
CAVRDSNYQLIW

SEQ ID NO: 340 - DNA, 327 bp, Homo sapiens
  source 1..327: mol_type = genomic DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttc

SEQ ID NO: 341 - DNA, 48 bp, Homo sapiens
  source 1..48: mol_type = genomic DNA
tgtgccagcagtgaagtagccggacagggcaccggggagctgtttttt

SEQ ID NO: 342 - DNA, 39 bp, Homo sapiens
  source 1..39: mol_type = genomic DNA
tgtgccagcagcgcaggggacaccggggagctgtttttt

SEQ ID NO: 343 - DNA, 48 bp, Homo sapiens
  source 1..48: mol_type = genomic DNA
tgtgccagcagtgaagccgggacaggggacaccggggagctgtttttt

SEQ ID NO: 344 - DNA, 42 bp, Homo sapiens
  source 1..42: mol_type = genomic DNA
tgtgccagcagtgtcggagggggcaccggggagctgtttttt

SEQ ID NO: 345 - DNA, 45 bp, Homo sapiens
  source 1..45: mol_type = genomic DNA
tgtgccagcagtgacggtagcggggggaccggggagctgtttttt

SEQ ID NO: 346 - DNA, 45 bp, Homo sapiens
  source 1..45: mol_type = genomic DNA
tgtgccagcagtgacgatgcgggggggaccggggagctgtttttt

SEQ ID NO: 347 - DNA, 567 bp, Homo sapiens
  source 1..567: mol_type = genomic DNA
ggagaaggctctaggctgaccgtactggaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 348 - protein, 109 aa, Homo sapiens
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYF

SEQ ID NO: 349 - protein, 188 aa, Homo sapiens
GEGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 350 - DNA, 312 bp, Homo sapiens; identical to SEQ ID NO: 283
  source 1..312: mol_type = genomic DNA

SEQ ID NO: 351 - DNA, 36 bp, Homo sapiens; identical to SEQ ID NO: 284
  source 1..36: mol_type = genomic DNA

SEQ ID NO: 352 - DNA, 36 bp, Homo sapiens
  source 1..36: mol_type = genomic DNA
tgtgctactgcggatagcaactatcagttaatctgg

SEQ ID NO: 353 - DNA, 36 bp, Homo sapiens
  source 1..36: mol_type = genomic DNA
tgtgctgtcctggatagcaactatcagttaatctgg

SEQ ID NO: 354 - DNA, 36 bp, Homo sapiens; identical to SEQ ID NO: 290
  source 1..36: mol_type = genomic DNA

SEQ ID NO: 355 - DNA, 36 bp, Homo sapiens
  source 1..36: mol_type = genomic DNA
tgtgctgtgagtgatagcaactatcagttaatctgg

SEQ ID NO: 356 - DNA, 36 bp, Homo sapiens; identical to SEQ ID NO: 290
  source 1..36: mol_type = genomic DNA

SEQ ID NO: 357 - DNA, 456 bp, Homo sapiens; identical to SEQ ID NO: 24
  source 1..456: mol_type = genomic DNA

SEQ ID NO: 358 - protein, 104 aa, Homo sapiens; identical to SEQ ID NO: 294

SEQ ID NO: 359 - protein, 151 aa, Homo sapiens; identical to SEQ ID NO: 26

SEQ ID NO: 360 - protein, 15 aa, Homo sapiens
CASSEAGGSYNEQFF

SEQ ID NO: 361 - protein, 15 aa, Homo sapiens
CASSEAGGSYNEQFF

SEQ ID NO: 362 - protein, 15 aa, Homo sapiens
CASSESDGSYNEQFF

SEQ ID NO: 363 - protein, 12 aa, Homo sapiens
CAVMDSNYQLIW

SEQ ID NO: 364 - protein, 12 aa, Homo sapiens
CAVMDSNYQLIW

SEQ ID NO: 365 - protein, 12 aa, Homo sapiens
CAVGDSNYQLIW

SEQ ID NO: 366 - DNA, 327 bp, Homo sapiens; identical to SEQ ID NO: 340
  source 1..327: mol_type = genomic DNA

SEQ ID NO: 367 - DNA, 45 bp, Homo sapiens
  source 1..45: mol_type = genomic DNA
tgtgccagcagtgaagcggggggctcctacaatgagcagttcttc

SEQ ID NO: 368 - DNA, 45 bp, Homo sapiens; identical to SEQ ID NO: 367
  source 1..45: mol_type = genomic DNA

SEQ ID NO: 369 - DNA, 45 bp, Homo sapiens
  source 1..45: mol_type = genomic DNA
tgtgccagcagtgaaagcgacgggagctacaatgagcagttcttc

SEQ ID NO: 370 - DNA, 567 bp, Homo sapiens; identical to SEQ ID NO: 116
  source 1..567: mol_type = genomic DNA

SEQ ID NO: 371 - protein, 109 aa, Homo sapiens; identical to SEQ ID NO: 348

SEQ ID NO: 372 - protein, 188 aa, Homo sapiens; identical to SEQ ID NO: 118

SEQ ID NO: 373 - DNA, 312 bp, Homo sapiens; identical to SEQ ID NO: 283
  source 1..312: mol_type = genomic DNA

SEQ ID NO: 374 - DNA, 36 bp, Homo sapiens; identical to SEQ ID NO: 286
  source 1..36: mol_type = genomic DNA

SEQ ID NO: 375 - DNA, 36 bp, Homo sapiens; identical to SEQ ID NO: 286
  source 1..36: mol_type = genomic DNA

SEQ ID NO: 376 - DNA, 36 bp, Homo sapiens
  source 1..36: mol_type = genomic DNA
tgtgctgtgggggatagcaactatcagttaatctgg

SEQ ID NO: 377 - DNA, 456 bp, Homo sapiens; identical to SEQ ID NO: 24
  source 1..456: mol_type = genomic DNA

SEQ ID NO: 378 - protein, 104 aa, Homo sapiens; identical to SEQ ID NO: 294

SEQ ID NO: 379 - protein, 151 aa, Homo sapiens; identical to SEQ ID NO: 26

SEQ ID NO: 380 - protein, 15 aa, Homo sapiens
CASSDSRGGTDTQYF

SEQ ID NO: 381 - protein, 16 aa, Homo sapiens
CASSGYSGGGADTQYF

SEQ ID NO: 382 - protein, 15 aa, Homo sapiens
CASSDSGGSADTQYF

SEQ ID NO: 383 - protein, 15 aa, Homo sapiens
CASSGLAGGPDTQYF

SEQ ID NO: 384 - protein, 16 aa, Homo sapiens
CASSELGGGGTDTQYF

SEQ ID NO: 385 - protein, 14 aa, Homo sapiens
CASSTGGGTDTQYF

SEQ ID NO: 386 - protein, 15 aa, Homo sapiens
CASSERAGGTDTQYF

SEQ ID NO: 387 - protein, 15 aa, Homo sapiens
CASSDSAGGEDTQYF

SEQ ID NO: 388 - protein, 14 aa, Homo sapiens
CASSGGTETDTQYF

SEQ ID NO: 389 - protein, 17 aa, Homo sapiens
CASSGPTSGGPGDTQYF

SEQ ID NO: 390 - protein, 16 aa, Homo sapiens
CASSQTSGGGADTQYF

SEQ ID NO: 391 - protein, 12 aa, Homo sapiens
CAVMDSNYQLIW

SEQ ID NO: 392 - protein, 12 aa, Homo sapiens
CAVMDSNYQLIW

SEQ ID NO: 393 - protein, 12 aa, Homo sapiens
CAVRDSNYQLIW

SEQ ID NO: 394 - protein, 12 aa, Homo sapiens
CAVRDSNYQLIW

SEQ ID NO: 395 - protein, 13 aa, Homo sapiens
CAVRRDSNYQLIW

SEQ ID NO: 396 - protein, 12 aa, Homo sapiens
CAVMDSNYQLIW

SEQ ID NO: 397 - protein, 12 aa, Homo sapiens
CASSDSNYQLIW

SEQ ID NO: 398 - protein, 12 aa, Homo sapiens
CAVRDSNYQLIW

SEQ ID NO: 399 - protein, 12 aa, Homo sapiens
CAVRDSNYQLIW

SEQ ID NO: 400 - protein, 12 aa, Homo sapiens
CAVLDSNYQLIW

SEQ ID NO: 401 - protein, 12 aa, Homo sapiens
CAVRDSNYQLIW

SEQ ID NO: 402 - DNA, 327 bp, Homo sapiens; identical to SEQ ID NO: 340
  source 1..327: mol_type = genomic DNA

SEQ ID NO: 403 - DNA, 45 bp, Homo sapiens; identical to SEQ ID NO: 279
  source 1..45: mol_type = genomic DNA

SEQ ID NO: 404 - DNA, 48 bp, Homo sapiens
  source 1..48: mol_type = genomic DNA
tgtgccagcagtgggtatagcggggggggggcagatacgcagtatttt

SEQ ID NO: 405 - DNA, 45 bp, Homo sapiens; identical to SEQ ID NO: 313
  source 1..45: mol_type = genomic DNA

SEQ ID NO: 406 - DNA, 45 bp, Homo sapiens
  source 1..45: mol_type = genomic DNA
tgtgccagcagtggactagcgggggggccagatacgcagtatttt

SEQ ID NO: 407 - DNA, 48 bp, Homo sapiens
  source 1..48: mol_type = genomic DNA
tgtgccagcagtgaattggggggggggggaacagatacgcagtatttt

SEQ ID NO: 408 - DNA, 42 bp, Homo sapiens; identical to SEQ ID NO: 278
  source 1..42: mol_type = genomic DNA

SEQ ID NO: 409 - DNA, 45 bp, Homo sapiens; identical to SEQ ID NO: 275
  source 1..45: mol_type = genomic DNA

SEQ ID NO: 410 - DNA, 45 bp, Homo sapiens; identical to SEQ ID NO: 311
  source 1..45: mol_type = genomic DNA

SEQ ID NO: 411 - DNA, 42 bp, Homo sapiens
  source 1..42: mol_type = genomic DNA
tgtgccagcagtggagggaccgagacagatacgcagtatttt

SEQ ID NO: 412 - DNA, 51 bp, Homo sapiens
  source 1..51: mol_type = genomic DNA
tgtgccagcagtggcccgactagcggggggccgggagatacgcagtatttt

SEQ ID NO: 413 - DNA, 48 bp, Homo sapiens
  source 1..48: mol_type = genomic DNA
tgtgccagcagtcaaactagcggggggggtgcagatacgcagtatttt

SEQ ID NO: 414 - DNA, 567 bp, Homo sapiens; identical to SEQ ID NO: 280
  source 1..567: mol_type = genomic DNA

SEQ ID NO: 415 - protein, 109 aa, Homo sapiens; identical to SEQ ID NO: 348

SEQ ID NO: 416 - protein, 188 aa, Homo sapiens; identical to SEQ ID NO: 118

SEQ ID NO: 417 - DNA, 312 bp, Homo sapiens; identical to SEQ ID NO: 283
  source 1..312: mol_type = genomic DNA

SEQ ID NO: 418 - DNA, 36 bp, Homo sapiens; identical to SEQ ID NO: 286
  source 1..36: mol_type = genomic DNA

SEQ ID NO: 419 - DNA, 36 bp, Homo sapiens
  source 1..36: mol_type = genomic DNA
tgtgctgttatggatagcaactatcagttaatctgg

SEQ ID NO: 420 - DNA, 36 bp, Homo sapiens; identical to SEQ ID NO: 290
  source 1..36: mol_type = genomic DNA

SEQ ID NO: 421 - DNA, 36 bp, Homo sapiens; identical to SEQ ID NO: 284
  source 1..36: mol_type = genomic DNA

SEQ ID NO: 422 - DNA, 39 bp, Homo sapiens
  source 1..39: mol_type = genomic DNA
tgtgccgtgaggagggatagcaactatcagttaatctgg

SEQ ID NO: 423 - DNA, 36 bp, Homo sapiens; identical to SEQ ID NO: 291
  source 1..36: mol_type = genomic DNA

SEQ ID NO: 424 - DNA, 36 bp, Homo sapiens; identical to SEQ ID NO: 288
  source 1..36: mol_type = genomic DNA

SEQ ID NO: 425 - DNA, 36 bp, Homo sapiens; identical to SEQ ID NO: 290
  source 1..36: mol_type = genomic DNA

SEQ ID NO: 426 - DNA, 36 bp, Homo sapiens; identical to SEQ ID NO: 290
  source 1..36: mol_type = genomic DNA

SEQ ID NO: 427 - DNA, 36 bp, Homo sapiens; identical to SEQ ID NO: 353
  source 1..36: mol_type = genomic DNA

SEQ ID NO: 428 - DNA, 36 bp, Homo sapiens; identical to SEQ ID NO: 284
  source 1..36: mol_type = genomic DNA

SEQ ID NO: 429 - DNA, 456 bp, Homo sapiens; identical to SEQ ID NO: 24
  source 1..456: mol_type = genomic DNA

SEQ ID NO: 430 - protein, 104 aa, Homo sapiens; identical to SEQ ID NO: 294

SEQ ID NO: 431 - protein, 151 aa, Homo sapiens; identical to SEQ ID NO: 26

SEQ ID NO: 432 - protein, 14 aa, Homo sapiens
CASSGDGMNTEAFF

SEQ ID NO: 433 - protein, 14 aa, Homo sapiens
CASSESGENTEAFF

SEQ ID NO: 434 - protein, 13 aa, Homo sapiens
CASSPGENTEAFF

SEQ ID NO: 435 - protein, 14 aa, Homo sapiens
CASSPRGENTEAFF

SEQ ID NO: 436 - protein, 12 aa, Homo sapiens
CAPLDSNYQLIW

SEQ ID NO: 437 - protein, 12 aa, Homo sapiens
CAAQDSNYQLIW

SEQ ID NO: 438 - protein, 12 aa, Homo sapiens
CAAMDSNYQLIW

SEQ ID NO: 439 - protein, 12 aa, Homo sapiens
CAALDSNYQLIW

SEQ ID NO: 440 - DNA, 42 bp, Homo sapiens
  source 1..42: mol_type = genomic DNA
tgcgccagcagtggggacggaatgaacactgaagctttcttt

SEQ ID NO: 441 - DNA, 42 bp, Homo sapiens
  source 1..42: mol_type = genomic DNA
tgtgccagcagtgaatccggagagaacactgaagctttcttt

SEQ ID NO: 442 - DNA, 39 bp, Homo sapiens
  source 1..39: mol_type = genomic DNA
tgtgccagcagtcctggggaaaatactgaagctttcttt

SEQ ID NO: 443 - DNA, 42 bp, Homo sapiens
  source 1..42: mol_type = genomic DNA
tgtgccagcagccccaggggggagaacactgaagctttcttt

SEQ ID NO: 444 - DNA, 39 bp, Homo sapiens
  source 1..39: mol_type = genomic DNA
tgtgctgtcctgatggatagcaactatcagttaatctgg

SEQ ID NO: 445 - DNA, 39 bp, Homo sapiens
  source 1..39: mol_type = genomic DNA
tgtgctgtactcatggatagcaactatcagttaatctgg

SEQ ID NO: 446 - DNA, 36 bp, Homo sapiens
  source 1..36: mol_type = genomic DNA
tgtgctccattggatagcaactatcagttaatctgg

SEQ ID NO: 447 - DNA, 36 bp, Homo sapiens
  source 1..36: mol_type = genomic DNA
tgtgctgcccaggatagcaactatcagttaatctgg

SEQ ID NO: 448 - DNA, 36 bp, Homo sapiens
  source 1..36: mol_type = genomic DNA
tgtgctgccatggatagcaactatcagttaatctgg

SEQ ID NO: 449 - DNA, 36 bp, Homo sapiens
  source 1..36: mol_type = genomic DNA
tgtgctgctctggatagcaactatcagttaatctgg

SEQ ID NO: 450 - protein, 15 aa, Homo sapiens
CASSISSGSYNEQFF

SEQ ID NO: 451 - protein, 15 aa, Homo sapiens
CASSISSGSYNEQFF

SEQ ID NO: 452 - protein, 15 aa, Homo sapiens
CASSITSGSYDEQFF

SEQ ID NO: 453 - protein, 15 aa, Homo sapiens
CASSITSGSYNEQFF

SEQ ID NO: 454 - protein, 15 aa, Homo sapiens
CASTMTSGAYNEQFF

SEQ ID NO: 455 - protein, 15 aa, Homo sapiens
CVVTPGREYGNKLVF

SEQ ID NO: 456 - protein, 15 aa, Homo sapiens
CVVSSGREYGNKLVF

SEQ ID NO: 457 - protein, 15 aa, Homo sapiens
CVVSTGREYGNKLVF

SEQ ID NO: 458 - protein, 15 aa, Homo sapiens
CVVSAGREYGNKLVF

SEQ ID NO: 459 - protein, 15 aa, Homo sapiens
CVVSAGREYGNKLVF

SEQ ID NO: 460 - DNA, 45 bp, Homo sapiens
  source 1..45: mol_type = genomic DNA
tgtgccagtagtatttctagcggatcctacaatgagcagttcttc

SEQ ID NO: 461 - DNA, 45 bp, Homo sapiens
  source 1..45: mol_type = genomic DNA
tgtgccagtagtataagtagcgggagctacaatgagcagttcttc

SEQ ID NO: 462 - DNA, 45 bp, Homo sapiens
  source 1..45: mol_type = genomic DNA
tgtgccagtagtataactagcgggagttacgatgagcagttcttc

SEQ ID NO: 463 - DNA, 45 bp, Homo sapiens
  source 1..45: mol_type = genomic DNA
tgtgccagtagtataactagcggttcctacaatgagcagttcttc

SEQ ID NO: 464 - DNA, 45 bp, Homo sapiens
  source 1..45: mol_type = genomic DNA
tgtgccagtaccatgactagcggggcctacaatgagcagttcttc

SEQ ID NO: 465 - DNA, 45 bp, Homo sapiens
  source 1..45: mol_type = genomic DNA
tgtgtggtgacccccggtagggaatatggaaacaaactggtcttt

SEQ ID NO: 466 - DNA, 45 bp, Homo sapiens
  source 1..45: mol_type = genomic DNA
tgtgtggtgagctccggcagggaatatggaaacaaactggtcttt

SEQ ID NO: 467 - DNA, 45 bp, Homo sapiens
  source 1..45: mol_type = genomic DNA
tgtgtggtgagcacggggagggaatatggaaacaaactggtcttt

SEQ ID NO: 468 - DNA, 45 bp, Homo sapiens
  source 1..45: mol_type = genomic DNA
tgtgtggtgagcgcgggtcgggaatatggaaacaaactggtcttt

SEQ ID NO: 469 - DNA, 45 bp, Homo sapiens
  source 1..45: mol_type = genomic DNA
tgtgtggtgagcgcgggtagggaatatggaaacaaactggtcttt

SEQ ID NO: 470 - protein, 15 aa, Homo sapiens
CASSITSGDYNEQFF

SEQ ID NO: 471 - protein, 15 aa, Homo sapiens
CASSMTSGSYNEQFF

SEQ ID NO: 472 - protein, 18 aa, Homo sapiens
CAVRRGRDGGTSYGKLTF

SEQ ID NO: 473 - protein, 19 aa, Homo sapiens
CAVRASRDAGGTSYGKLTF

SEQ ID NO: 474 - DNA, 45 bp, Homo sapiens
  source 1..45: mol_type = genomic DNA
tgtgccagtagtattacttcgggggattacaatgagcagttcttc

SEQ ID NO: 475 - DNA, 45 bp, Homo sapiens
  source 1..45: mol_type = genomic DNA
tgtgccagtagtatgactagcgggtcctacaatgagcagttcttc

SEQ ID NO: 476 - DNA, 54 bp, Homo sapiens
  source 1..54: mol_type = genomic DNA
tgtgccgtgaggaggggtcgagatggtggtactagctatggaaagctgacattt

SEQ ID NO: 477 - DNA, 57 bp, Homo sapiens
  source 1..57: mol_type = genomic DNA
tgtgccgtgagggcttcgagggatgctggtggtactagctatggaaagctgacattt

SEQ ID NO: 478 - protein, 13 aa, Homo sapiens
CSARDQTGNTIYF

SEQ ID NO: 479 - DNA, 39 bp, Homo sapiens
  source 1..39: mol_type = genomic DNA
tgcagtgctagagatcagaccggaaacaccatatatttt

SEQ ID NO: 480 - DNA, 33 bp, Homo sapiens
  source 1..33: mol_type = genomic DNA
tgtgcagaggacgaaaatgccagactcatgttt

SEQ ID NO: 481 - protein, 15 aa, Homo sapiens
CASSDSSGGQETQYF

SEQ ID NO: 482 - protein, 15 aa, Homo sapiens
CASSDSSGGTDTQYF

SEQ ID NO: 483 - protein, 15 aa, Homo sapiens
CASSDSSGGTDTQYF

SEQ ID NO: 484 - protein, 12 aa, Homo sapiens
CAVMDSNYQLIW

SEQ ID NO: 485 - protein, 12 aa, Homo sapiens
CAVRDSNYQLIW

SEQ ID NO: 486 - protein, 12 aa, Homo sapiens
CAVRDSNYQLIW

SEQ ID NO: 487 - DNA, 45 bp, Homo sapiens
  source 1..45: mol_type = genomic DNA
tgtgccagcagtgactctagcggggggcaagagacccagtacttc

SEQ ID NO: 488 - DNA, 45 bp, Homo sapiens
  source 1..45: mol_type = genomic DNA
tgtgccagcagtgactctagcgggggcacagatacgcagtatttt

SEQ ID NO: 489 - DNA, 45 bp, Homo sapiens
  source 1..45: mol_type = genomic DNA
tgtgccagcagtgactcctcagggggcacagatacgcagtatttt

SEQ ID NO: 490 - DNA, 36 bp, Homo sapiens; identical to SEQ ID NO: 286
  source 1..36: mol_type = genomic DNA

SEQ ID NO: 491 - DNA, 36 bp, Homo sapiens; identical to SEQ ID NO: 290
  source 1..36: mol_type = genomic DNA

SEQ ID NO: 492 - DNA, 36 bp, Homo sapiens; identical to SEQ ID NO: 290
  source 1..36: mol_type = genomic DNA

SEQ ID NO: 493 - protein, 14 aa, Homo sapiens
CASSDSADTDTQYF

SEQ ID NO: 494 - protein, 15 aa, Homo sapiens
CASSDGQGGTDTQYF

SEQ ID NO: 495 - protein, 13 aa, Homo sapiens
CASSDQVTDTQYF

SEQ ID NO: 496 - protein, 12 aa, Homo sapiens
CAVQDSNYQLIW

SEQ ID NO: 497 - protein, 12 aa, Homo sapiens
CAVKDSNYQLIW

SEQ ID NO: 498 - protein, 12 aa, Homo sapiens
CAVRDSNYQLIW

SEQ ID NO: 499 - DNA, 42 bp, Homo sapiens
  source 1..42: mol_type = genomic DNA
tgtgccagcagtgactcggctgacacagatacgcagtatttt

SEQ ID NO: 500 - DNA, 45 bp, Homo sapiens
  source 1..45: mol_type = genomic DNA
tgtgccagcagtgacggccagggtggaacagatacgcagtatttt

SEQ ID NO: 501 - DNA, 39 bp, Homo sapiens
  source 1..39: mol_type = genomic DNA
tgtgccagcagtgaccaggttacagatacgcagtatttt

SEQ ID NO: 502 - DNA, 36 bp, Homo sapiens
  source 1..36: mol_type = genomic DNA
tgtgctgtgcaggatagcaactatcagttaatctgg

SEQ ID NO: 503 - DNA, 36 bp, Homo sapiens; identical to SEQ ID NO: 324
  source 1..36: mol_type = genomic DNA

SEQ ID NO: 504 - DNA, 36 bp, Homo sapiens; identical to SEQ ID NO: 290
  source 1..36: mol_type = genomic DNA

SEQ ID NO: 505 - protein, 15 aa, Homo sapiens
CASSELAGGPDTQYF

SEQ ID NO: 506 - protein, 15 aa, Homo sapiens
CASSELAGGPETQYF

SEQ ID NO: 507 - protein, 12 aa, Homo sapiens
CAVRDSNYQLIW

SEQ ID NO: 508 - protein, 12 aa, Homo sapiens
CAVRDSNYQLIW

SEQ ID NO: 509 - DNA, 45 bp, Homo sapiens
  source 1..45: mol_type = genomic DNA
tgtgccagcagtgaactagcgggggggccagatacgcagtatttt

SEQ ID NO: 510 - DNA, 45 bp, Homo sapiens
  source 1..45: mol_type = genomic DNA
tgtgccagcagtgaattagcgggggggccagagacccagtacttc

SEQ ID NO: 511 - DNA, 36 bp, Homo sapiens; identical to SEQ ID NO: 290
  source 1..36: mol_type = genomic DNA

SEQ ID NO: 512 - DNA, 36 bp, Homo sapiens; identical to SEQ ID NO: 290
  source 1..36: mol_type = genomic DNA

SEQ ID NO: 513 - protein, 11 aa, Homo sapiens
CAEDENARLMF